SEQUENCE LISTING (40 sequences)

SEQ ID NO: 1 - protein, 11 aa, synthetic construct
  VARIANT 1: note = L- or D-amino acid
  VARIANT 2: note = Any amino acid
  MOD_RES 4: note = 4Hyp
  MOD_RES 5: note = 3-chloro-L-tyrosine
  VARIANT 6: note = Any amino acid
  VARIANT 11: note = Any amino acid
  VARIANT 9: note = Any amino acid
YXCPYXLCXIX

SEQ ID NO: 2 - protein, 11 aa, synthetic construct
  MOD_RES 1..2: note = D-amino acid
  MOD_RES 4: note = 4Hyp
  MOD_RES 5: note = 3-chloro-L-tyrosine
YECPYGLCHIQ

SEQ ID NO: 3 - protein, 11 aa, synthetic construct
  MOD_RES 1..2: note = D-amino acid
  MOD_RES 4: note = 4Hyp
  MOD_RES 5: note = 3-chloro-L-tyrosine
  MOD_RES 11: note = D-amino acid
YECPYGLCHIL

SEQ ID NO: 4 - protein, 11 aa, synthetic construct
  MOD_RES 2: note = D-amino acid
  MOD_RES 4: note = 4Hyp
  MOD_RES 5: note = 3-chloro-L-tyrosine
YECPYGLCHIQ

SEQ ID NO: 5 - protein, 11 aa, synthetic construct
  MOD_RES 2: note = D-amino acid
  MOD_RES 4: note = 4Hyp
  MOD_RES 5: note = 3-chloro-L-tyrosine
  MOD_RES 11: note = D-amino acid
YECPYGLCHIQ

SEQ ID NO: 6 - protein, 11 aa, synthetic construct
  MOD_RES 1..2: note = D-amino acid
  MOD_RES 4: note = 4Hyp
  MOD_RES 5: note = 3-chloro-L-tyrosine
YECPYGLCYIQ

SEQ ID NO: 7 - protein, 11 aa, synthetic construct
  MOD_RES 2: note = D-amino acid
  MOD_RES 4: note = 4Hyp
  MOD_RES 5: note = 3-chloro-L-tyrosine
YECPYGLCYIQ

SEQ ID NO: 8 - protein, 11 aa, synthetic construct
  MOD_RES 2: note = D-amino acid
  MOD_RES 4: note = 4Hyp
  MOD_RES 5: note = 3-chloro-L-tyrosine
  MOD_RES 11: note = D-amino acid
YECPYGLCHIL

SEQ ID NO: 9 - protein, 11 aa, synthetic construct
  MOD_RES 1..2: note = D-amino acid
  MOD_RES 4: note = 4Hyp
  MOD_RES 5: note = 3-chloro-L-tyrosine
  MOD_RES 11: note = D-amino acid
YECPYGLCYIL

SEQ ID NO: 10 - protein, 11 aa, synthetic construct
  MOD_RES 2: note = D-amino acid
  MOD_RES 4: note = 4Hyp
  MOD_RES 5: note = 3-chloro-L-tyrosine
  MOD_RES 11: note = D-amino acid
YECPYGLCYIL

SEQ ID NO: 11 - protein, 11 aa, synthetic construct
  MOD_RES 1..2: note = D-amino acid
  MOD_RES 4: note = 4Hyp
  MOD_RES 5: note = 3-chloro-L-tyrosine
  MOD_RES 11: note = D-amino acid
YECPYGLCYIQ

SEQ ID NO: 12 - protein, 11 aa, synthetic construct
  MOD_RES 2: note = D-amino acid
  MOD_RES 4: note = 4Hyp
  MOD_RES 5: note = 3-chloro-L-tyrosine
YECPYDLCYIQ

SEQ ID NO: 13 - protein, 11 aa, synthetic construct
  MOD_RES 2: note = D-amino acid
  MOD_RES 4: note = 4Hyp
  MOD_RES 5: note = 3-chloro-L-tyrosine
  MOD_RES 11: note = D-amino acid
YECPYGLCYIQ

SEQ ID NO: 14 - protein, 11 aa, synthetic construct
  MOD_RES 2: note = D-amino acid
  MOD_RES 4: note = 4Hyp
  MOD_RES 5: note = 3-chloro-L-tyrosine
  MOD_RES 6: note = D-amino acid
YECPYDLCYIQ

SEQ ID NO: 15 - protein, 11 aa, synthetic construct
  MOD_RES 2: note = D-amino acid
  MOD_RES 4: note = 4Hyp
  MOD_RES 5: note = 3-chloro-L-tyrosine
YHCPYGLCYIQ

SEQ ID NO: 16 - protein, 11 aa, synthetic construct
  VARIANT 1: note = L- or D-amino acid
  VARIANT 2: note = Any amino acid
  MOD_RES 4: note = 4Hyp
  MOD_RES 5: note = 3-chloro-L-tyrosine
  VARIANT 6: note = Any amino acid
  VARIANT 9: note = Any amino acid
  VARIANT 11: note = Any amino acid
YXCPYXLCXIX

SEQ ID NO: 17 - protein, 11 aa, synthetic construct
  VARIANT 1: note = L- or D-amino acid
  VARIANT 2: note = Any amino acid
  MOD_RES 4: note = 4Hyp
  MOD_RES 5: note = 3-chloro-L-tyrosine
  VARIANT 6: note = Any amino acid
  VARIANT 9: note = Any amino acid
  VARIANT 11: note = Any amino acid
YXCPYXLCXIX

SEQ ID NO: 18 - protein, 11 aa, synthetic construct
  MOD_RES 2: note = D-amino acid
  MOD_RES 4: note = 4Hyp
  MOD_RES 5: note = 3-chloro-L-tyrosine
YECPYGLCYIQ

SEQ ID NO: 19 - protein, 11 aa, synthetic construct
  MOD_RES 1..2: note = D-amino acid
  MOD_RES 4: note = 4Hyp
  MOD_RES 5: note = 3-chloro-L-tyrosine
YECPYGLCYIQ

SEQ ID NO: 20 - protein, 11 aa, synthetic construct
  MOD_RES 2: note = D-amino acid
  MOD_RES 4: note = 4Hyp
  MOD_RES 5: note = 3-chloro-L-tyrosine
YECPYGLCHIQ

SEQ ID NO: 21 - protein, 11 aa, synthetic construct
  MOD_RES 1..2: note = D-amino acid
  MOD_RES 4: note = 4Hyp
  MOD_RES 5: note = 3-chloro-L-tyrosine
YECPYGLCHIQ

SEQ ID NO: 22 - protein, 11 aa, synthetic construct
  MOD_RES 2: note = D-amino acid
  MOD_RES 4: note = 4Hyp
  MOD_RES 5: note = 3-chloro-L-tyrosine
  MOD_RES 11: note = D-amino acid
YECPYGLCHIQ

SEQ ID NO: 23 - protein, 11 aa, synthetic construct
  MOD_RES 1..2: note = D-amino acid
  MOD_RES 4: note = 4Hyp
  MOD_RES 5: note = 3-chloro-L-tyrosine
YECPYGLCHII

SEQ ID NO: 24 - protein, 11 aa, synthetic construct
  MOD_RES 1..3: note = D-amino acid
  MOD_RES 4: note = 4Hyp
  MOD_RES 5: note = 3-chloro-L-tyrosine
  MOD_RES 11: note = D-amino acid
YECPYGLCHIQ

SEQ ID NO: 25 - protein, 11 aa, synthetic construct
  MOD_RES 2: note = D-amino acid
  MOD_RES 4: note = 4Hyp
  MOD_RES 5: note = 3-chloro-L-tyrosine
  MOD_RES 11: note = D-amino acid
YECPYGLCHIQ

SEQ ID NO: 26 - protein, 11 aa, synthetic construct
  MOD_RES 2: note = D-amino acid
  MOD_RES 4: note = 4Hyp
  MOD_RES 5: note = 3-chloro-L-tyrosine
  MOD_RES 11: note = D-amino acid
YECPYGLCHIQ

SEQ ID NO: 27 - protein, 11 aa, synthetic construct
  MOD_RES 1..2: note = D-amino acid
  MOD_RES 4: note = 4Hyp
  MOD_RES 5: note = 3-chloro-L-tyrosine
YECPYGLCHIQ

SEQ ID NO: 28 - protein, 11 aa, synthetic construct
  MOD_RES 1..2: note = D-amino acid
  MOD_RES 4: note = 4Hyp
  MOD_RES 5: note = 3-chloro-L-tyrosine
  MOD_RES 11: note = D-amino acid
YECPYGLCHIL

SEQ ID NO: 29 - protein, 11 aa, synthetic construct
  MOD_RES 2: note = D-amino acid
  MOD_RES 4: note = 4Hyp
  MOD_RES 5: note = 3-chloro-L-tyrosine
YECPYGLCHIQ

SEQ ID NO: 30 - protein, 11 aa, synthetic construct
  MOD_RES 2: note = D-amino acid
  MOD_RES 4: note = 4Hyp
  MOD_RES 5: note = 3-chloro-L-tyrosine
  MOD_RES 11: note = D-amino acid
YECPYGLCHIQ

SEQ ID NO: 31 - protein, 11 aa, synthetic construct
  MOD_RES 1..2: note = D-amino acid
  MOD_RES 4: note = 4Hyp
  MOD_RES 5: note = 3-chloro-L-tyrosine
YECPYGLCYIQ

SEQ ID NO: 32 - protein, 11 aa, synthetic construct
  MOD_RES 2: note = D-amino acid
  MOD_RES 4: note = 4Hyp
  MOD_RES 5: note = 3-chloro-L-tyrosine
YECPYGLCYIQ

SEQ ID NO: 33 - protein, 11 aa, synthetic construct
  MOD_RES 2: note = D-amino acid
  MOD_RES 4: note = 4Hyp
  MOD_RES 5: note = 3-chloro-L-tyrosine
  MOD_RES 11: note = D-amino acid
YECPYGLCHIL

SEQ ID NO: 34 - protein, 11 aa, synthetic construct
  MOD_RES 1..2: note = D-amino acid
  MOD_RES 4: note = 4Hyp
  MOD_RES 5: note = 3-chloro-L-tyrosine
  MOD_RES 11: note = D-amino acid
YECPYGLCYIL

SEQ ID NO: 35 - protein, 11 aa, synthetic construct
  MOD_RES 2: note = D-amino acid
  MOD_RES 4: note = 4Hyp
  MOD_RES 5: note = 3-chloro-L-tyrosine
  MOD_RES 11: note = D-amino acid
YECPYGLCYIL

SEQ ID NO: 36 - protein, 11 aa, synthetic construct
  MOD_RES 1..2: note = D-amino acid
  MOD_RES 4: note = 4Hyp
  MOD_RES 5: note = 3-chloro-L-tyrosine
  MOD_RES 11: note = D-amino acid
YECPYGLCYIQ

SEQ ID NO: 37 - protein, 11 aa, synthetic construct
  MOD_RES 2: note = D-amino acid
  MOD_RES 4: note = 4Hyp
  MOD_RES 5: note = 3-chloro-L-tyrosine
YECPYDLCYIQ

SEQ ID NO: 38 - protein, 11 aa, synthetic construct
  MOD_RES 2: note = D-amino acid
  MOD_RES 4: note = 4Hyp
  MOD_RES 5: note = 3-chloro-L-tyrosine
  MOD_RES 11: note = D-amino acid
YECPYGLCYIQ

SEQ ID NO: 39 - protein, 11 aa, synthetic construct
  MOD_RES 2: note = D-amino acid
  MOD_RES 4: note = 4Hyp
  MOD_RES 5: note = 3-chloro-L-tyrosine
  MOD_RES 6: note = D-amino acid
YECPYDLCYIQ

SEQ ID NO: 40 - protein, 11 aa, synthetic construct
  MOD_RES 2: note = D-amino acid
  MOD_RES 4: note = 4Hyp
  MOD_RES 5: note = 3-chloro-L-tyrosine
YHCPYGLCYIQ

(19) United States
(12) Patent Application Publication
(10) Pub. No.: US 2026/0325688 A1
(43) Pub. Date: Oct. 22, 2026

(54) FIBRIN-BINDING COMPOUNDS FOR IMAGING AND TREATMENT

(71) Applicant: LUMINA PHARMACEUTICALS INC., Boston, MA (US)

(72) Inventors: Peter Caravan, Cambridge, MA (US); Thomas J. McMurry, Winchester, MA (US); Richard J. Looby, Reading, MA (US)

(73) Assignee: LUMINA PHARMACEUTICALS INC., Boston, MA (US)

(21) Appl. No.: 19/718,839

(22) Filed: Jun. 24, 2026

Related U.S. Application Data
(63) Continuation of application No. 17/767,956, filed on Oct. 23, 2020.
(60) Provisional application No. 62/924,997, filed on Oct. 23, 2019.

(51) Int. Cl. A61K 51/08; A61K 45/06; A61K 51/04

(52) U.S. Cl. CPC A61K 51/088; A61K 45/06; A61K 51/0482

(57) ABSTRACT

This disclosure relates to fibrin-binding compounds comprising a radioactive moiety for diagnostic imaging and treatment of various diseases and conditions associated with the presence of fibrin.

### CROSS-REFERENCE TO RELATED APPLICATIONS

This application is a continuation of U.S. patent application Ser. No. 17/767,956, filed Apr. 11, 2022, which is a national stage application filed under 35 U.S.C § 371 of international Patent Application No. PCT/US2020/057198, filed Oct. 23, 2020, which claims the benefit of U.S. Provisional Application No. 62/924,997, filed Oct. 23, 2019. The entirety of each of these applications is hereby incorporated by reference herein for all purposes.

### FEDERALLY SPONSORED RESEARCH OR DEVELOPMENT

This invention was made with Government support under Contract No. HHSN268201400044C awarded by the National Institutes of Health. The Government has certain rights in the invention.

### INCORPORATION BY REFERENCE

The Sequence Listing XML file named “25111-008WO1US2_ST26_1” created on Jun. 24, 2026, and having a size of 91,841 bytes, is hereby incorporated by reference.

### TECHNICAL FIELD

This disclosure relates to fibrin-binding compounds comprising a radioactive moiety for diagnostic imaging and treatment of various diseases and conditions associated with the presence of fibrin.

### BACKGROUND

Fibrin is a fibrillary, non-globular protein derived from the soluble plasma protein fibrinogen and is a major component of blood clots (thrombi). Polymerization of fibrinogen as facilitated by the protease thrombin forms fibrin, which, together with platelets, leads to the formation of thrombi over a wound site, thus stopping further bleeding. Fibrin is present in all thrombi, regardless of thrombus age or bodily location, and is useful in the diagnosis and treatment of diseases and conditions where the presence of fibrin is implicated. Diagnostic imaging techniques such as magnetic resonance imaging (MRI), X-ray, and nuclear radiopharmaceutical imaging including positron-emission tomography (PET) and single-photon emission computerized tomography (SPECT), are often used in the diagnosis of cardiovascular events. One approach relies on thrombus visualization via specific molecular targets, including fibrin.

Fibrin is also known to play an important role in the pathophysiology of malignancy (Costantini and Zacharski, 1992, Cancer and Metastasis Rev., 11, 283). In cancer, tumor invasion and metastasis can result in the erosion of adjacent vascular tissues leading to hemorrhage, subsequent formation of thrombi within the tumor, and replacement by collagen in a similar fashion as the normal wound healing process (see, for example, Falanga et al., 2013, J Thromb Haemost, 11, 223-233; Obonai, et al., 2016, Sci. Rep., 6, 23613). Unlike the normal wound healing process where the thrombi only form at the onset of the wound and will eventually disappear as a result of plasmin digestion or replacement with collagen, fibrin clots in cancer persist for as long as the cancer cells survive in the body. Deposition of insoluble fibrin in various tumor tissues and thrombi is correlated to the aggressiveness and progression of the tumor. Thus, there is a need for fibrin-targeting agents that can be used for the diagnosis and treatment of various cancers.

Fibrin deposits are also known to be associated with neurodegenerative diseases associated with systemic inflammation (neuroinflammation), such as Alzheimer's disease (AD), multiple sclerosis, and traumatic brain injury (TBI). Fibrin deposits have been associated with memory reduction in neuroinflammatory diseases, including AD and TBI. (Sulimai and Lominadze, 2020, Mol. Neurobiol., 57, 4692-4703). Thus, there is also a need for fibrin-targeting agents that can be used for the diagnosis and treatment of neuroinflammation.

Provided herein are fibrin-specific binding compounds, as well as methods for imaging fibrin. Also provided are methods of treating various diseases and conditions associated with the presence of fibrin, including cardiovascular diseases, cerebrovascular diseases and cancer.

### SUMMARY

Provided in the present disclosure is a compound of Formula IV:

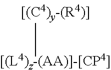

or a pharmaceutically acceptable salt thereof,
   wherein R⁴ is a radioisotope;
   C⁴ is a chelating moiety selected from the group consisting of:

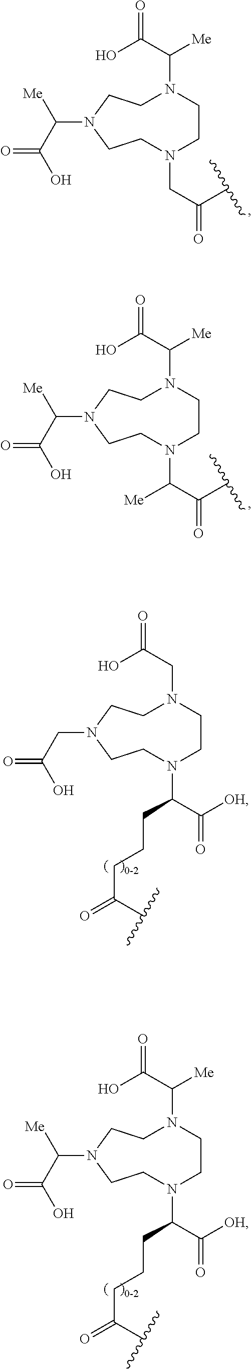

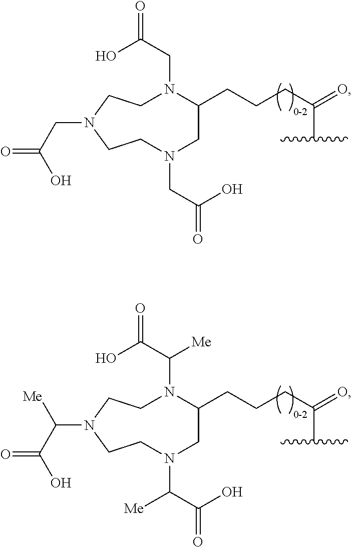

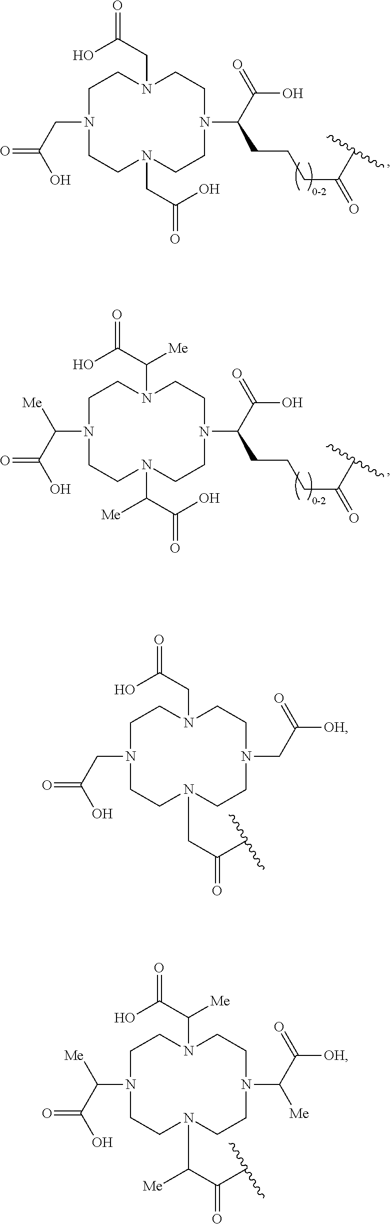

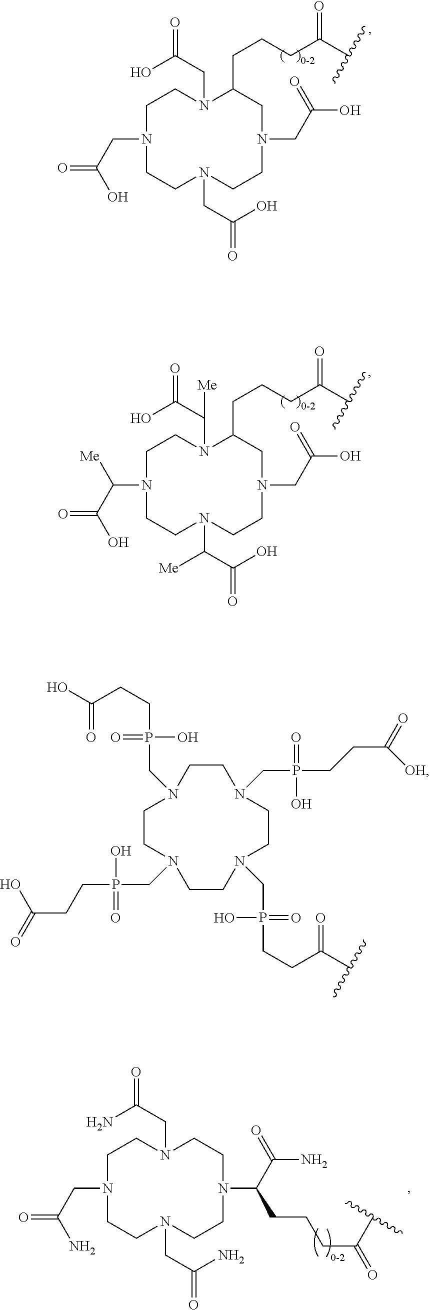

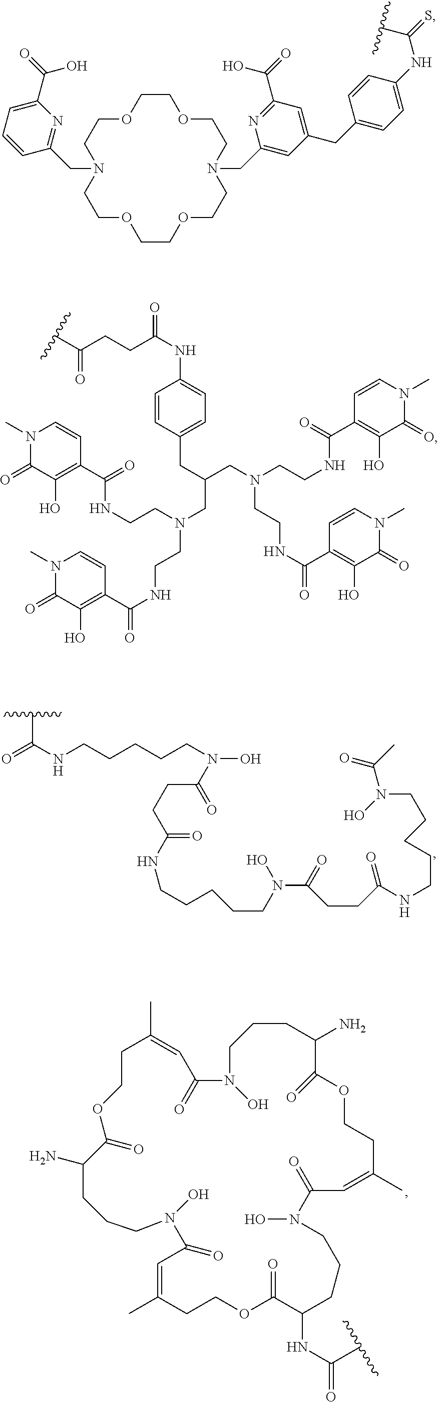 [SMILES: *C(=S)Nc1ccc(Cc2cc(CN3CCOCCOCCN(Cc4cccc(C(=O)O)n4)CCOCCOCC3)nc(C(=O)O)c2)cc1 ; *C(=O)CCC(=O)Nc1ccc(CC(CN(CCNC(=O)c2ccn(C)c(=O)c2O)CCNC(=O)c2ccn(C)c(=O)c2O)CN(CCNC(=O)c2ccn(C)c(=O)c2O)CCNC(=O)c2ccn(C)c(=O)c2O)cc1 ; *C(=O)NCCCCCN(O)C(=O)CCC(=O)NCCCCCN(O)C(=O)CCC(=O)NCCCCCN(O)C(C)=O ; *C(=O)NC1CCCN(O)C(=O)/C=C(/C)CCOC(=O)C(N)CCCN(O)C(=O)/C=C(/C)CCOC(=O)C(N)CCCN(O)C(=O)/C=C(/C)CCOC1=O]

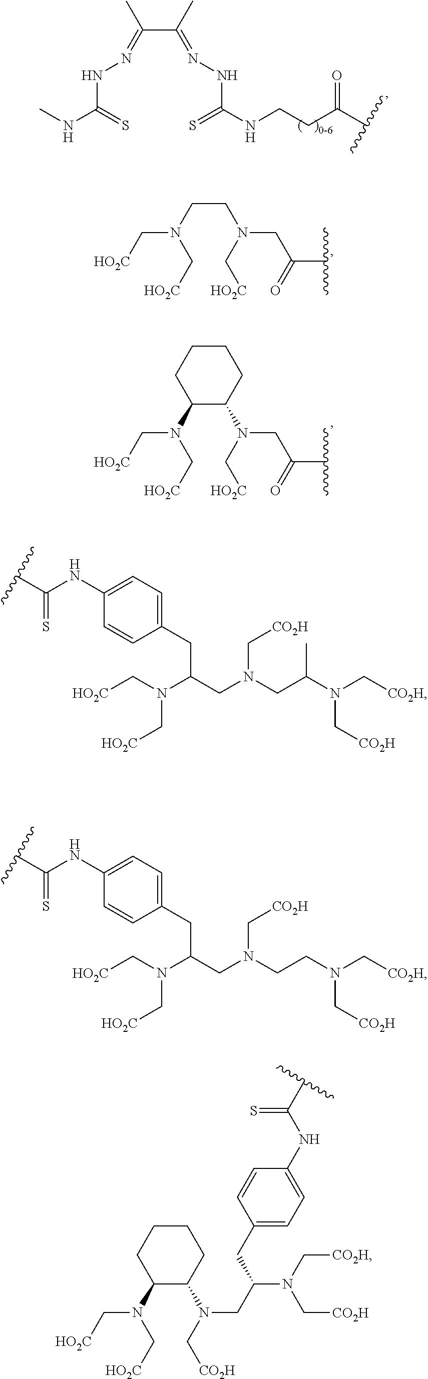

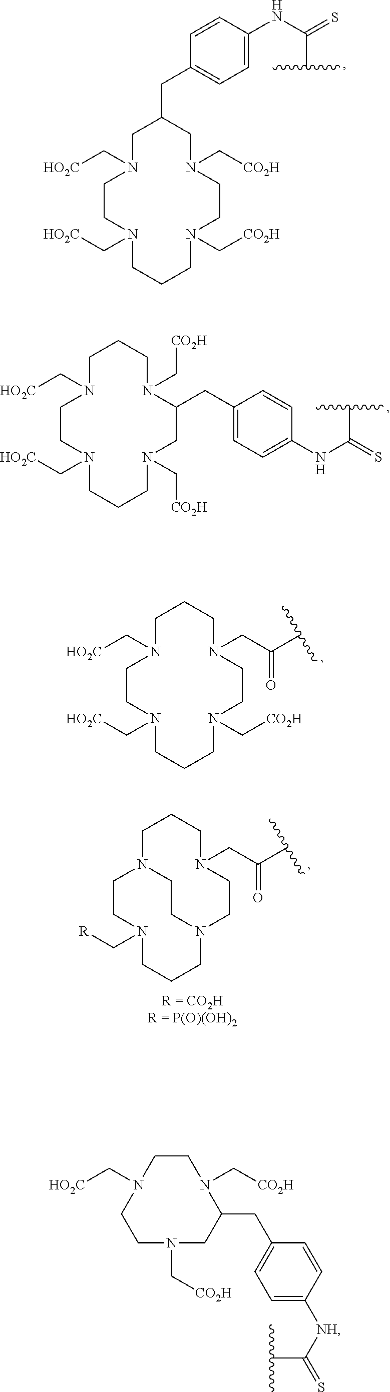

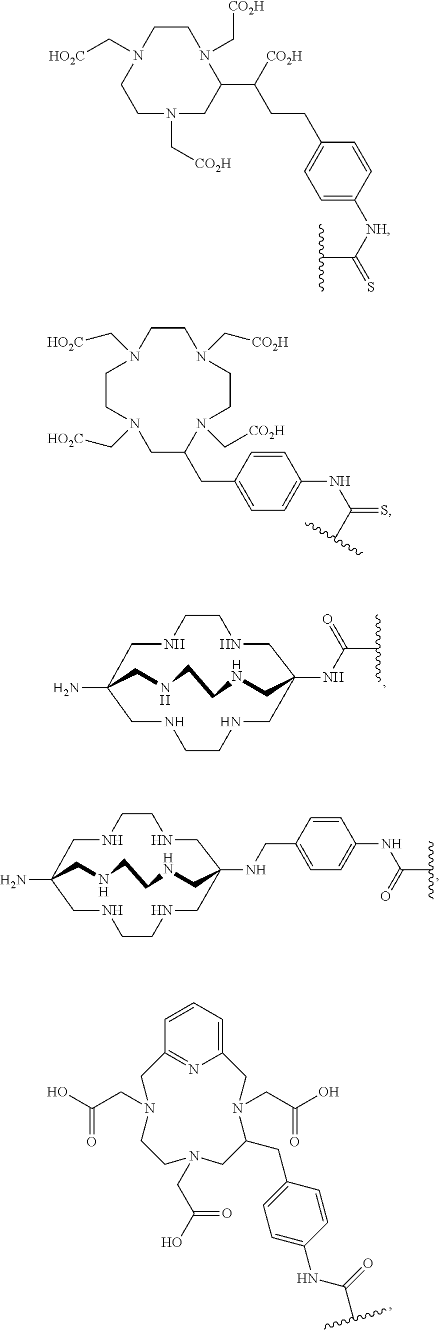

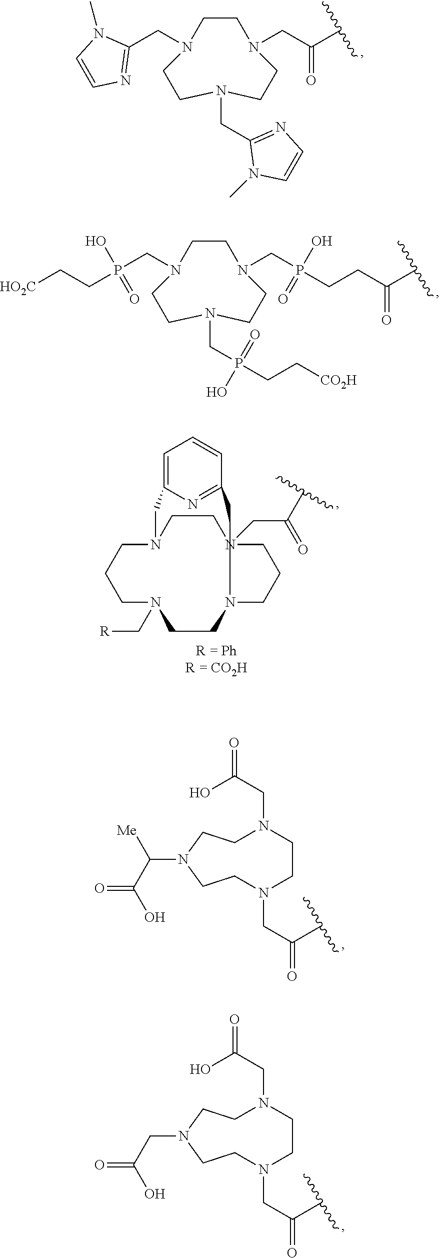

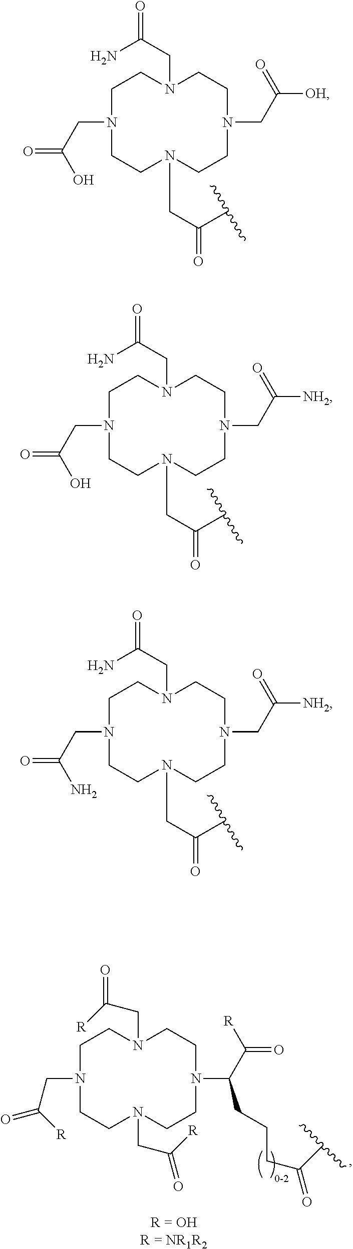

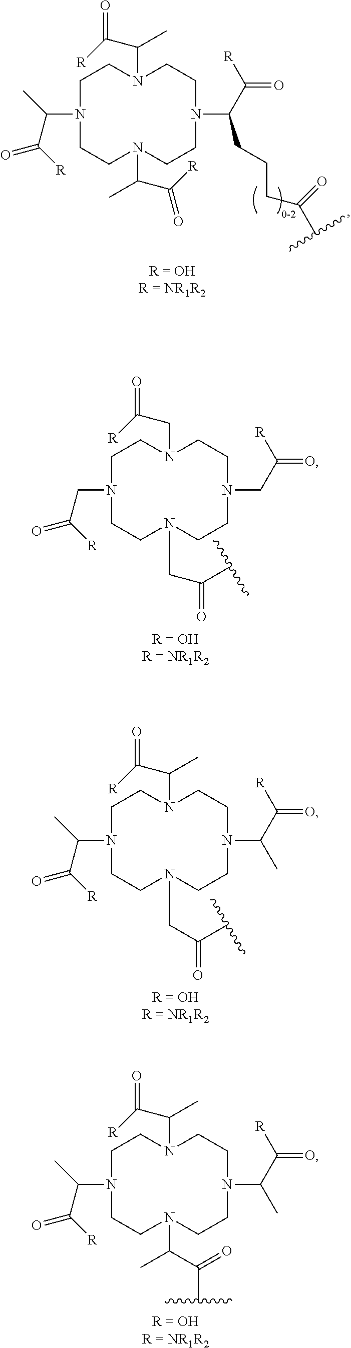

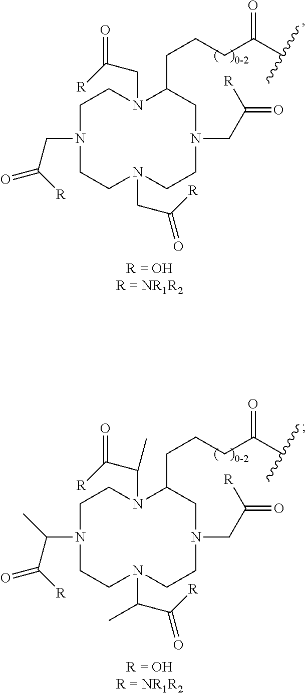

CP⁴ is a fibrin-binding peptide;
AA is the N-terminal amino acid of the fibrin-binding peptide;
L⁴ is a linker;
y is an integer selected from 0 and 1; and
z is an integer selected from 0 and 1.

In some embodiments of the compound of Formula IV, R⁴ is a radioisotope selected from a therapeutic radioisotope and a radioisotope capable of detection using a nuclear imaging technique. In some embodiments, the radioisotope capable of detection using a nuclear imaging technique is a positron emitting isotope or a radioisotope suitable for single-photon emission computerized tomography (SPECT) imaging. In some embodiments, the positron emitting isotope is selected from the group consisting of fluorine-18, aluminum fluoride (Al¹¹8F), scandium-43, scandium-44, manganese-51, manganese-52, copper-60, copper-61, copper-62, copper-64, gallium-68, yttrium-86, zirconium-89, iodine-124, terbium-149, and terbium-152. In some embodiments, the positron emitting isotope is selected from the group consisting of fluorine-18, copper-64, and gallium-68. In some embodiments, the positron emitting isotope is fluorine-18. In some embodiments, the positron emitting isotope is copper-64. In some embodiments, the positron emitting isotope is gallium-68. In some embodiments, the radioisotope suitable for SPECT imaging is selected from the group consisting of gallium-67, technetium-99m, indium-111, iodine-123, terbium-155, and lead-203.

In some embodiments, the radioisotope is a therapeutic radioisotope (e.g., a beta emitter or an alpha emitter). In some embodiments, the therapeutic radioisotope is selected from the group consisting of scandium-47, copper-67, yttrium-90, iodine-125, iodine-131, samarium-153, terbium-161, holmium-166, lutetium-177, rhenium-188, astatine-211, lead-212, bismuth-213, radium-223, actinium-225, and thorium-227. In some embodiments, the therapeutic isotope is selected from the group consisting of yttrium-90, lutetium-177, and actinium-225. In some embodiments, the therapeutic isotope is yttrium-90. In some embodiments, the therapeutic isotope is lutetium-177. In some embodiments, the therapeutic isotope is actinium-225.

In some embodiments of the compound of Formula IV, AA-CP⁴ is a fibrin-binding peptide comprising a sequence having at least 80% sequence identity to the polypeptide of SEQ ID NO:1:

(SEQ ID NO: 1)
-y*-X¹-C-Hyp-Y(3-Cl)-X²-L-C-X³-I-X⁴-

wherein each of X¹, X², X³, and X⁴ is independently any amino acid; and y* is L-tyrosine or D-tyrosine.

In some embodiments of the compound of Formula IV, AA-CP⁴ is a fibrin-binding peptide comprising a polypeptide having at least 80% sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments of the compound of Formula IV, AA-CP⁴ is a fibrin-binding peptide having at least 80% sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments of the compound of Formula IV, AA-CP⁴ is a fibrin-binding peptide having at least 80% sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |

In some embodiments of the compound of Formula IV, AA-CP⁴ is a fibrin-binding peptide having at least 80% sequence identity to a polypeptide of:

| SEQ ID | |
|---|---|
| NO: | Sequence |
| 5 | -Y-e-C-Hyp-Y(3-Cl)-G-L-C-H-I-q- |

In some embodiments of the compound of Formula IV, C⁴ is independently selected from the group consisting of:

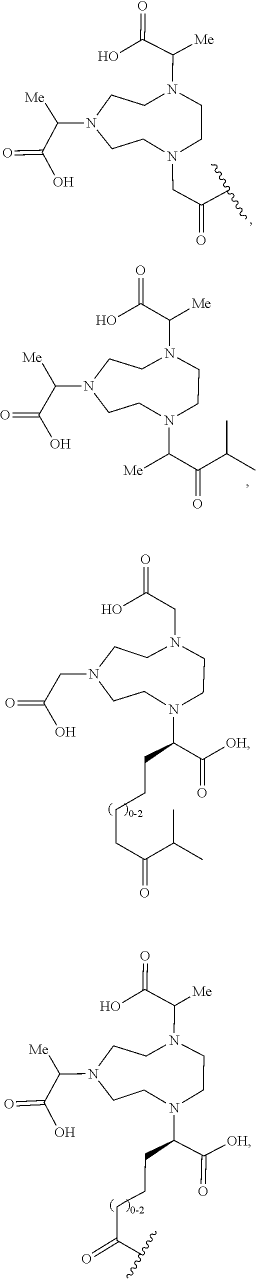

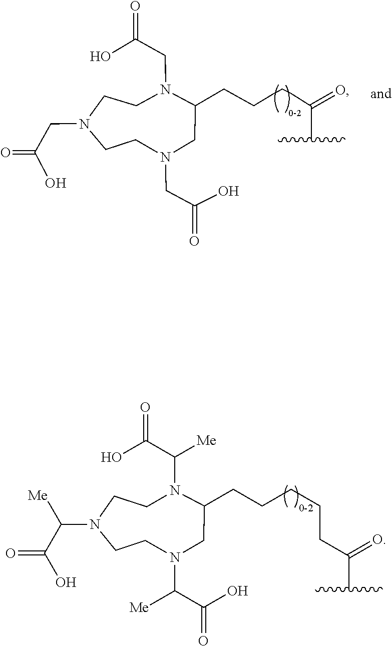

In some embodiments, C⁴ is

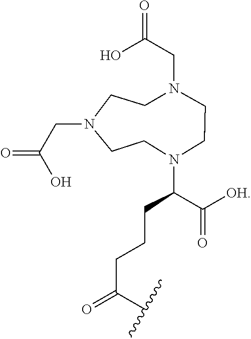 [SMILES: *C(=O)CCC[C@H](C(=O)O)N1CCN(CC(=O)O)CCN(CC(=O)O)CC1]

In some embodiments, C⁴ is independently selected from the group consisting of:

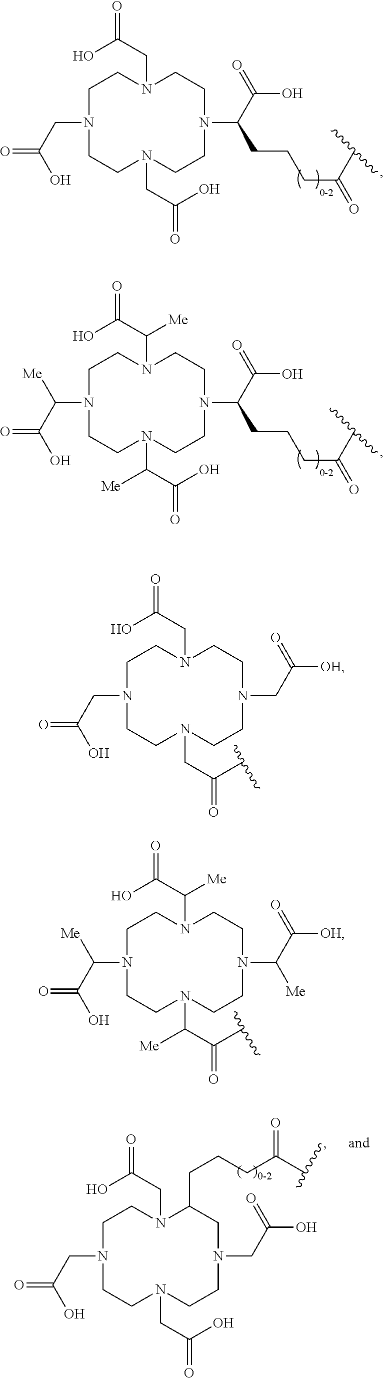

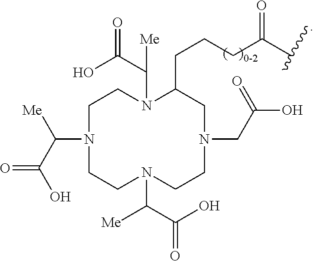

In some embodiments, C⁴ is independently selected from the group consisting of:

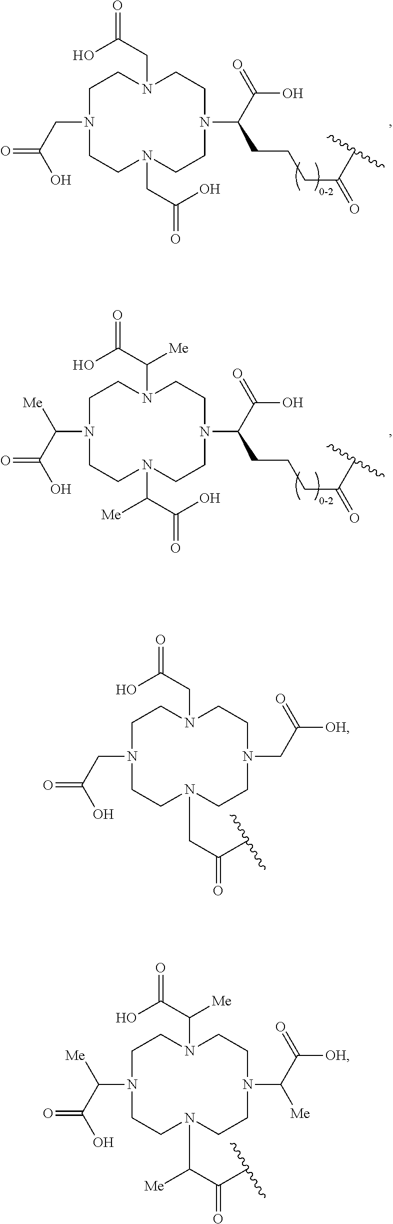

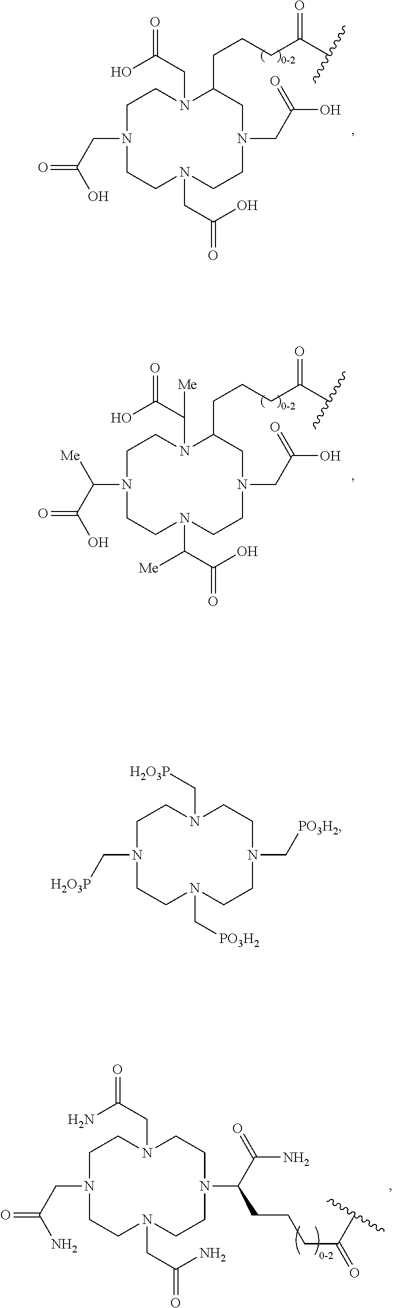

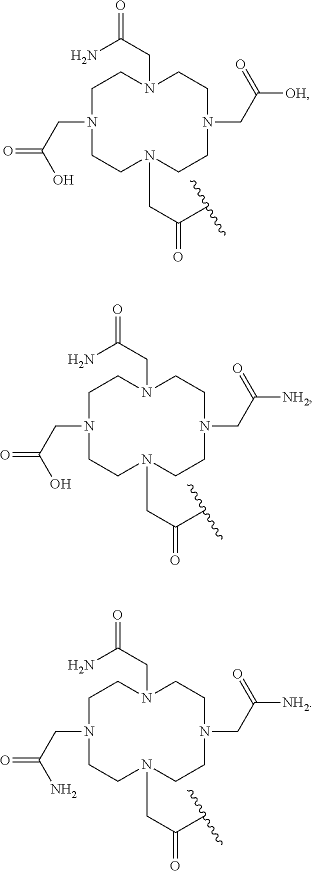 [SMILES: *C(=O)CN1CCN(CC(=O)O)CCN(CC(N)=O)CCN(CC(=O)O)CC1 ; *C(=O)CN1CCN(CC(N)=O)CCN(CC(N)=O)CCN(CC(=O)O)CC1 ; *C(=O)CN1CCN(CC(N)=O)CCN(CC(N)=O)CCN(CC(N)=O)CC1]

In some embodiments of the compound of Formula IV, y is 0. In some embodiments, y is 1.

In some embodiments of the compound of Formula IV, L⁴ is pyridinyl or (pyridinyl)-C(O)—.

In some embodiments of the compound of Formula IV, z is 0. In some embodiments, z is 1.

In some embodiments, y is 1 and z is 0. In some embodiments, y is 1 and z is 1. In some embodiments, 0 and z is 0. In some embodiments, y is 0 and z is 1.

In some embodiments, the compound of Formula IV is a compound of Formula IVa:

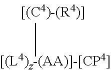

or a pharmaceutically acceptable salt thereof, wherein R⁴ is a radioisotope that is capable of being chelated by the chelating moiety C⁴.

In some embodiments, R⁴ is selected from the group consisting of aluminum-fluoride (Al¹⁸F), scandium-43, scandium-44, scandium-47, manganese-51, manganese-52, copper-60, copper-61, copper-62, copper-64, copper-67, gallium-67, gallium-68, yttrium-86, zirconium-89, technetium-99m, yttrium-90, indium-111, terbium-149, terbium-152, samarium-153, terbium-155, terbium-161, holmium-166, lutetium-177, rhenium-188, lead-203, lead-212, bismuth-213, radium-223, actinium-225, and thorium-227.

In some embodiments, the compound of Formula IV is a compound of Formula IVb:

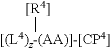

or a pharmaceutically acceptable salt thereof, wherein R⁴ is a radioisotope that is capable of covalently binding to the linker L⁴, the N-terminal amino acid of the fibrin-binding peptide AA, or both.

In some embodiments, R⁴ is selected from the group consisting of fluorine-18, iodine-123, iodine-124, iodine-125, iodine-131, and astatine-211.

In some embodiments, the compound of Formula IV is a compound of Formula IVc:

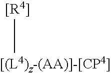

or a pharmaceutically acceptable salt thereof, wherein R⁴ is a radioisotope that is capable of covalently binding to the linker L⁴, the N-terminal amino acid of the fibrin-binding peptide AA, or both.

In some embodiments, R⁴ is selected from the group consisting of fluorine-18, iodine-123, iodine-124, iodine-125, iodine-131, and astatine-211.

In some embodiments, the compound of Formula IV is a compound of Formula IVd:

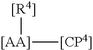

or a pharmaceutically acceptable salt thereof, wherein R⁴ is a radioisotope that is capable of covalently binding to the linker L⁴, the N-terminal amino acid of the fibrin-binding peptide AA, or both.

In some embodiments, R⁴ is selected from the group consisting of fluorine-18, iodine-123, iodine-124, iodine-125, iodine-131, and astatine-211.

In some embodiments, the compound of Formula IV is selected from the group consisting of:

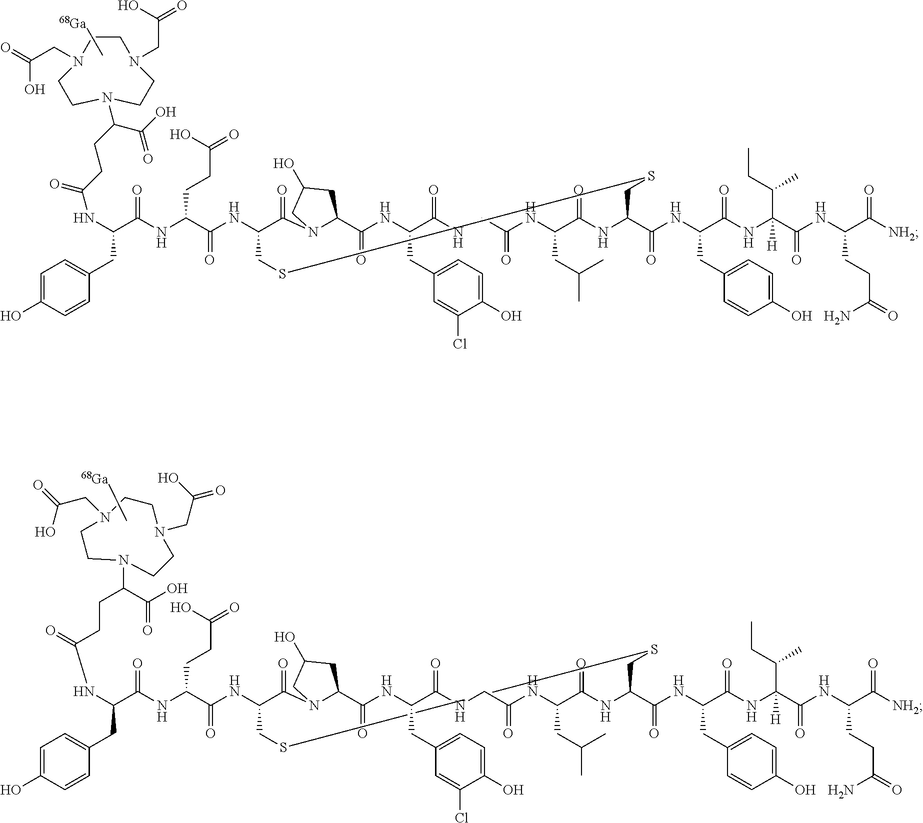

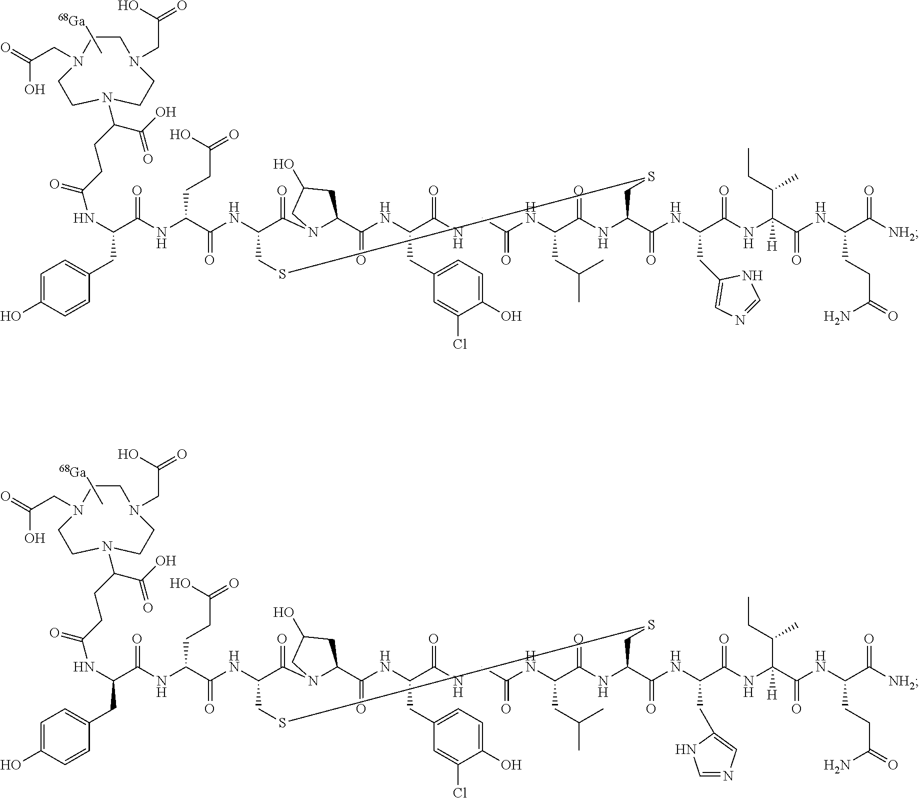

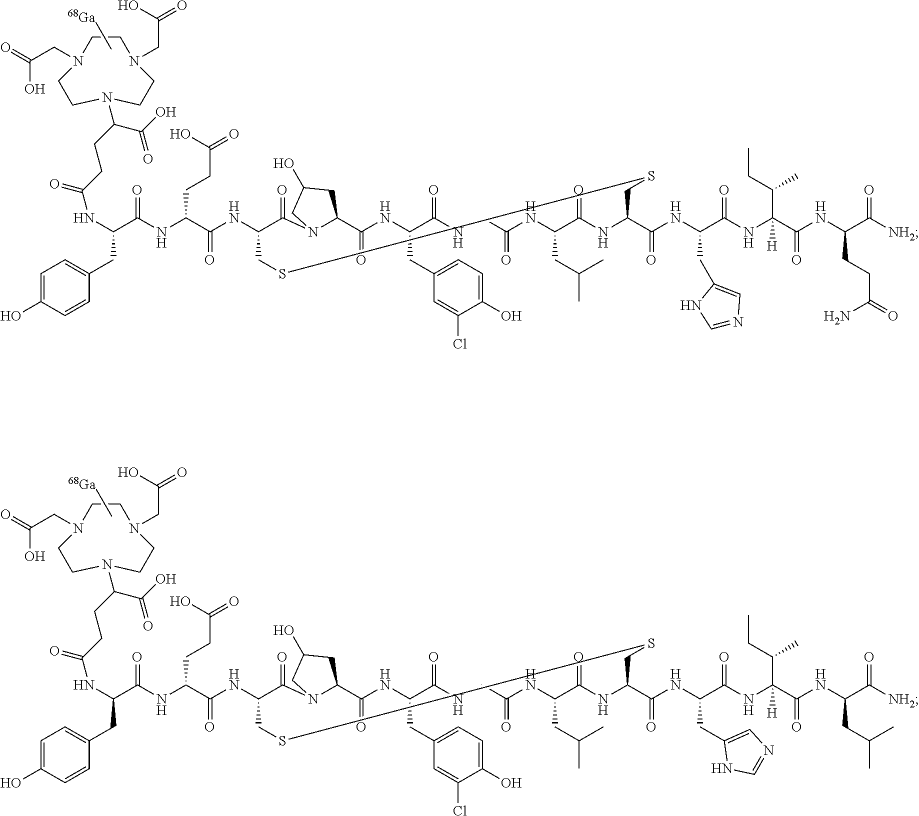

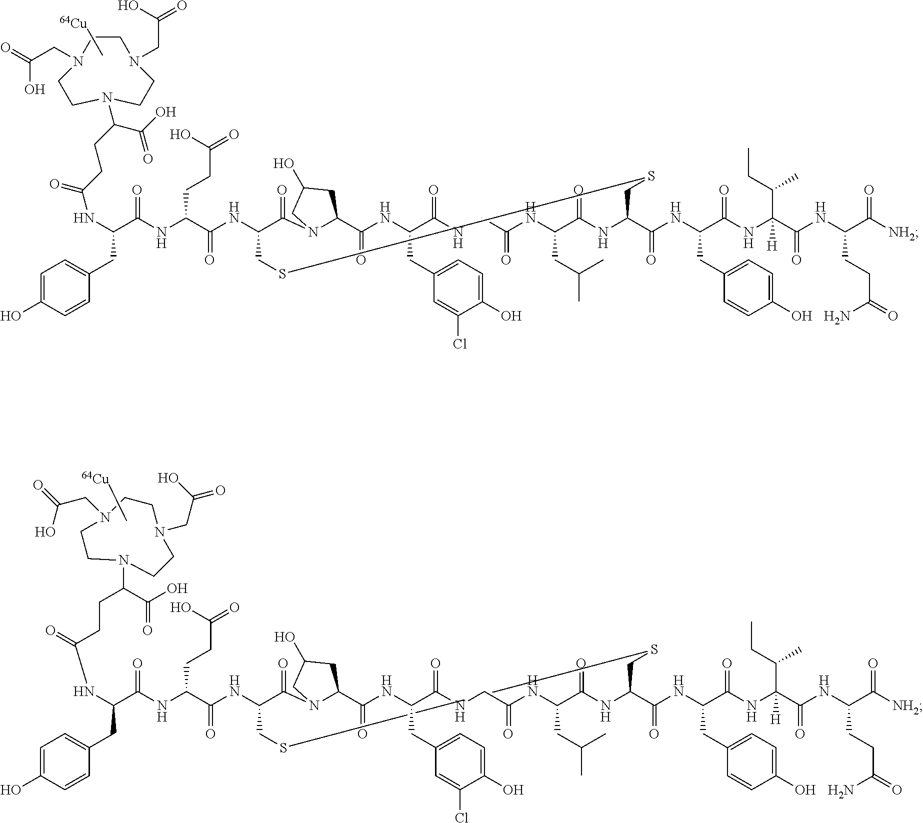

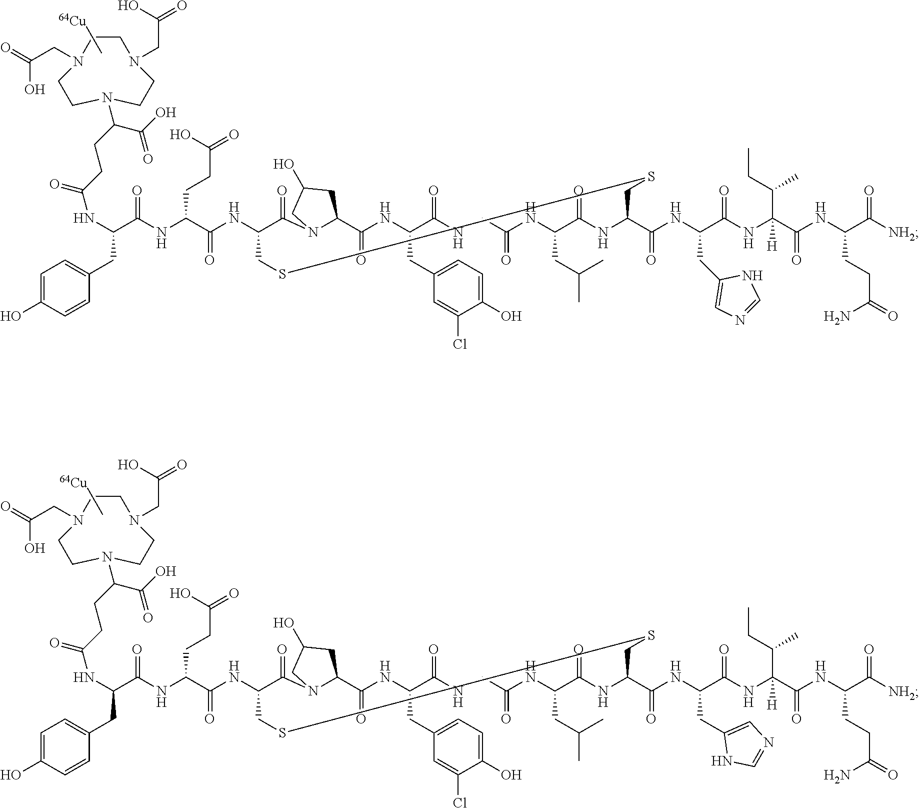

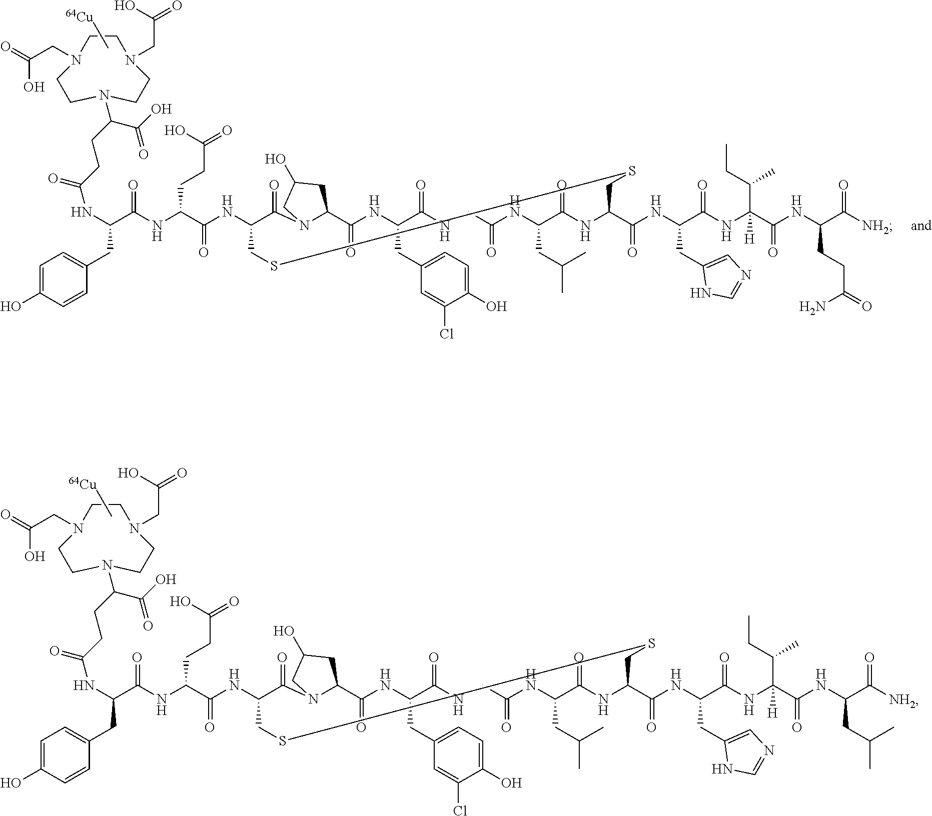

or a pharmaceutically acceptable salt thereof.

In some embodiments, the compound of Formula IV is selected from the group consisting of:

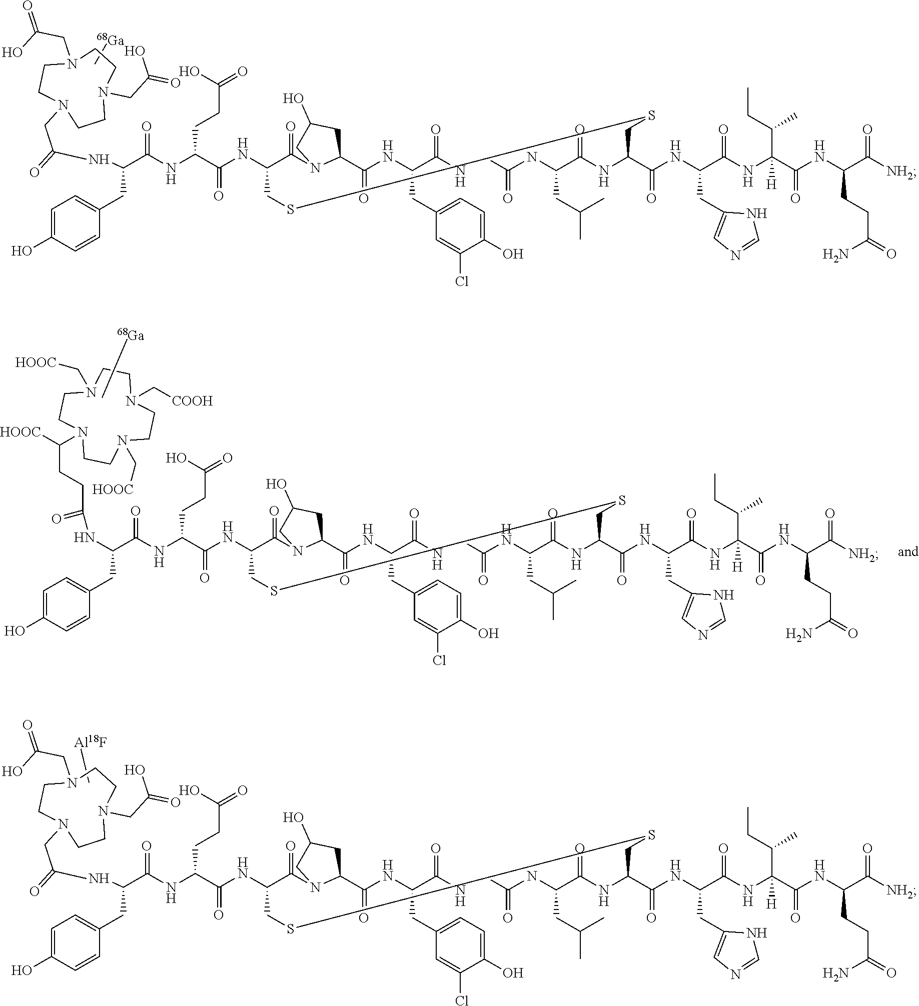

or a pharmaceutically acceptable salt thereof.

In some embodiments, the compound of Formula IV is:

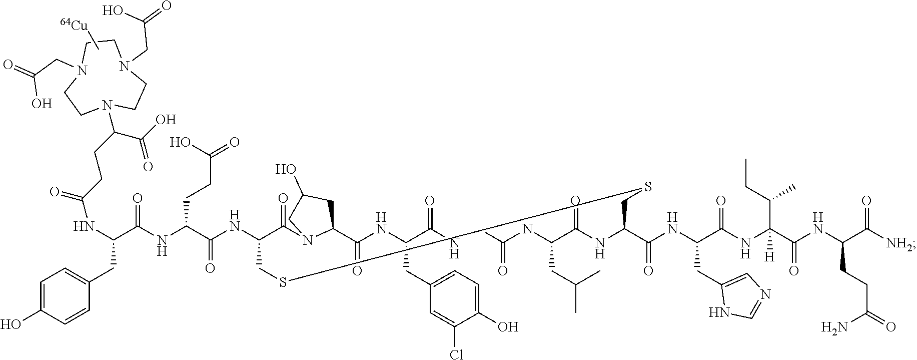

or a pharmaceutically acceptable salt thereof.

In some embodiments, the compound of Formula IV is selected from the group consisting of:

| Com- | | | |
|---|---|---|---|
| pound | | N-terminus | Sequence |
| 25 | | 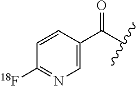 [SMILES: *C(=O)c1ccc([18F])nc1] | -y-e-C-Hyp-Y(3-Cl)-G-L-C-H-I-Q- |
| 26 | | 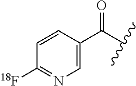 [SMILES: *C(=O)c1ccc([18F])nc1] | -y-e-C-Hyp-Y(3-Cl)-G-L-C-H-I-1- |
| 27 | | 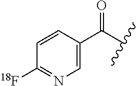 [SMILES: *C(=O)c1ccc([18F])nc1] | -Y-e-C-Hyp-Y(3-Cl)-G-L-C-H-I-Q- |
| 28 | | 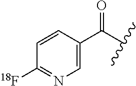 [SMILES: *C(=O)c1ccc([18F])nc1] | -Y-e-C-Hyp-Y(3-Cl)-G-L-C-H-I-q- |
| | 29 | 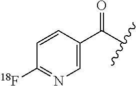 [SMILES: *C(=O)c1ccc([18F])nc1] | -y-e-C-Hyp-Y(3-Cl)-G-L-C-Y-I-Q- |

| | | |
|---|---|---|
| 30 | 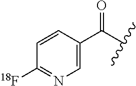 [SMILES: *C(=O)c1ccc([18F])nc1] | -Y-e-C-Hyp-Y(3-Cl)-G-L-C-Y-I-Q- |
| 31 | 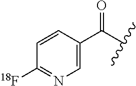 [SMILES: *C(=O)c1ccc([18F])nc1] | -Y-e-C-Hyp-Y(3-Cl)-G-L-C-H-I-1- |
| 32 | 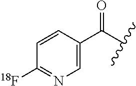 [SMILES: *C(=O)c1ccc([18F])nc1] | -y-e-C-Hyp-Y(3-Cl)-G-L-C-Y-I-1- |
| 33 | 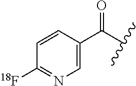 [SMILES: *C(=O)c1ccc([18F])nc1] | -Y-e-C-Hyp-Y(3-Cl)-G-L-C-Y-I-1- |
| 34 | 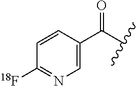 [SMILES: *C(=O)c1ccc([18F])nc1] | -y-e-C-Hyp-Y(3-Cl)-G-L-C-Y-I-q- |
| 35 | 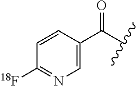 [SMILES: *C(=O)c1ccc([18F])nc1] | -Y-e-C-Hyp-Y(3-Cl)-D-L-C-Y-I-Q- |
| 36 | 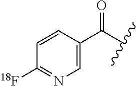 [SMILES: *C(=O)c1ccc([18F])nc1] | -Y-e-C-Hyp-Y(3-Cl)-G-L-C-Y-I-q- |
| 37 | 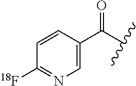 [SMILES: *C(=O)c1ccc([18F])nc1] | -Y-e-C-Hyp-Y(3-Cl)-d-L-C-Y-I-Q- |
| 38 | 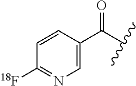 [SMILES: *C(=O)c1ccc([18F])nc1] | -Y-h-C-Hyp-Y(3-Cl)-G-L-C-Y-I-Q- |

Also provided in the present disclosure is a pharmaceutical composition comprising a compound of Formula IV, or a pharmaceutically acceptable salt thereof, and a pharmaceutically acceptable excipient.

In some embodiments, the pharmaceutical composition comprises a radical scavenger. In some embodiments, the radical scavenger is an antioxidant. In some embodiments, the radical scavenger is selected from the group consisting of carnosic acid, green tea extract, apigenin, diosmine, rosmarinic acid, lipoic acid, beta carotene, L-ascorbic acid (vitamin C), N-acetyl cysteine (NAC), δ-tocopherol, rutin, amifostine, resveratrol, gentisic acid, and gallic acid.

Also provided is a method of imaging fibrin in a mammal, the method comprising: a) administering to the mammal an effective amount of a pharmaceutical composition containing the compound of Formula IV or an effective amount of a compound of Formula IV, or a pharmaceutically acceptable salt thereof, wherein R⁴ is a radioisotope capable of detection using a nuclear imaging technique; b) acquiring an image of the fibrin of the mammal using a nuclear imaging technique; c) acquiring an anatomical image of the mammal using magnetic resonance imaging or computed tomography; and d) overlaying the images of steps b) and c) to localize the image of fibrin within the anatomical image of the mammal.

In some embodiments, the presence of the fibrin is associated with neuroinflammation. In some embodiments, the neuroinflammation is associated with Alzheimer's disease, multiple sclerosis, or traumatic brain injury.

In some embodiments, the method further comprises: e) administering an effective amount of a pharmaceutical composition containing a compound of Formula IV or an effective amount of a compound of Formula IV, or a pharmaceutically acceptable salt thereof, wherein R⁴ is a therapeutic radioisotope.

In some embodiments of the method, the fibrin is present in a tumor. In some embodiments, the tumor is cancerous. In some embodiments, the fibrin is present in a thrombus.

Also provided in the present disclosure is a method of treating a disease or condition associated with the presence of fibrin in a mammal, the method comprising: administering to the mammal an effective amount of a pharmaceutical composition containing a compound of Formula IV or an effective amount of a compound of Formula IV, or a pharmaceutically acceptable salt thereof, wherein R⁴ is a therapeutic radioisotope.

In some embodiments, the method further comprises administering an amino acid solution. In some embodiments, the amino acid solution comprises L-lysine, L-arginine, and pharmaceutically acceptable salts thereof, and combinations thereof. In some embodiments, the amino acid solution comprises L-lysine HCl and L-arginine HCl. in some embodiments, the amino acid solution is administered prior to, concomitantly, after, or combinations thereof, administering a pharmaceutical composition containing a compound of Formula IV or an effective amount of a compound of Formula IV, or a pharmaceutically acceptable salt thereof. In some embodiments, the amino acid solution is administered about 30 minutes prior to administering a pharmaceutical composition containing a compound of Formula IV or an effective amount of a compound of Formula IV, or a pharmaceutically acceptable salt thereof. In some embodiments, the amino acid solution is administered concomitantly with administering a pharmaceutical composition containing a compound of Formula IV or an effective amount of a compound of Formula IV, or a pharmaceutically acceptable salt thereof. In some embodiments, the amino acid solution is administered about 30 minutes after administering a pharmaceutical composition containing a compound of Formula IV or an effective amount of a compound of Formula IV, or a pharmaceutically acceptable salt thereof.

In some embodiments, the method further comprises administering an antiemetic agent. In some embodiments, the antiemetic agent is administered prior to, concomitantly, after, or combinations thereof, administering a pharmaceutical composition containing a compound of Formula IV or an effective amount of a compound of Formula IV, or a pharmaceutically acceptable salt thereof. In some embodiments, the antiemetic agent is administered prior to, concomitantly, after, or combinations thereof, administering the amino acid solution. In some embodiments, the antiemetic agent is selected from the group consisting of 5-HT₃ receptor antagonists, corticosteroids, neurokinin-1 (NK-1) receptor inhibitors, prochlorperazine, metoclorpramide, and cannabinoids.

In some embodiments of the method, the disease or condition associated with the presence of fibrin is cancer.

Also provided in the present disclosure is a method of treating cancer in a mammal, the method comprising: administering to the mammal an effective amount of a pharmaceutical composition containing a compound of Formula IV or an effective amount of a compound of Formula IV, or a pharmaceutically acceptable salt thereof, wherein R⁴ is a therapeutic radioisotope. In some embodiments, the R⁴ that is a therapeutic radioisotope is selected from the group consisting of scandium-47, copper-67, yttrium-90, iodine-131, samarium-153, terbium-161, holmium-166, lutetium-177, rhenium-188, astatine-211, lead-212, bismuth-213, radium-223, actinium-225, and thorium-227. In some embodiments, the R⁴ that is a therapeutic radioisotope is selected from the group consisting of yttrium-90, lutetium-177, and actinium-225. In some embodiments, the R⁴ that is a therapeutic radioisotope is yttrium-90. In some embodiments, the R⁴ that is a therapeutic radioisotope is lutetium-177. In some embodiments, the R⁴ that is a therapeutic radioisotope is actinium-225.

Also provided is a method of detecting and treating a disease or condition associated with the presence of fibrin in a mammal, the method comprising: a) administering to the mammal an effective amount of a pharmaceutical composition containing a compound of Formula IV or an effective amount of a compound of Formula IV, or a pharmaceutically acceptable salt thereof, wherein R⁴ is a radioisotope capable of detection using a nuclear imaging technique; b) acquiring an image of the fibrin of the mammal using a nuclear imaging technique; c) acquiring an anatomical image of the mammal using magnetic resonance imaging or computed tomography; d) overlaying the images of steps b) and c) to localize the image of fibrin within the anatomical image of the mammal; and e) administering an effective amount of a pharmaceutical composition containing a compound of Formula IV or an effective amount of a compound of Formula IV, or a pharmaceutically acceptable salt thereof, wherein R⁴ is a therapeutic radioisotope.

In some embodiments of the method, the R⁴ that is a radioisotope capable of detection using a nuclear imaging technique is selected from the group consisting of fluorine-18, aluminum fluoride (Al¹⁸F), scandium-43, scandium-44, copper-64, gallium-68, yttrium-86, zirconium-89, indium-111, iodine-123, iodine-124, terbium-149, terbium-152, terbium-155, and lead-203. In some embodiments, the R⁴ that is capable of detection using a nuclear imaging technique is selected from the group consisting of fluorine-18, copper-64, and gallium-68. In some embodiments the R⁴ that is capable of detection using a nuclear imaging technique is fluorine-18. In some embodiments, the R⁴ that is capable of detection using a nuclear imaging technique is copper-64. In some embodiments, the R⁴ that is capable of detection using a nuclear imaging technique is gallium-68.

In some embodiments of the method, the R⁴ that is a therapeutic radioisotope is selected from the group consisting of scandium-47, copper-67, yttrium-90, iodine-131, samarium-153, terbium-161, holmium-166, lutetium-177, rhenium-188, astatine-211, lead-212, bismuth-213, radium-223, actinium-225, and thorium-227. In some embodiments, the R⁴ that is a therapeutic radioisotope is selected from the group consisting of yttrium-90, lutetium-177, and actinium-225. In some embodiments, the R⁴ that is a therapeutic radioisotope is yttrium-90. In some embodiments, the R⁴ that is a therapeutic radioisotope is lutetium-177. In some embodiments, the R⁴ that is a therapeutic radioisotope is actinium-225.

In some embodiments, the method further comprises administering an amino acid solution. In some embodiments, the amino acid solution is administered prior to, concomitantly, after, or combinations thereof, administering a pharmaceutical composition containing a compound of Formula IV or an effective amount of a compound of Formula IV, or a pharmaceutically acceptable salt thereof.

In some embodiments, the method further comprises administering an antiemetic agent. In some embodiments, the antiemetic agent is administered prior to, concomitantly, after, or combinations thereof, administering the amino acid solution.

In some embodiments of the method, the disease or condition associated with the presence of fibrin is cancer.

Also provided in the present disclosure is a method of detecting and treating a disease or condition associated with the presence of fibrin in a mammal, the method comprising: a) administering to the mammal an effective amount of a pharmaceutical composition containing a compound of Formula IV or an effective amount of a compound of Formula IV, or a pharmaceutically acceptable salt thereof, where R⁴ is a radioisotope capable of detection using a nuclear imaging technique selected from the group consisting of fluorine-18, copper-64, and gallium-68; b) acquiring an image of the fibrin of the mammal using a nuclear imaging technique; c) acquiring an anatomical image of the mammal using magnetic resonance imaging or computed tomography; d) overlaying the images of steps b) and c) to localize the image of fibrin within the anatomical image of the mammal; and e) administering an effective amount of a pharmaceutical composition containing a compound of Formula IV or an effective amount of a compound of Formula IV, or a pharmaceutically acceptable salt thereof, where R⁴ is a therapeutic radioisotope selected from the group consisting of yttrium-90, lutetium-177, and actinium-225. In some embodiments, the fibrin is present in a tumor. In some embodiments, the tumor is cancerous.

Also provided in the present disclosure is a compound of Formula V:

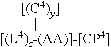

or a pharmaceutically acceptable salt thereof, wherein:
   C⁴ is a chelating moiety;
   CP⁴ is a fibrin-binding peptide;
   AA is the N-terminal amino acid of the fibrin-binding peptide;
   L⁴ is a linker;
   y is an integer selected from 0 or 1; and
   z is an integer selected from 0 or 1.

In some embodiments, the compound of Formula V is a compound selected from the group consisting of:

| Compound | Sequence |
|---|---|
| 16 | NODAGA-Y-e-C-Hyp-Y(3-Cl)-G-L-C-Y-I-Q-NH₂ |
| 17 | NODAGA-y-e-C-Hyp-Y(3-Cl)-G-L-C-Y-I-Q-NH₂ |
| 18 | NODAGA-Y-e-C-Hyp-Y(3-Cl)-G-L-C-H-I-Q-NH₂ |
| 19 | NODAGA-y-e-C-Hyp-Y(3-Cl)-G-L-C-H-I-Q-NH₂ |
| 20 | NODAGA-Y-e-C-Hyp-Y(3-Cl)-G-L-C-H-I-q-NH₂ |
| 21 | NODAGA-y-e-C-Hyp-Y(3-Cl)-G-L-C-H-I-I-NH₂ |
| 22 | NODAGA-y-e-c-Hyp-Y(3-Cl)-G-L-C-H-I-q-NH₂ |
| 23 | DOTAGA-Y-e-C-Hyp-Y(3-Cl)-G-L-C-H-I-q-NH₂ |
| 24 | NOTA-Y-e-C-Hyp-Y(3-Cl)-G-L-C-H-I-q-NH₂ |

Provided herein are compounds of Formula I:

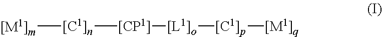

or a pharmaceutically acceptable salt thereof,
   wherein each M¹ is independently copper-64 or gallium-68;
   each C¹ is a chelating moiety independently selected from the group consisting of:

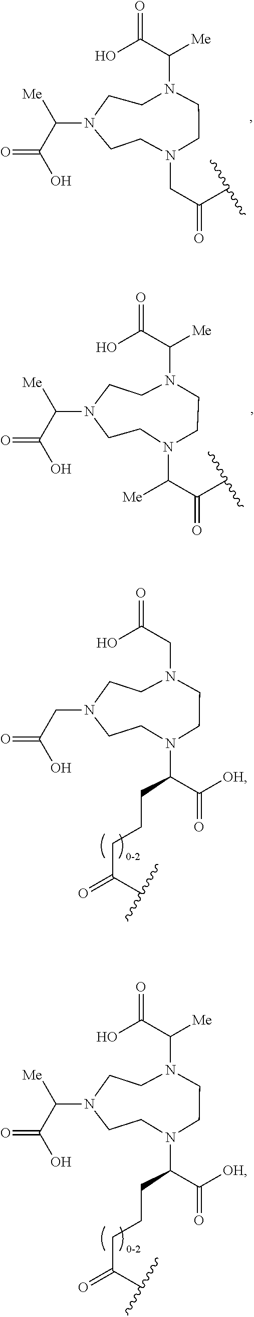

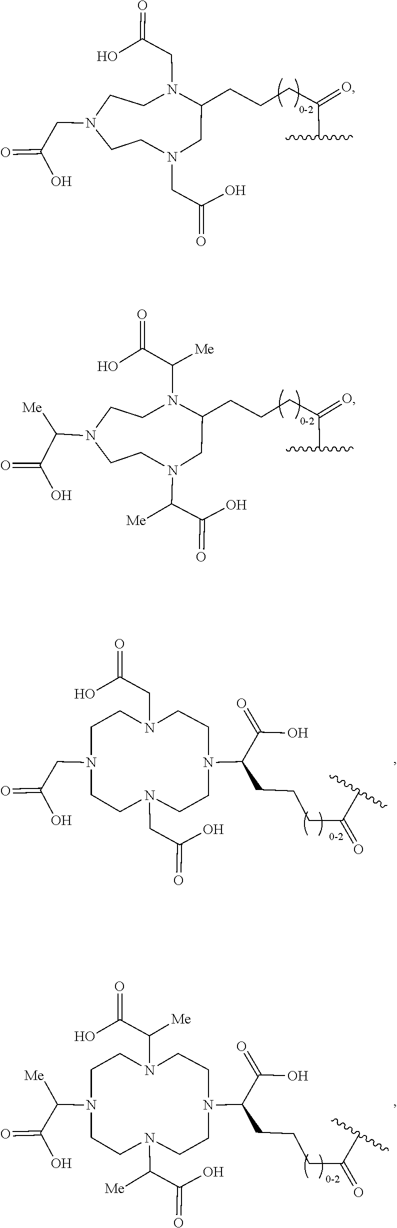

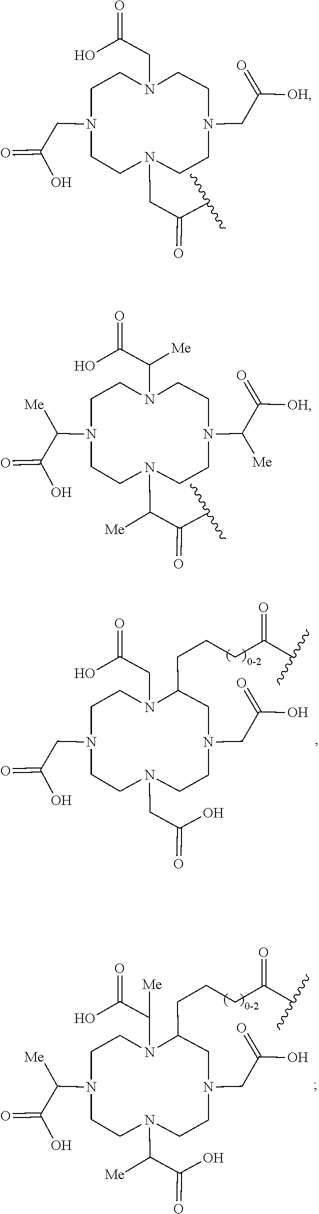

CP¹ is a fibrin-binding peptide;
each L¹ is independently a linker moiety;
m is an integer selected from 0 to 5;
n is an integer selected from 0 to 5;
is an integer selected from 0 to 5;
p is an integer selected from 0 to 5; and
q is an integer selected from 0 to 5.

In some embodiments, M¹ is copper-64. In some embodiments, M¹ is gallium-68.

In some embodiments, each C¹ is independently selected from the group consisting of:

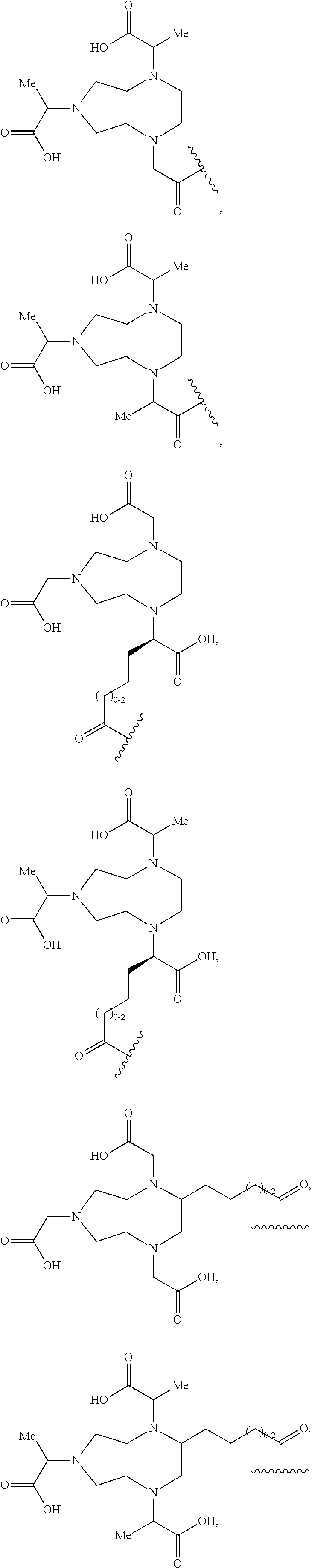

In some embodiments, each C¹ is

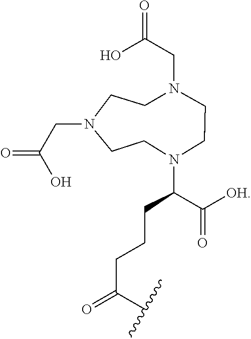 [SMILES: *C(=O)CCC[C@H](C(=O)O)N1CCN(CC(=O)O)CCN(CC(=O)O)CC1]

In some embodiments, each C¹ is independently selected from the group consisting of:

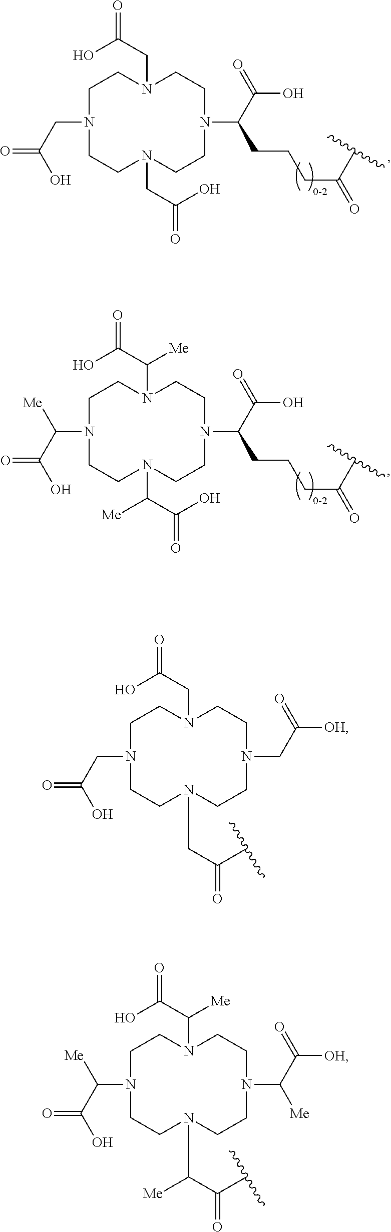

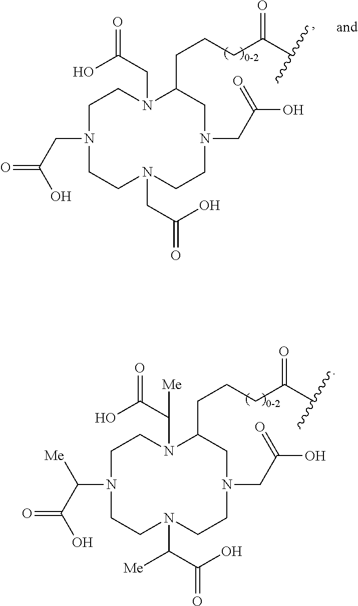

In some embodiments, CP¹ is a fibrin-binding peptide comprising a sequence having at least 80% sequence identity to the polypeptide of SEQ ID NO: 1:

(SEQ ID NO: 1)
-y*-X¹-C-Hyp-Y(3-Cl)-X²-L-C-X³-I-X⁴-

wherein each of X¹, X², X³, and X⁴ is independently any amino acid; and
y* is L-tyrosine or D-tyrosine.

In some embodiment, CP¹ is a fibrin-binding peptide comprising a polypeptide having at least 80% sequence to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments, CP¹ is a fibrin-binding peptide having at least 80% sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments, each L¹ is independently selected from the group consisting of:

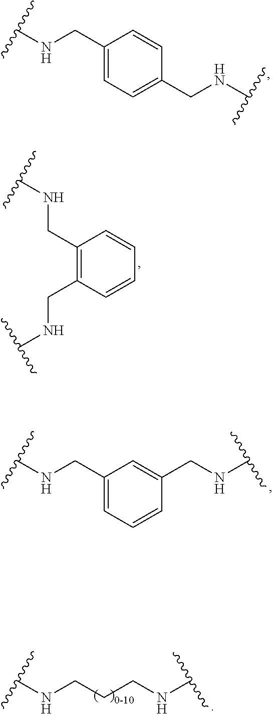

In some embodiments, m is 1. In some embodiments, p is 1. In some embodiments, n is 1. In some embodiments, o is 1.

In some embodiments, the compound of Formula I is selected from the group consisting of:

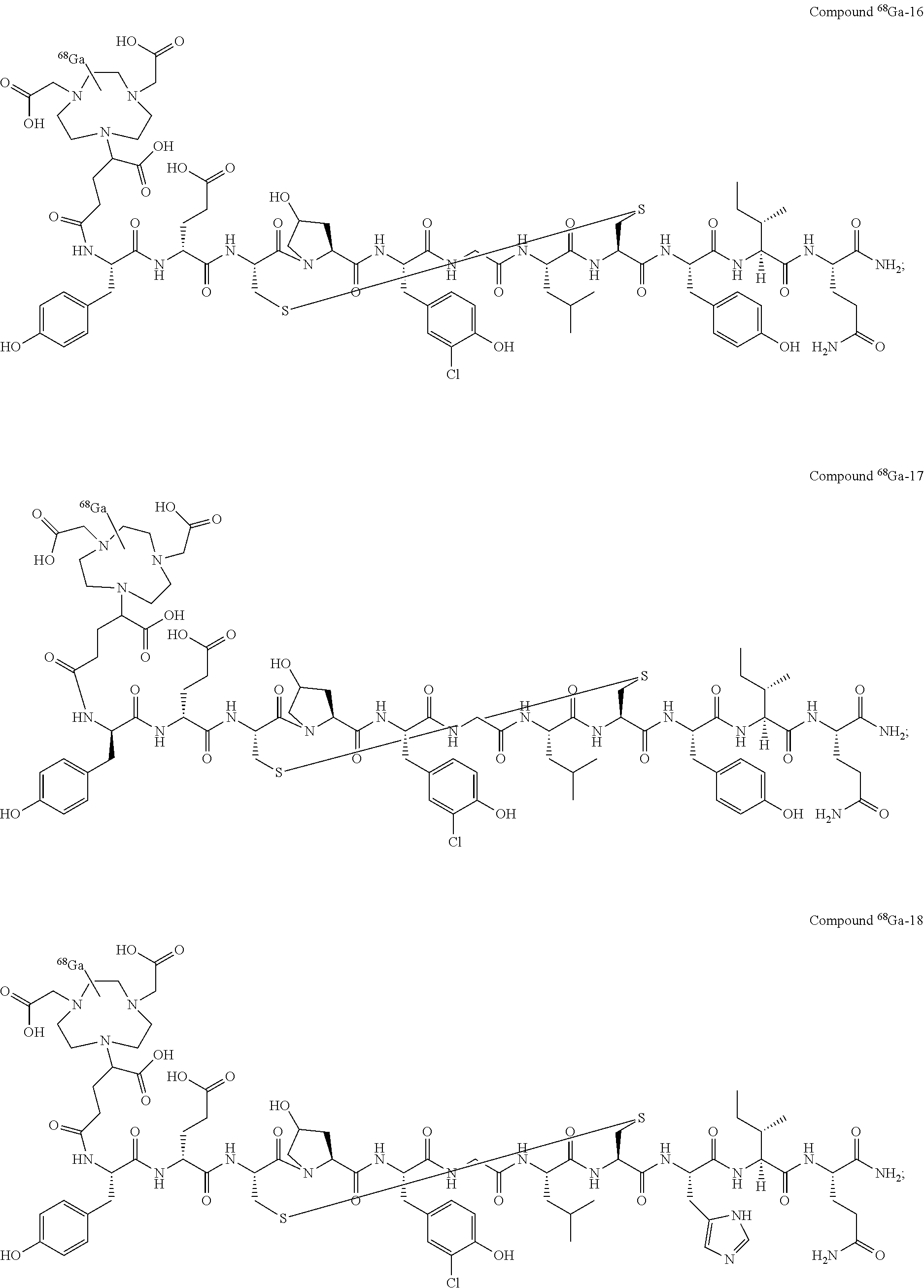

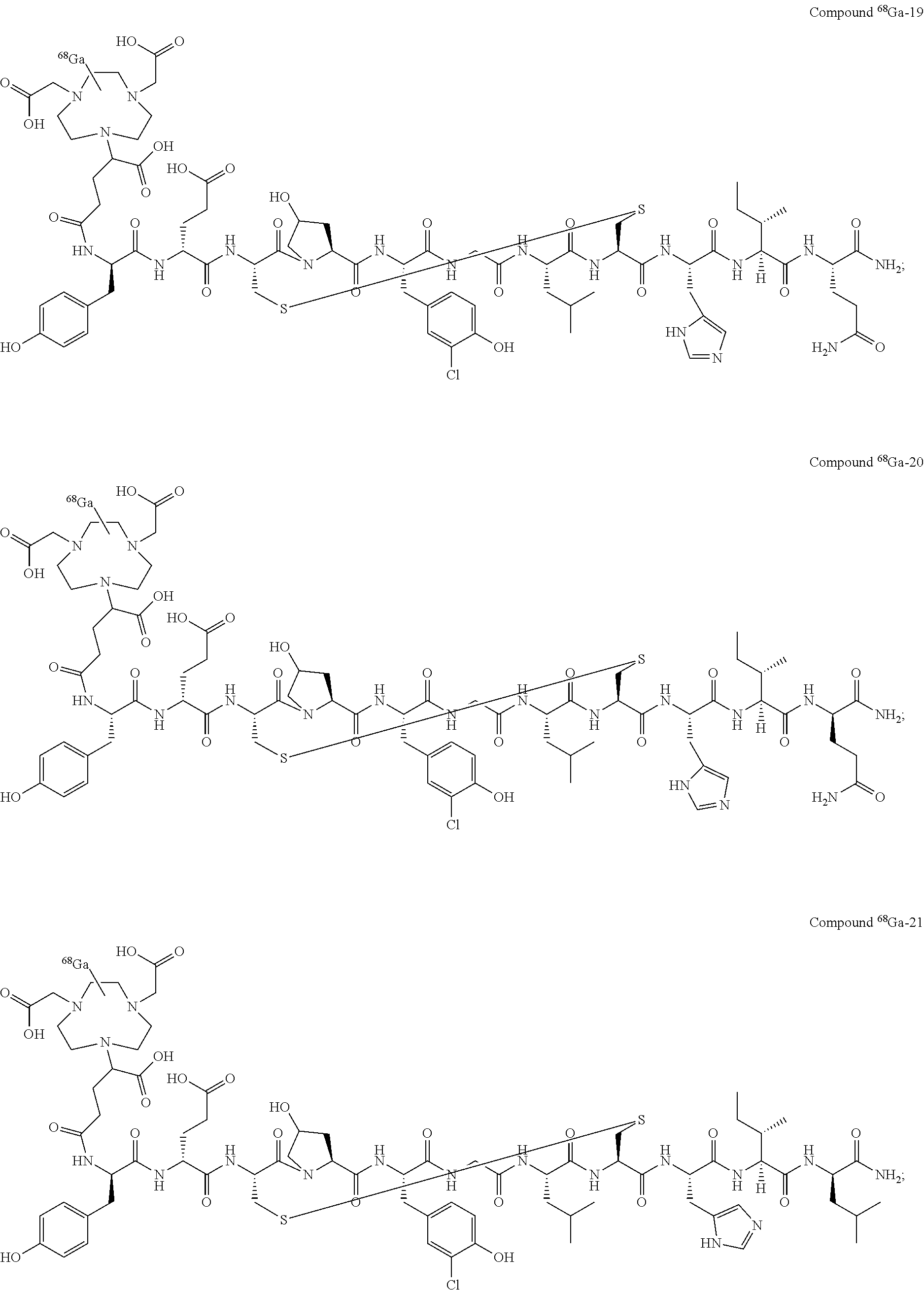

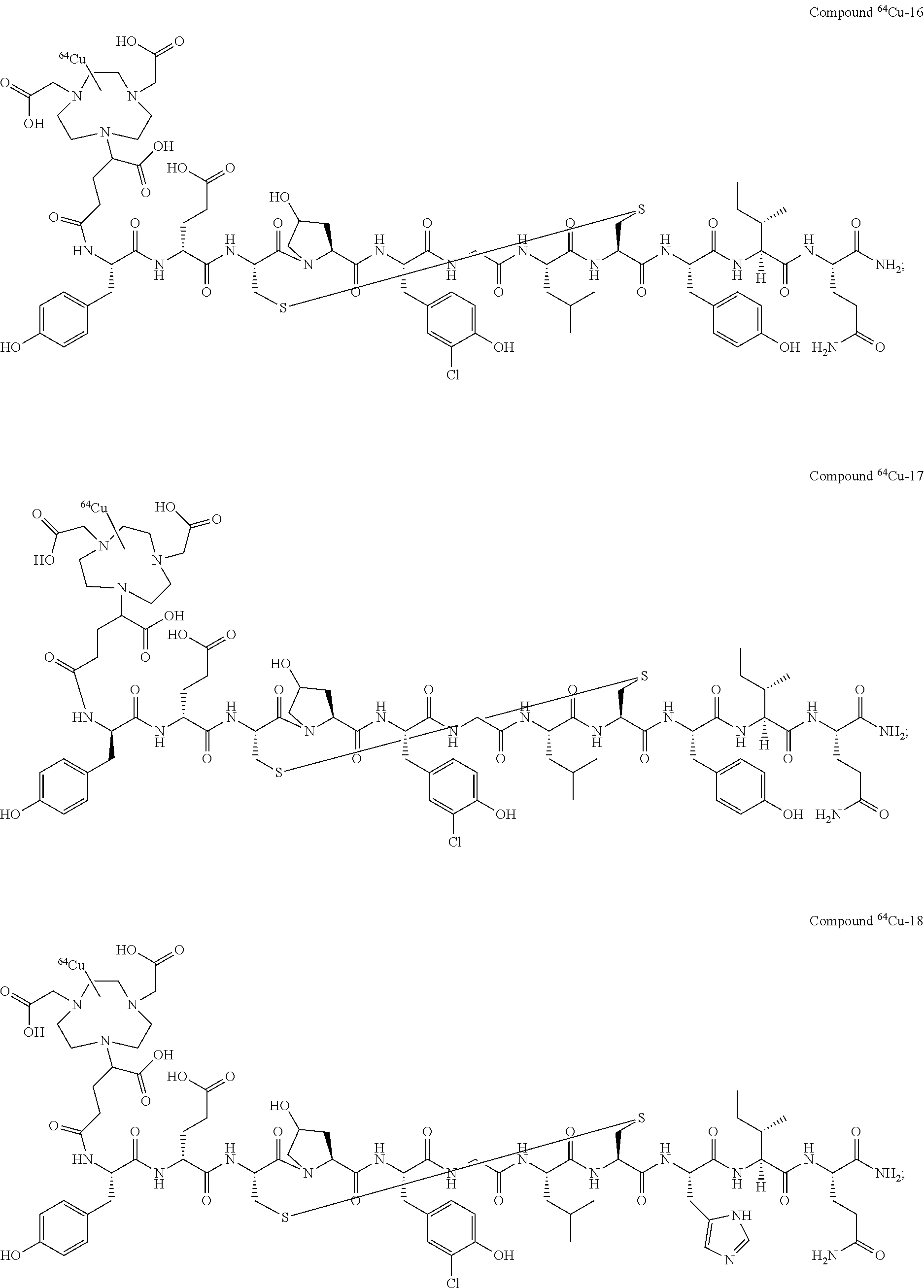

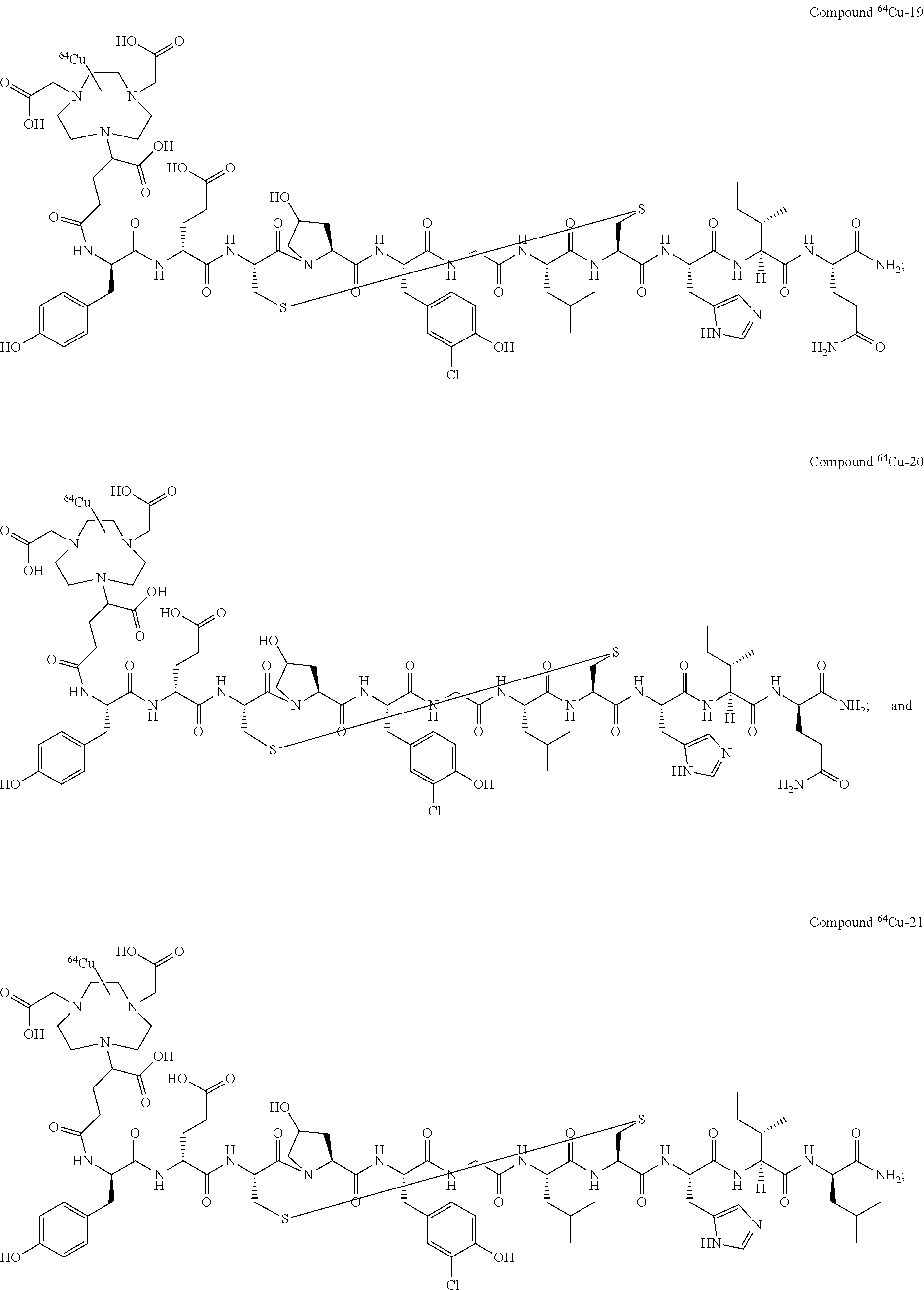

or a pharmaceutically acceptable salt thereof.

Also provided herein are compounds of Formula II:

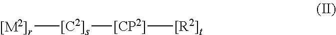

or a pharmaceutically acceptable salt thereof,
   wherein each M² is independently actinium-225, astatine-211, bismuth-213, copper-64, copper-67, aluminum fluoride (Al¹⁸F), gallium-68, holmium-166, indium-111, iodine-123, iodine-124, and iodine-131, lead-203, lead-212, lutetium-177, radium-223, samarium-153, scandium-43, scandium-44, scandium-47, terbium-149, terbium-152, terbium-155, terbium-161, thorium-227; yttrium-86, yttrium-90, or zirconium-89;
   each C² is independently a chelating moiety;
   CP² is a fibrin-binding peptide;
   each R² is independently an organic, non-chelating moiety;
   r is an integer selected from 0 to 5;
   s in an integer selected from 0 to 5; and
   t is an integer selected from 0 to 5.

In some embodiments, M² is copper-64. In some embodiments, M² is gallium-68.

In some embodiments, CP² is a fibrin-binding peptide comprising a polypeptide having at least 80% sequence identity to the polypeptide of SEQ ID NO: 16:

(SEQ ID NO: 16)
-y*-X⁵-C-Hyp-Y(3-Cl)-X⁶-L-C-X⁷-I-X⁸-

wherein each of X⁵, X⁶, X⁷, and X⁸ is independently any amino acid; and
y* is L-tyrosine or D-tyrosine.

In some embodiments, CP² is a fibrin-binding peptide comprising a polypeptide having at least 80% sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-c-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments, CP² is a fibrin-binding peptide having at least 80% sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments, r is 1. In some embodiments, s is 1. In some embodiments, t is 1.

Also provided herein are compounds of Formula III:

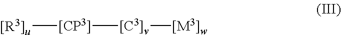

or a pharmaceutically acceptable salt thereof,
   wherein each M³ is independently copper-64 or gallium-68;
   each C³ is a chelating moiety;
   CP³ is a fibrin-binding peptide;
   each R³ is independently an organic, non-chelating moiety;
   u is an integer selected from 0 to 5;
   v in an integer selected from 0 to 5; and
   w is an integer selected from 0 to 5.

In some embodiments, M³ is copper-64. In some embodiments, M³ is gallium-68.

In some embodiments, CP³ is a fibrin-binding peptide comprising a polypeptide having at least 80% sequence identity to the polypeptide of SEQ ID NO:17:

(SEQ ID NO: 17)
-y*-X⁹-C-Hyp-Y(3-Cl)-X¹⁰-L-C-X¹¹-I-X¹²-

wherein each of X⁹, X¹⁰, X¹¹, and X¹² is independently any amino acid; and
y* is L-tyrosine or D-tyrosine.

In some embodiments, CP³ is a fibrin-binding peptide comprising a sequence having at least 80% sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments, CP³ is a fibrin-binding peptide having at least 80% sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments, u is 1. In some embodiments, v is 1. In some embodiments, w is 1.

Also provided herein are pharmaceutical compositions comprising a compound of Formula I, Formula II, or Formula III, or a pharmaceutically acceptable salt thereof, and a pharmaceutically acceptable excipient.

Also provided herein are methods for imaging fibrin in a mammal, the method comprising administering to the mammal an effective amount of a compound of Formula I, Formula II, or Formula III, or a pharmaceutically acceptable salt thereof, or a pharmaceutical composition comprising a compound of Formula I, Formula II, or Formula III, or a pharmaceutically acceptable salt thereof, and a pharmaceutically acceptable excipient; acquiring an image of the fibrin of the mammal using a nuclear imaging technique; acquiring an anatomical image of the mammal using magnetic resonance imaging or computed tomography; and overlaying the images to localize the image of the fibrin within the anatomical image of the mammal.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

Other features and advantages of the invention will be apparent from the following detailed description and figures, and from the claims.

### DESCRIPTION OF DRAWINGS

FIG. 1 depicts a Fluorescence Polarization DD(E) Binding/Displacement assay for Compounds 4, 6, 7, 10, 12, 13, 14, and 15.

FIG. 2 depicts a Fluorescence Polarization DD(E) Binding/Displacement assay for Compounds 2, 3, 5, 8, 9, and 11.

FIG. 3 depicts the compound stability in rat plasma at 37° C. up to 4 hours.

FIG. 4 depicts the radio HPLC trace of ¹⁸F-Py-TFP following semi-preparative HPLC purification.

FIG. 5 depicts the radio HPLC trace of Compound ¹⁸F-7 following semi-preparative HPLC purification.

FIG. 6 depicts data from the plate assay plotted on a linear scale to highlight saturation of binding sites at about 2 per fibrin monomer in TBS buffer (top) and in human plasma (bottom).

FIG. 7 depicts Compound ¹⁸F-7 binding to human fibrin in TBS buffer (top, K_{d}=1.6±0.2 μM) and in human plasma (bottom, K_{d}=1.8±0.2 μM).

FIG. 8 depicts the radio HPLC trace for Compound ⁶⁸Ga-20.

FIG. 9 depicts the blood clearance in a rat model of carotid artery crush injury. Blood samples were drawn prior to probe injection and at 2, 5, 10, 15, 30, 60, 90, 120 min post injection. Activity in each sample was calculated as percent injected dose (% ID) per gram of tissue.

FIG. 10 depicts the percentage of ⁶⁸Ga-labeled compounds bound to fibrin before (T=0 min) and after (T=10, 60) rat injection.

FIG. 11 depicts the fraction of circulating radioactivity. Blood collected at 10 and 60 min post probe injection were centrifuged to separate plasma. Blood plasma was then incubated in fibrin immobilized wells for 2 hours at RT. After incubation, the counts in the supernatant in both the fibrin-containing and empty wells were measured on a gamma counter and divided by the weight of plasma to determine the concentration of unbound probe, [unbound], and total probe, [total], respectively. The amount of ⁶⁸Ga containing species bound to fibrin, [bound], was calculated from [bound]=[total]−[unbound]. As a positive control, an aliquot of the dose was spiked into blood plasma and used to estimate the total possible fibrin binding in the assay (% bound at t=0). The amount of functional probe in the blood at time t was determined by taking the ratio of the % bound to fibrin at time t compared to the % bound at t=0, and multiplying this ratio by the measured total ⁶⁸Ga % ID/g in the blood.

FIG. 12 depicts the radio HPLC traces for blood analysis following injection of Compounds ⁶⁸Ga-16, ⁶⁸Ga-17, ⁶⁸Ga-18, ⁶⁸Ga-19, ⁶⁸Ga-20, and ⁶⁸Ga-21 in a rat. The traces represent the blood analysis 15 and 90 min after injection.

FIG. 13 depicts bio-distribution in rats after injection of ⁶⁸Ga-labeled compounds. The activity in various organs are shown as percent injected dose (% ID) per gram of tissue.

FIG. 14 depicts representative images of autoradiography showing Compounds ⁶⁸Ga-19 (left) and ⁶⁸Ga-20 (right) activity in a clot (ipsi), as compared to the contra-lateral side of the subject.

FIG. 15 (top panel) depicts orthogonal CT images of rats with a thrombus in the right carotid artery. Yellow arrowheads show the location of the right carotid, which is slightly hyperintense due to CT contrast infusion. The orange arrowhead in axial image (top left) shows the contralateral carotid. (Bottom panel) PET-CT fusion images after administration of ⁶⁸Ga-20 with the PET images rendered in color scale. The green crosshairs indicate the location of the three orthogonal image slices shown, which in this case, are centered on the thrombus in the right carotid. In this animal model, the thrombus is induced by making an incision in the throat, isolating the right common carotid, and then generating a crush injury to the vessel. This model also results in microthrombosis around the site of surgical injury and this is denoted by the red arrow in the axial (bottom left) and sagittal (bottom right) images.

FIG. 16 depicts the PET uptake of Compound ⁶⁸Ga-20 over time in control rabbits and plaque-rupture rabbits. The black solid line represents the rupture:control ratio at each time point.

FIG. 17 depicts representative images of the Compound ⁶⁸Ga-20 PET (top panel), high resolution T2 MR (middle panel), and TOF (bottom panel) from the plaque-rupture rabbits (left panel) and control rabbits (right panel). Arrows indicate the abdominal aorta (green) and inferior vena cava (blue). Insert panels shows zoom PET-MR image of the aorta and vena cava.

FIG. 18 shows that the uptake of the fibrin-binding probe ⁶⁸Ga-20 was significantly higher than the non-binding probe ⁶⁸Ga-22 in specimens from carotid endarterectomy patients. Representative autoradiography (FIGS. 18A-18B and 18G-18H) and light microscopy images of Carstairs' stained sections (FIGS. 18C-18F and 18I-18L) from patient specimens with high (FIGS. 18A-18F) and low (FIGS. 18G-18L)⁶⁸Ga-20 uptake. While high-uptake specimens (FIGS. 18C-18F) displayed an intense presence of fibrin (yellow arrows) with or without accompanying erythrocytes (green arrows), even forming fibrin meshes (FIGS. 18C-18D), the presence of fibrin was minimal in low-uptake specimens (FIGS. 18I-18L). Scale bar: 100 μm in FIGS. 18C, 18E, 18I, 18K; 200 μm in FIGS. 18D, 18F, 18J, 18L. Autoradiography (FIG. 18M) and functional probe assay (FIG. 18N) of discarded endarterectomy specimens from all 12 patients revealed a variable but significantly increased (P<0.05) uptake of fibrin-binding probe ⁶⁸Ga-20 compared to non-binding probe ⁶⁸Ga-22. Dashed lines: ⁶⁸Ga-22 Mean, Mean±SD, and Mean±2SD cutoff lines.

### DETAILED DESCRIPTION

Thromboembolism plays a causative role in a number of potentially mortal cardiovascular events including stroke, coronary events, deep vein thrombosis, and pulmonary embolism. Of the nearly 795,000 strokes that occur in the U.S. each year, more than 80% of are ischemic, or thromboembolic, in nature. Treatment options are largely influenced by the anatomical location of the thrombus. Currently, multiple tests are required to assess each bodily region, for example, CT scans are used to locate lung thrombi, ultrasound tests can be employed for carotid arteries, and MRI analysis provides images of the heart chambers. Rapid location of the culprit clot(s) is necessary to determine proper therapeutic recourse.

Many approaches have been developed for the visualization of thrombi throughout the body. Fibrin is a particularly attractive target as it is present in all thrombi, including arterial, venous, and cardiac; it is not found in plasma, rendering the visualization technique highly specific; it is accessible in all active stages of clot development; and it is a high concentration target at about 20-100 μM concentration. Fibrin-binding peptides can be functionalized, for example, with a chelating moiety that is able to chelate radioactive isotopes of various metals, including, but not limited to, actinium-225, bismuth-213, copper-64, copper-67, aluminum fluoride (Al¹⁸F), gallium-68, holmium-166, indium-111, lead-203, lead-212, lutetium-177, radium-223, samarium-153, scandium-43, scandium-44, scandium-47, terbium-149, terbium-152, terbium-155, terbium-161, thorium-227, yttrium-86, yttrium-90, and zirconium-89. Fibrin-binding peptides can also be functionalized with radioisotopes (including, but not limited to, ¹⁸F, ¹²³I, ¹²⁴I, ¹³¹I, and ²¹¹At) through direct covalent modification or indirect covalent modification through a linker, but which does not require a chelating group. In some embodiments, the radioisotopes (which includes radioactive isotopes of metals) are useful as imaging or diagnostic agents. In some embodiments, the radioisotopes (including radioactive isotopes of metals) are useful as therapeutic agents. Provided herein are fibrin-specific compounds comprising one or more radioisotopes. Also provided are methods for imaging fibrin. Also provided are methods for treating diseases or disorders using the fibrin-specific compounds of the present disclosure as imaging or diagnostic agents, therapeutic agents, or both.

### Definitions

Commonly used chemical abbreviations that are not explicitly defined in this disclosure may be found in The American Chemical Society Style Guide, Second Edition, American Chemical Society, Washington, D.C. (1997); “2001 Guidelines for Authors,” J. Org. Chem. 66(1), 24A (2001); and “A Short Guide to Abbreviations and Their Use in Peptide Science,” J. Peptide Sci. 5, 465-471 (1999).

As used herein, the term “peptide” refers to a chain of amino acids that is about 2 to about 25 amino acid residues in length. All peptide sequences herein are written from the N- to C-terminus. For any of the peptides described herein that contain two or more cysteine residues, it is understood that the cysteine residues can form one or more disulfide bonds under non-reducing conditions. Formation of a disulfide bond can result in the formulation of a cyclic peptide.

As used herein, the terms “natural” or “naturally occurring” amino acid refer to one of the twenty most common amino acids occurring in nature. Natural amino acids modified to provide a label for detection purposes (e.g., radioactive labels, optical labels, or dyes) are considered to be natural amino acids. Natural L amino acids are referred to by their standard one- or three-letter abbreviations. D amino acids are referred to using the lower-case convention for standard one-letter abbreviations, and the “D-” prefix convention for standard three-letter abbreviations.

As used herein, the terms “chelator,” “chelating group” and “chelating moiety” refer to a polydentate (multiple bonded) ligand that can form two or more separate coordinate bonds between the ligand and a single central atom, typically a metal ion. In some embodiments, the metal ion is a radioactive isotope of a metal. Examples of such metal ions include, but are not limited to, actinium-225, bismuth-213, copper-64, copper-67, gallium-68, holmium-166, indium-111, lead-203, lead-212, lutetium-177, radium-223, samarium-153, scandium-43, scandium-44, scandium-47, terbium-149, terbium-152, terbium-155, terbium-161, thorium-227, yttrium-86, yttrium-90, and zirconium-89.

As used herein, the terms “radioactive isotope,” “radioisotope,” “radionuclide,” and “radioactive nuclide” can be used interchangeably and refer to an unstable atom having excess nuclear energy; such excess energy can be emitted through one of three ways: emission from the nucleus as gamma radiation; transfer and release of one of its electrons as a conversion electron; or emission of a new particle (alpha or beta particle) from the nucleus. Such processes are known as radioactive decay of a radioisotope. Radioisotopes can be used for diagnostic imaging and for the treatment of a variety of diseases and conditions, including those described in the present disclosure.

As used herein, the terms “target binding” and “binding” refer to non-covalent interactions of a peptide or composition within a target. These non-covalent interactions are independent from one another and may be, inter alia, hydrophobic, hydrophilic, dipole-dipole, pi-stacking, hydrogen bonding, electrostatic associations, and/or Lewis acid-base interactions. The binding affinity for a target is expressed in terms of the equilibrium dissociation constant “K_{d}” to the target under a defined set of conditions.

As used herein, the term “purified” refers to a peptide or compound that has been separated from either naturally occurring organic molecules with which it normally associates or, for a chemically-synthesized molecule, separated from other organic molecules present in the chemical synthesis. Typically, the polypeptide or compound is considered “purified” when it is at least 70% (e.g., at least 70%, at least 80%, at least 90%, at least 95%, or at least 99%), by dry weight, free from any other proteins or organic molecules. The terms “purified” and isolated” are used interchangeably herein.

The terms “percent identity” or “identity” in the context of two or more nucleic acids or polypeptides, refer to two or more sequences that are the same or have a specified percentage of nucleotides or amino acid residues that are the same. The percent identity can be measured using sequence comparison software or algorithms or by visual inspection.

In general, percent sequence identity is calculated by determining the number of matched positions in aligned nucleic acid or polypeptide sequences, dividing the number of matched positions by the total number of aligned nucleotides or amino acids, respectively, and multiplying by 100. A matched position refers to a position in which identical nucleotides or amino acids occur at the same position in aligned sequences. The total number of aligned nucleotides or amino acids refers to the minimum number of nucleotides or amino acids that are necessary to align the second sequence, and does not include alignment (e.g., forced alignment) with non-fibrin binding sequences. The total number of aligned nucleotides or amino acids may correspond to the entire sequence or may correspond to fragments of the full-length sequence.

Sequences can be aligned using the algorithm described by Altschul et al. (Nucleic Acids Res, 25:3389-3402, 1997) as incorporated into BLAST (basic local alignment search tool) programs, available at ncbi.nlm.nih.gov on the World Wide Web. BLAST searches or alignments can be performed to determine percent sequence identity between a nucleic acid or polypeptide and any other sequence or portion thereof using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a fibrin-binding sequence and another sequence, the default parameters of the respective programs are used.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### Compounds

Provided herein are compounds of Formula I:

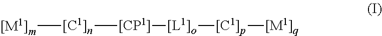

or a pharmaceutically acceptable salt thereof,
   wherein each M¹ is independently copper-64 or gallium-68;
   each C¹ is a chelating moiety independently selected from the group consisting of:

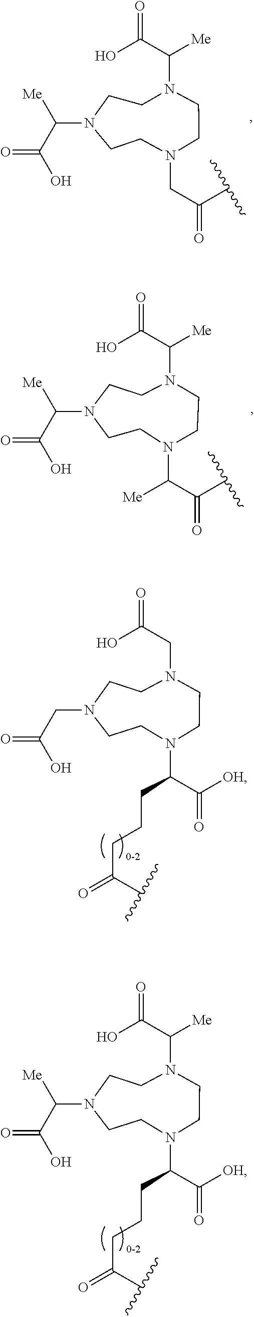

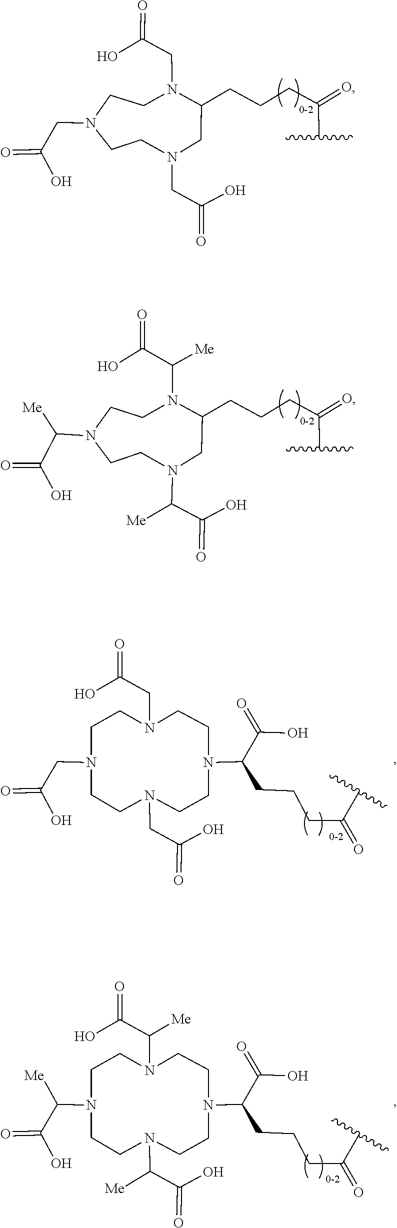

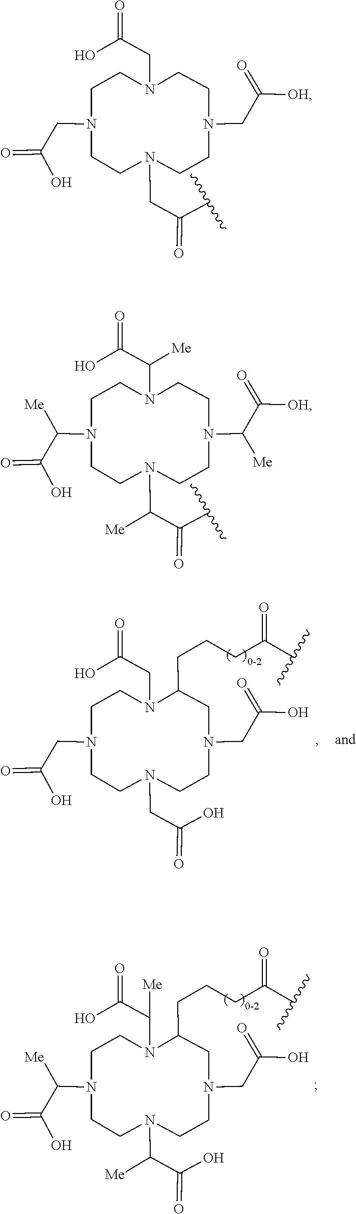

CP¹ is a fibrin-binding peptide;
each L¹ is independently a linker moiety;
m is an integer selected from 0 to 5;
n is an integer selected from 0 to 5;
is an integer selected from 0 to 5;
p is an integer selected from 0 to 5; and
q is an integer selected from 0 to 5.

In some embodiments of Formula I, each M¹ is copper-64. In some embodiments of Formula I, each M¹ is gallium-68.

In some embodiments of Formula I, each C¹ is independently selected from the group consisting of:

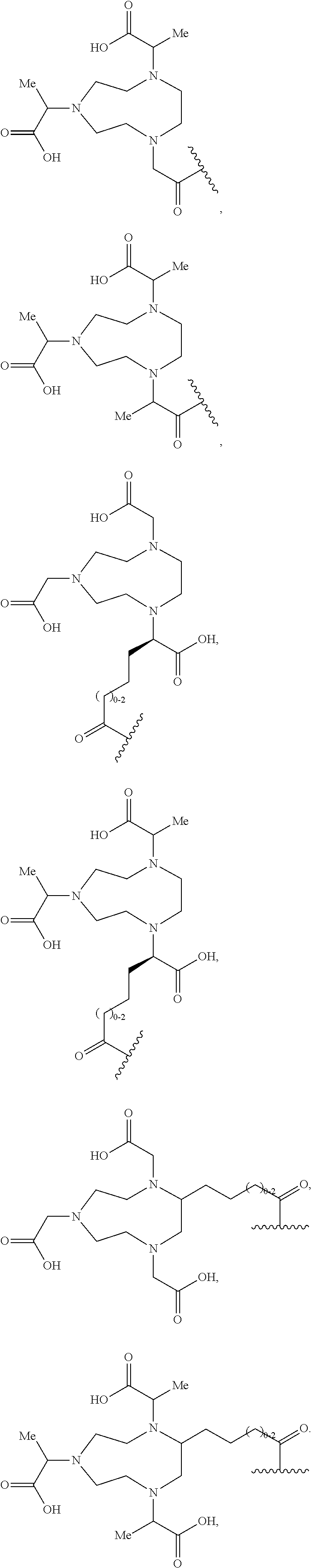

In some embodiments of Formula I, C¹ is NODAGA:

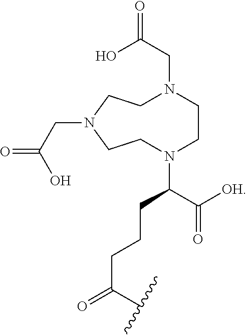 [SMILES: *C(=O)CCC[C@H](C(=O)O)N1CCN(CC(=O)O)CCN(CC(=O)O)CC1]

In some embodiments of Formula I, each C¹ is independently selected from the group consisting of:

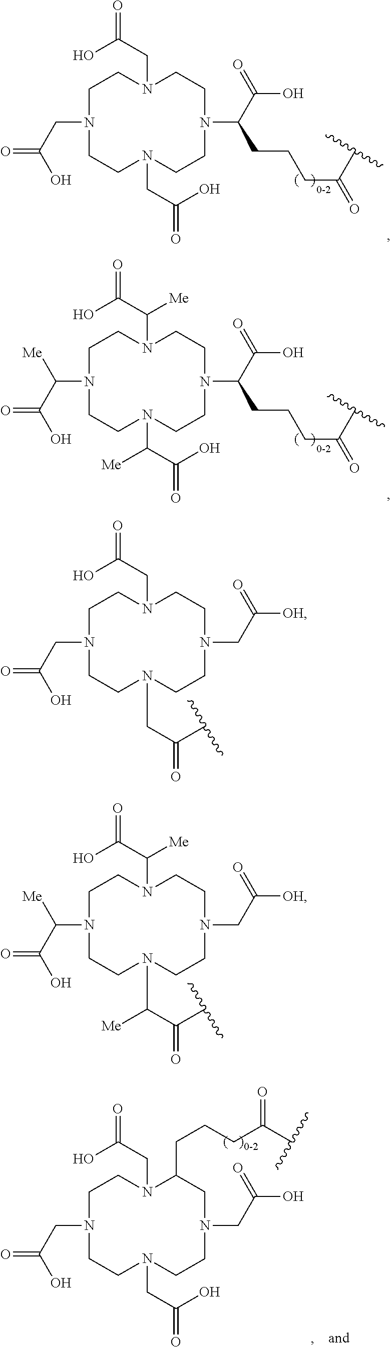

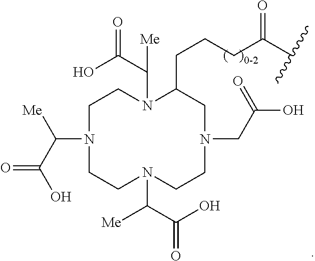

In some embodiments of Formula I, CP¹ is a fibrin-binding peptide comprising a sequence having at least 80% sequence identity to the polypeptide of SEQ ID NO: 1:
y*-X¹—C-Hyp-Y(3-Cl)—X²-L-C—X³—I—X⁴— (SEQ ID NO:1)
   wherein each of X¹, X², X³, and X⁴ is independently any amino acid; and y* is L-tyrosine or D-tyrosine. For example, CP¹ is a fibrin-binding peptide comprising a sequence having at least 85%, at least 90%, at least 92% at least 95%, at least 98%, at least 99%, or 100% sequence identity to the polypeptide of SEQ ID NO:1. In some embodiments, CP¹ is the polypeptide of SEQ ID NO:1 (i.e., it has 100% sequence identity).

In some embodiments, y* is L-tyrosine. In some embodiments, y* is D-tyrosine.

In some embodiments, each of X¹, X², X³, and X⁴ is independently selected from naturally occurring amino acids. For example, each of X¹, X², X³, and X⁴ is independently selected from Ala, Cys, Asp, Glu, Phe, Gly, His, Ile, Lys, Leu, Met, Asn, Pro, Gln, Arg, Ser, Thr, Val, Trp, and Tyr. In some embodiments, each of X¹, X², X³, and X⁴ is independently selected from the D-configuration of the naturally occurring amino acids. For example, each of X¹, X², X³, and X⁴ is independently selected from D-Ala, D-Cys, D-Asp, D-Glu, D-Phe, D-His, D-Ile, D-Lys, D-Leu, D-Met, D-Asn, D-Pro, D-Gln, D-Arg, D-Ser, D-Thr, D-Val, D-Trp, and D-Tyr. In some embodiments, X¹ is Glu. In some embodiments, X¹ is D-His. In some embodiments, X² is Gly. In some embodiments, X² is Asp. In some embodiments, X² is D-Asp. In some embodiments, X³ is His. In some embodiments, X³ is Tyr. In some embodiments, X⁴ is Gin. In some embodiments, X⁴ is D-Gin. In some embodiments, X⁴ is Leu. In some embodiments, X⁴ is D-Leu.

In some embodiments, each of X¹, X², X³, and X⁴ is independently selected from non-naturally occurring amino acids. For example, each of X¹, X², X³, and X⁴ is independently selected from Hyp, D-Hyp, Tyr-3-Cl, and D-Tyr-3-Cl.

In some embodiments of Formula I, CP¹ is a fibrin-binding peptide comprising a polypeptide having at least 80% (e.g., at least 85%, at least 90%, at least 92%, at least 95%, at least 98%, at least 99% or 100%) sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments of Formula I, CP¹ is a fibrin-binding peptide having at least 80% (e.g., at least 85%, at least 90%, at least 92%, at least 95%, at least 98%, at least 99%, or 100%) sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments of Formula I, each L¹ is independently selected from the group consisting of:

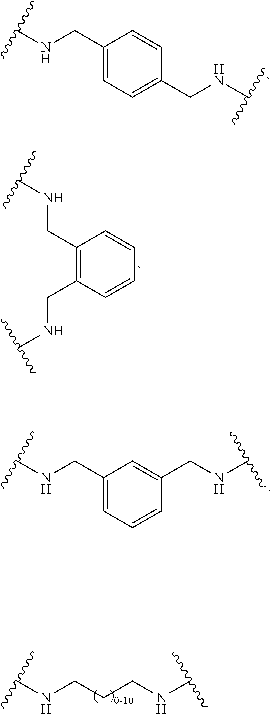

In some embodiments, m is 1. In some embodiments, m is 2. In some embodiments, p is 1. In some embodiments, p is 2. In some embodiments, n is 1. In some embodiments, n is 2. In some embodiments, o is 1. In some embodiments, o is 2. In some embodiments, q is 1. In some embodiments, q is 2. In some embodiments, each of m, p, n, o, and q is 1. In some embodiments, each of m, p, n, o, and q is 2.

In some embodiments of Formula I, the compound is selected from the group consisting of:

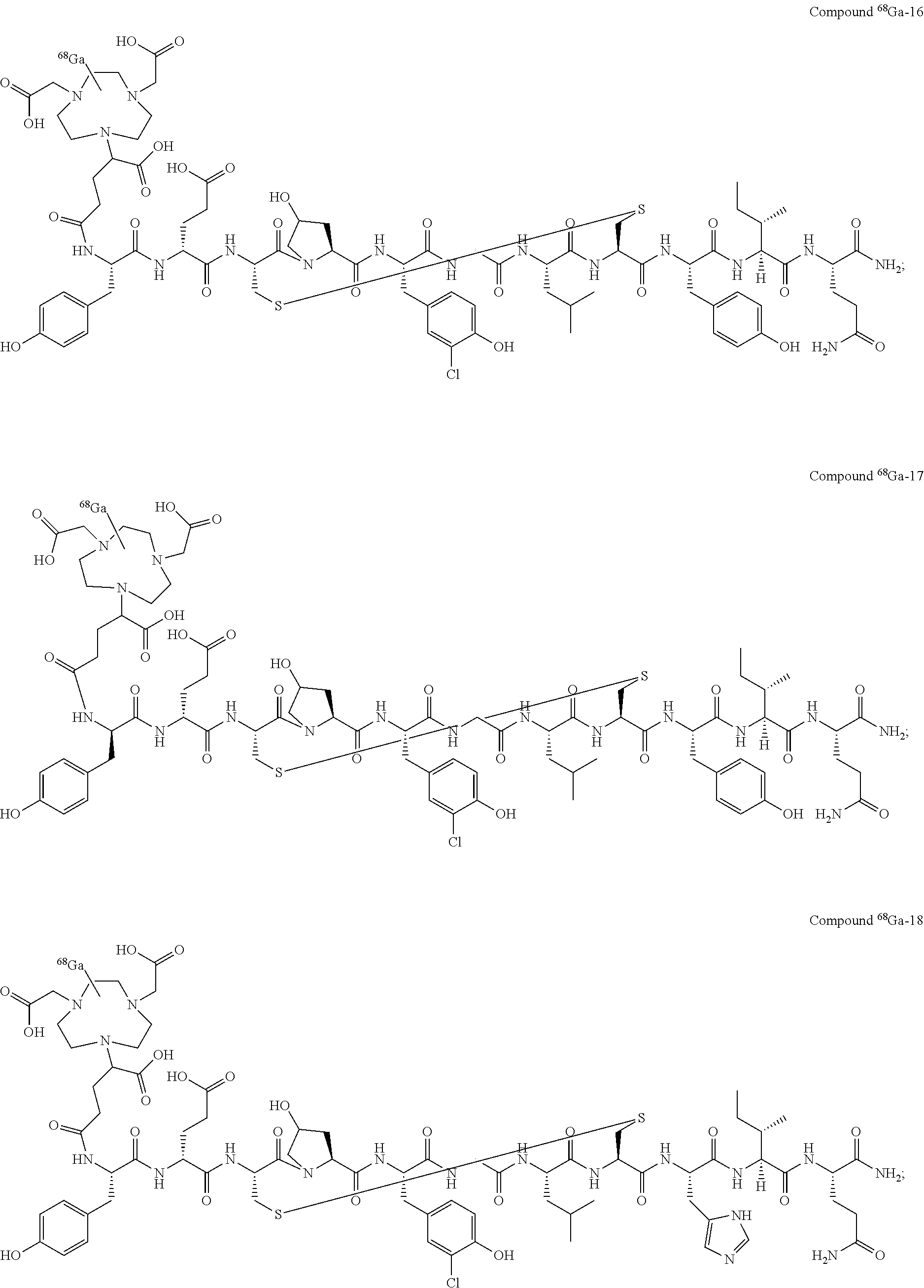

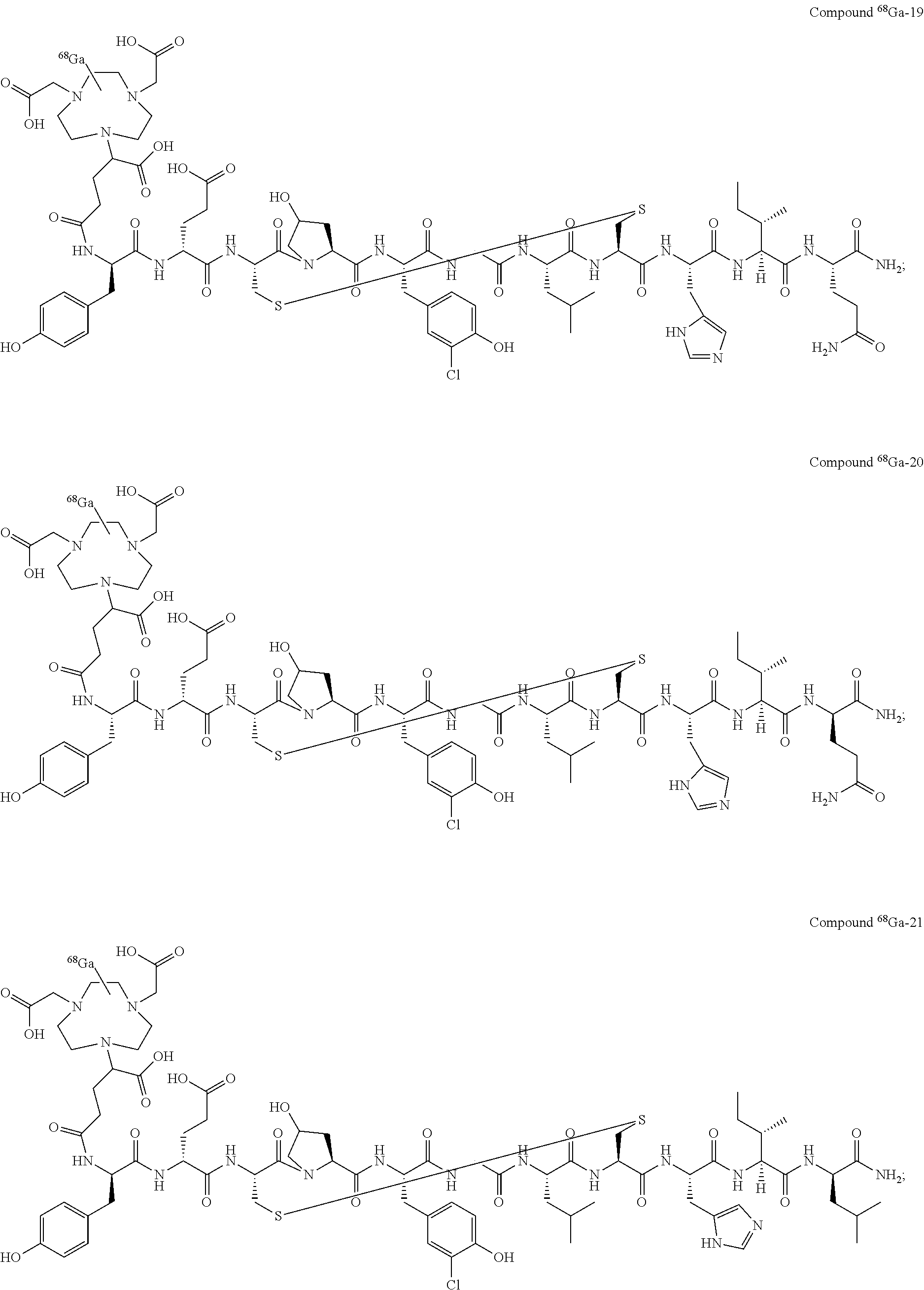

or a pharmaceutically acceptable salt thereof.

In some embodiments of Formula I, the compound is selected from the group consisting of:

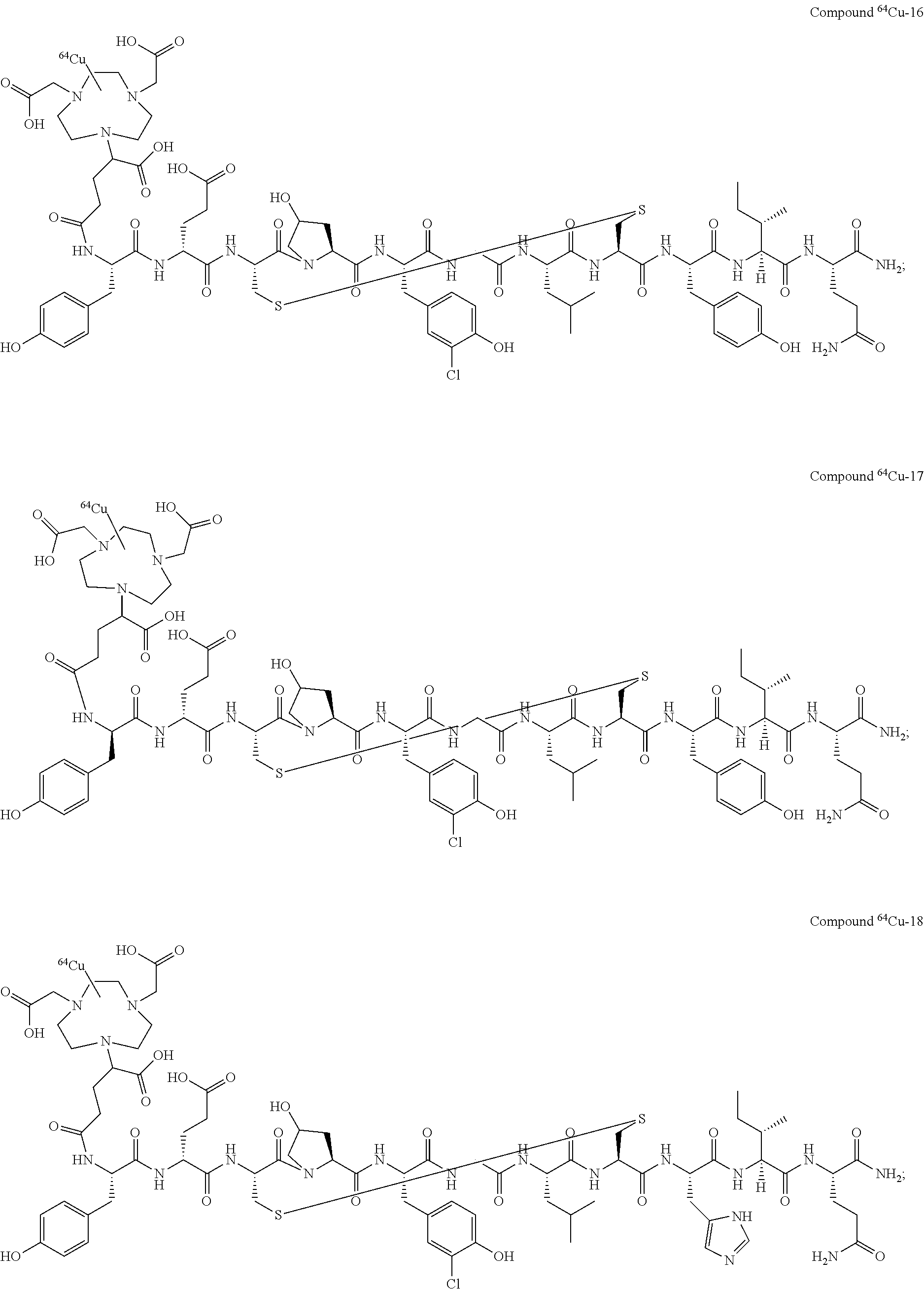

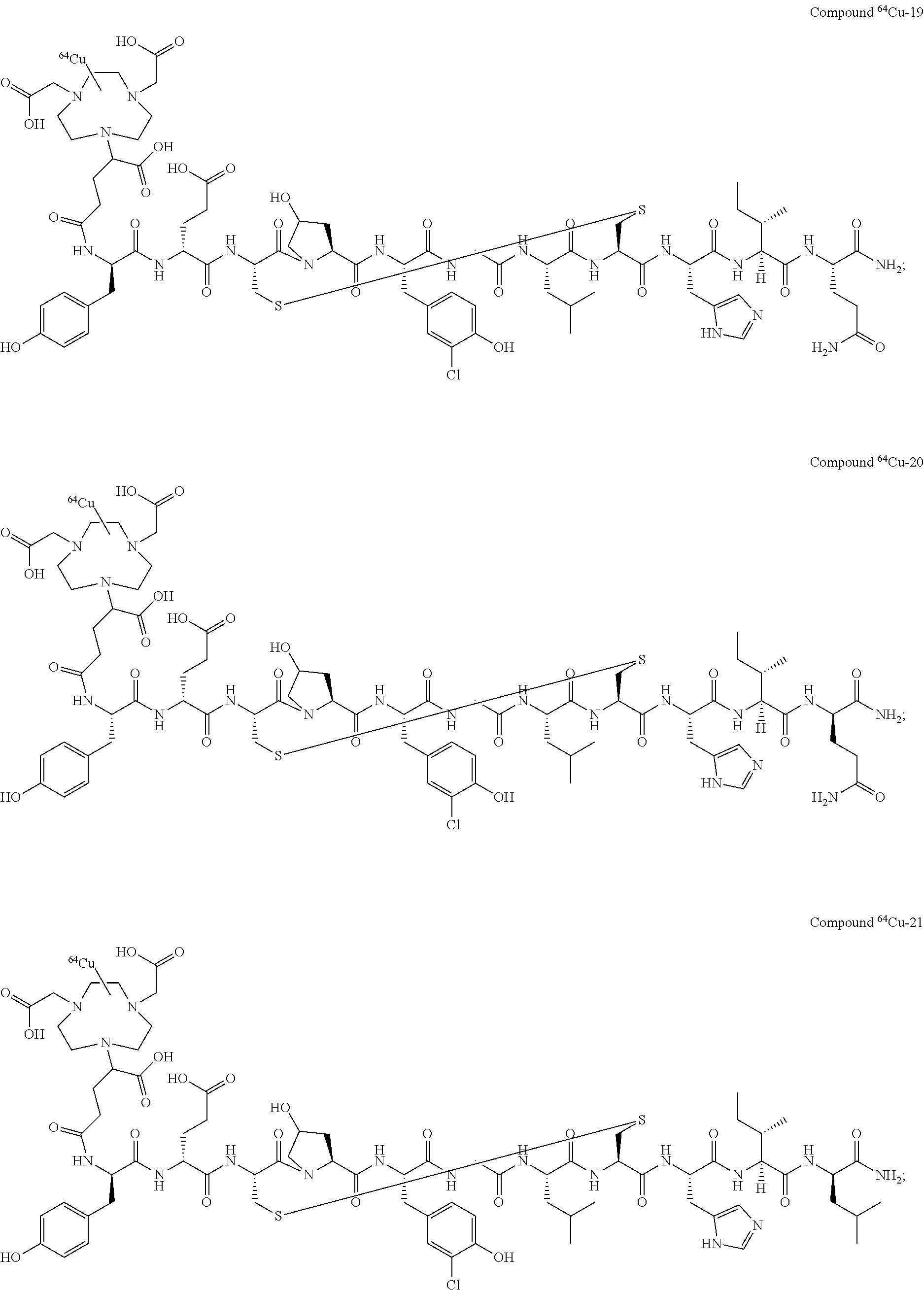

or a pharmaceutically acceptable salt thereof.

Also provided herein are compounds of Formula Ia:

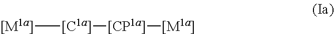

or a pharmaceutically acceptable salt thereof,
   wherein each M^{1a} is independently copper-64 or gallium-68;
   C^{1a} is a chelating moiety independently selected from the group consisting of:

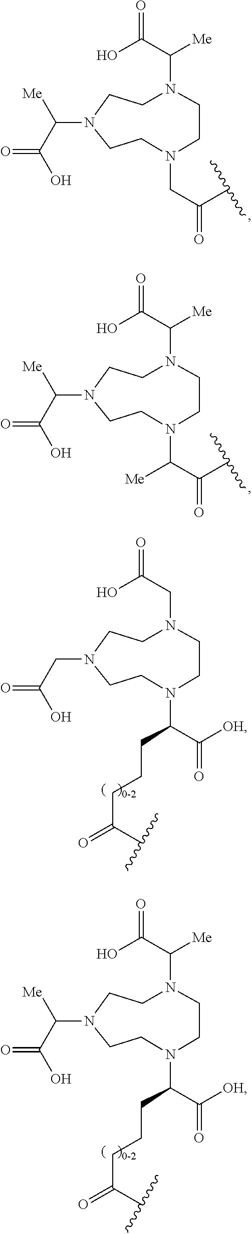

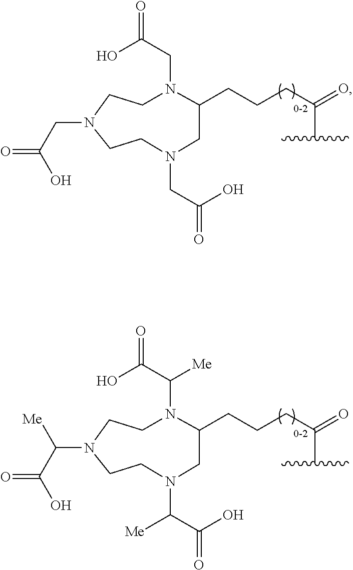

and CP^{1a} is a fibrin-binding peptide.

In some embodiments of Formula Ia, each M^{1a} is copper-64. In some embodiments of Formula Ia, each M^{1a} is gallium-68.

In some embodiments of Formula Ia, C^{1a} is NODAGA:

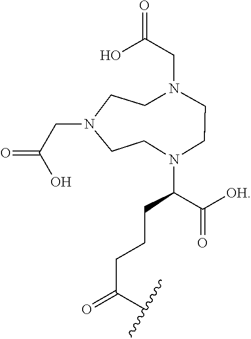 [SMILES: *C(=O)CCC[C@H](C(=O)O)N1CCN(CC(=O)O)CCN(CC(=O)O)CC1]

In some embodiments of Formula Ia, CP^{1a} is a fibrin-binding peptide comprising a sequence having at least 80% sequence identity to the polypeptide of SEQ ID NO:1:

(SEQ ID NO: 1)
-y*-X¹-C-Hyp-Y(3-Cl)-X²-L-C-X³-I-X⁴-

wherein each of X¹, X², X³, and X⁴ is independently any amino acid; and y* is L-tyrosine or D-tyrosine. For example, CP^{1a} is a fibrin-binding peptide comprising a sequence having at least 85%, at least 90%, at least 92% at least 95%, at least 98%, at least 99%, or 100% sequence identity to the polypeptide of SEQ ID NO:1. In some embodiments, CP^{1a} is the polypeptide of SEQ ID NO:1 (i.e., it has 100% sequence identity).

In some embodiments, y* is L-tyrosine. In some embodiments, y* is D-tyrosine.

In some embodiments, each of X¹, X², X³, and X⁴ is independently selected from naturally occurring amino acids. For example, each of X¹, X², X³, and X⁴ is independently selected from Ala, Cys, Asp, Glu, Phe, Gly, His, Ile, Lys, Leu, Met, Asn, Pro, Gln, Arg, Ser, Thr, Val, Trp, and Tyr. In some embodiments, each of X¹, X², X³, and X⁴ is independently selected from the D-configuration of the naturally occurring amino acids. For example, each of X¹, X², X³, and X⁴ is independently selected from D-Ala, D-Cys, D-Asp, D-Glu, D-Phe, D-His, D-Ile, D-Lys, D-Leu, D-Met, D-Asn, D-Pro, D-Gln, D-Arg, D-Ser, D-Thr, D-Val, D-Trp, and D-Tyr. In some embodiments, X¹ is Glu. In some embodiments, X¹ is D-His. In some embodiments, X² is Gly. In some embodiments, X² is Asp. In some embodiments, X² is D-Asp. In some embodiments, X³ is His. In some embodiments, X³ is Tyr. In some embodiments, X⁴ is Gln. In some embodiments, X⁴ is D-Gln. In some embodiments, X⁴ is Leu. In some embodiments, X⁴ is D-Leu.

In some embodiments, each of X¹, X², X³, and X⁴ is independently selected from non-naturally occurring amino acids. For example, each of X¹, X², X³, and X⁴ is independently selected from Hyp, D-Hyp, Tyr-3-Cl, and D-Tyr-3-Cl.

In some embodiments of Formula Ia, CP^{1a} is a fibrin-binding peptide comprising a polypeptide having at least 80% (e.g., at least 85%, at least 90%, at least 92%, at least 95%, at least 98%, at least 99%, or 100%) sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments of Formula Ia, CP^{1a} is a fibrin-binding peptide having at least 80% (e.g., at least 85%, at least 90%, at least 92%, at least 95%, at least 98%, at least 99%, or 100%) sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-c-C-Hyp-Y(3-Cl)-d-L-C-Y-I-Q- |
| 15 | -Y-h-C-Hyp-Y(3-Cl)-G-L-C-Y-I-Q- |

Also provided herein are compounds of Formula Ib:

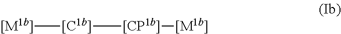

or a pharmaceutically acceptable salt thereof,
   wherein each M^{1b} is independently copper-64 or gallium-68;
   C^{1b} is NODAGA:

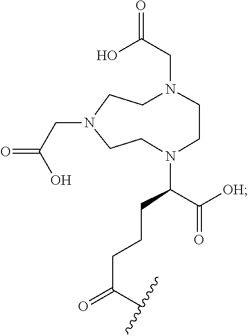 [SMILES: *C(=O)CCC[C@H](C(=O)O)N1CCN(CC(=O)O)CCN(CC(=O)O)CC1]

and
   CP^{1b} is a fibrin-binding peptide.

In some embodiments of Formula Ib, each M^{1b} is copper-64. In some embodiments of Formula Ib, each M^{1b} is gallium-68.

In some embodiments of Formula Ib, CP^{1b} is a fibrin-binding peptide comprising a sequence having at least 80% sequence identity to the polypeptide of SEQ ID NO:1:

(SEQ ID NO: 1)
-y*-X¹-C-Hyp-Y(3-Cl)-X²-L-C-X³-I-X⁴-

wherein each of X¹, X², X³, and X⁴ is independently any amino acid; and y* is L-tyrosine or D-tyrosine. For example, CP^{1b} is a fibrin-binding peptide comprising a sequence having at least 85%, at least 90%, at least 92% at least 95%, at least 98%, at least 99%, or 100% sequence identity to the polypeptide of SEQ ID NO:1. In some embodiments, CP^{1b} is the polypeptide of SEQ ID NO:1 (i.e., it has 100% sequence identity).

In some embodiments, y* is L-tyrosine. In some embodiments, y* is D-tyrosine.

In some embodiments, each of X¹, X², X³, and X⁴ is independently selected from naturally occurring amino acids. For example, each of X¹, X², X³, and X⁴ is independently selected from Ala, Cys, Asp, Glu, Phe, Gly, His, Ile, Lys, Leu, Met, Asn, Pro, Gln, Arg, Ser, Thr, Val, Trp, and Tyr. In some embodiments, each of X¹, X², X³, and X⁴ is independently selected from the D-configuration of the naturally occurring amino acids. For example, each of X¹, X², X³, and X⁴ is independently selected from D-Ala, D-Cys, D-Asp, D-Glu, D-Phe, D-His, D-Ile, D-Lys, D-Leu, D-Met, D-Asn, D-Pro, D-Gln, D-Arg, D-Ser, D-Thr, D-Val, D-Trp, and D-Tyr. In some embodiments, X¹ is Glu. In some embodiments, X¹ is D-His. In some embodiments, X² is Gly. In some embodiments, X² is Asp. In some embodiments, X² is D-Asp. In some embodiments, X³ is His. In some embodiments, X³ is Tyr. In some embodiments, X⁴ is Gin. In some embodiments, X⁴ is D-Gin. In some embodiments, X⁴ is Leu. In some embodiments, X⁴ is D-Leu.

In some embodiments, each of X¹, X², X³, and X⁴ is independently selected from non-naturally occurring amino acids. For example, each of X¹, X², X³, and X⁴ is independently selected from Hyp, D-Hyp, Tyr-3-Cl, and D-Tyr-3-Cl.

In some embodiments of Formula Ib, Cp^{1b} is a fibrin-binding peptide comprising a polypeptide having at least 80% (e.g., at least 85%, at least 90%, at least 92%, at least 95%, at least 98%, at least 99%, or 100%) sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments of Formula Ib, CP^{1b} is a fibrin-binding peptide having at least 80% (e.g., at least 85%, at least 90%, at least 92%, at least 95%, at least 98%, at least 99%, or 100%) sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

Also provided herein are compounds of Formula II:

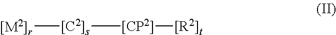

or a pharmaceutically acceptable salt thereof,
   wherein each M² is independently copper-64 or gallium-68;
   each C² is independently a chelating moiety;
   CP² is a fibrin-binding peptide;
   each R² is independently an organic, non-chelating moiety;
   r is an integer selected from 0 to 5;
   s in an integer selected from 0 to 5; and
   t is an integer selected from 0 to 5.

In some embodiments of Formula II, each M² is copper-64. In some embodiments of Formula II, each M² is gallium-68.

In some embodiments of Formula II, each C² is independently selected from the group consisting of:

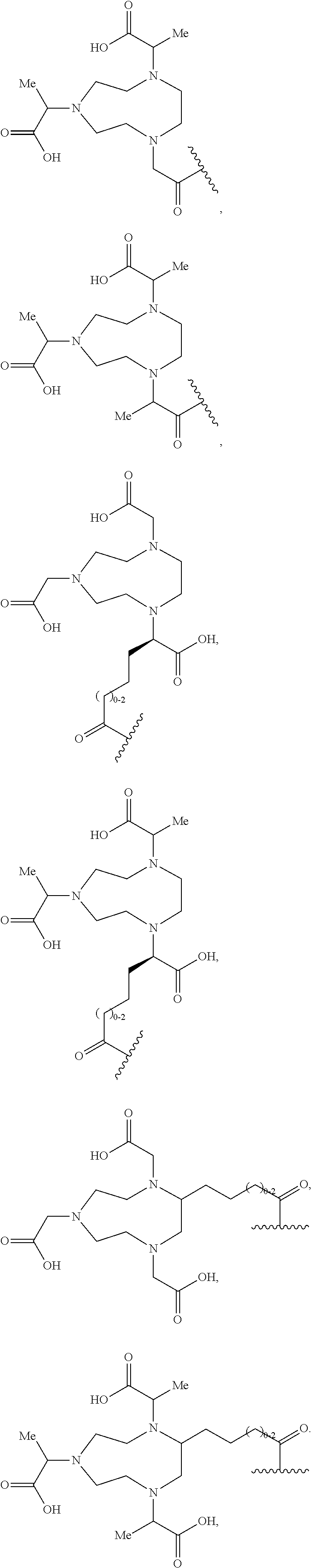

In some embodiments of Formula II, C² is NODAGA:

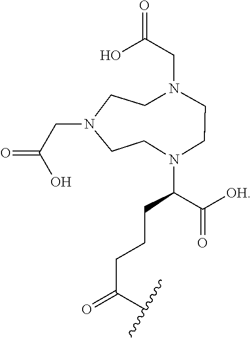 [SMILES: *C(=O)CCC[C@H](C(=O)O)N1CCN(CC(=O)O)CCN(CC(=O)O)CC1]

In some embodiments of Formula II, each C² is independently selected from the group consisting of:

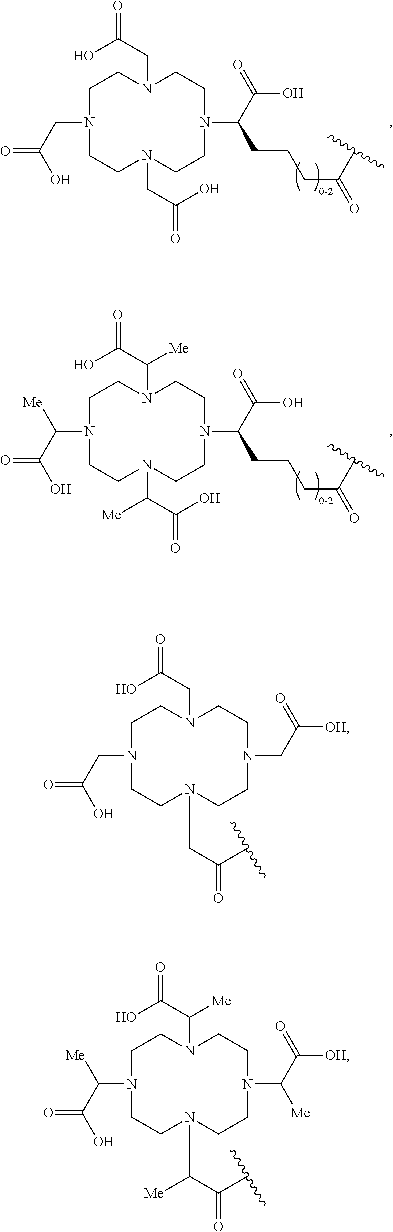

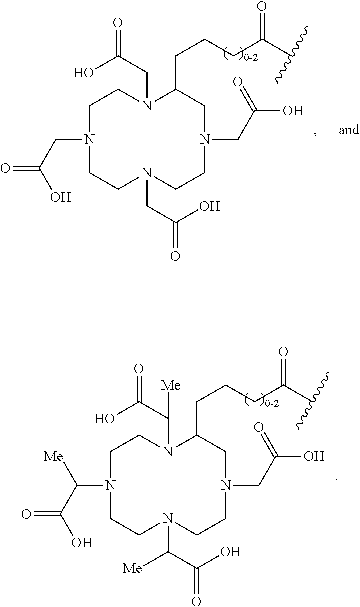

In some embodiments of Formula II, CP² is a fibrin-binding peptide comprising a sequence having at least 80% sequence identity to the polypeptide of SEQ ID NO:16:

(SEQ ID NO: 16)
-y*-X⁵-C-Hyp-Y(3-Cl)-X⁶-L-C-X⁷-I-X⁸-

wherein each of X⁵, X⁶, X⁷, and X⁸ is independently any amino acid; and y* is L-tyrosine or D-tyrosine. For example, CP² is a fibrin-binding peptide comprising a sequence having at least 85%, at least 90%, at least 92% at least 95%, at least 98%, at least 99%, or 100% sequence identity to the polypeptide of SEQ ID NO:16. In some embodiments, CP² is a fibrin-binding peptide of SEQ ID NO:16 (i.e., having 100% sequence identity to SEQ ID NO:16).

In some embodiments, y* is L-tyrosine. In some embodiments, y* is D-tyrosine.

In some embodiments, each of X⁵, X⁶, X⁷, and X⁸ is independently selected from naturally occurring amino acids. For example, each of X⁵, X⁶, X⁷, and X⁸ is independently selected from Ala, Cys, Asp, Glu, Phe, Gly, His, Ile, Lys, Leu, Met, Asn, Pro, Gln, Arg, Ser, Thr, Val, Trp, and Tyr. In some embodiments, each of X⁵, X⁶, X⁷, and X⁸ is independently selected from the D-configuration of the naturally occurring amino acids.

For example, each of X⁵, X⁶, X⁷, and X⁸ is independently selected from D-Ala, D-Cys, D-Asp, D-Glu, D-Phe, D-His, D-Ile, D-Lys, D-Leu, D-Met, D-Asn, D-Pro, D-Gln, D-Arg, D-Ser, D-Thr, D-Val, D-Trp, and D-Tyr. In some embodiments, X⁵ is Glu. In some embodiments, X⁵ is D-His. In some embodiments, X⁶ is Gly. In some embodiments, X⁶ is Asp. In some embodiments, X⁶ is D-Asp. In some embodiments, X⁷ is His. In some embodiments, X⁷ is Tyr. In some embodiments, X⁸ is Gln. In some embodiments, X⁸ is D-Gln. In some embodiments, X⁸ is Leu. In some embodiments, X⁸ is D-Leu.

In some embodiments, each of X⁵, X⁶, X⁷, and X⁸ is independently selected from non-naturally occurring amino acids. For example, each of X⁵, X⁶, X⁷, and X⁸ is independently selected from Hyp, D-Hyp, Tyr-3-Cl, and D-Tyr-3-Cl.

In some embodiments of Formula II, CP² is a fibrin-binding peptide comprising a polypeptide having at least 80% (e.g., at least 85%, at least 90%, at least 92%, at least 95%, at least 98%, at least 99%, or 100%) sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments of Formula II, CP² is a fibrin-binding peptide having at least 80% (e.g., at least 85%, at least 90%, at least 92%, at least 95% at least 98%, at least 99% or 100%) sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments of Formula II, each R² is independently selected from the group consisting of —N[(CH₂)ₐOR^{a}]₂, —NH(CH₂)ₐOR^{a}, —NH(CH₂)ₐOH, —NH(CH₂)CH₃, —N[(CH₂)ₐCH₃], —NH(CF₂)ₐCF₃,

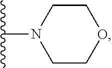 [SMILES: *N1CCOCC1]

### OR^{a}, and

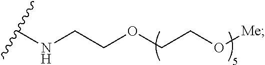

wherein each R^{a} is independently (C₁-C₆)alkyl; and
a is an integer selected from 0 to 5.

In some embodiments, r is 1. In some embodiments, r is 2. In some embodiments, s is 1. In some embodiments, s is 2. In some embodiments, t is 1. In some embodiments, t is 2. In some embodiments, each of r, s, and t is 1. In some embodiments, each of r, s, and tis 2.

Also provided herein are compounds of Formula III:

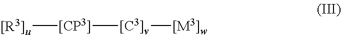

or a pharmaceutically acceptable salt thereof,
   wherein each M³ is independently copper-64 or gallium-68;
   each C³ is a chelating moiety;
   CP³ is a fibrin-binding peptide;
   each R³ is independently an organic, non-chelating moiety;
   u is an integer selected from 0 to 5;
   v in an integer selected from 0 to 5; and
   w is an integer selected from 0 to 5.

In some embodiments of Formula III, each M³ is copper-64. In some embodiments of Formula III, each M³ is gallium-68.

In some embodiments of Formula III, each C³ is independently selected from the group consisting of:

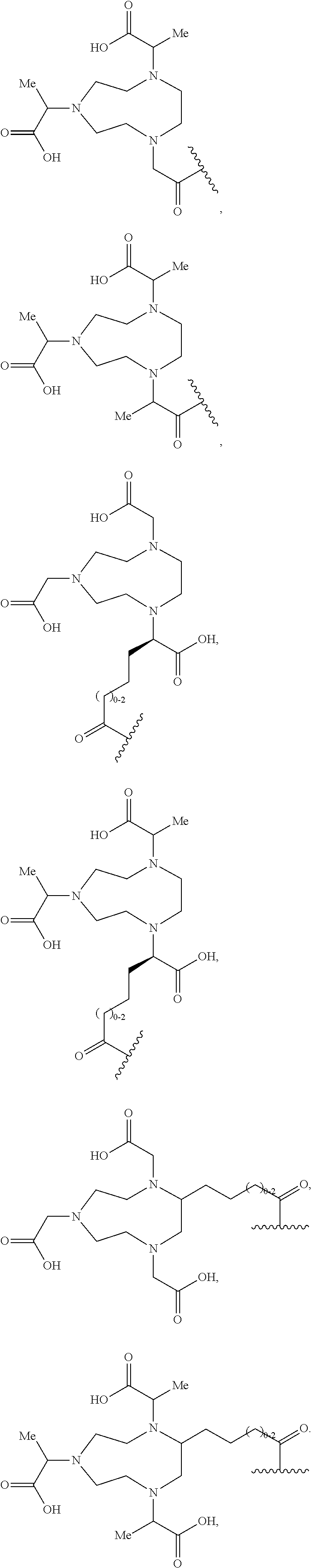

In some embodiments of Formula III, C³ is NODAGA:

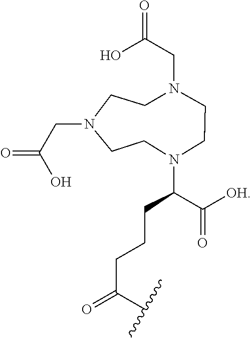 [SMILES: *C(=O)CCC[C@H](C(=O)O)N1CCN(CC(=O)O)CCN(CC(=O)O)CC1]

In some embodiments of Formula III, each C³ is independently selected from the group consisting of:

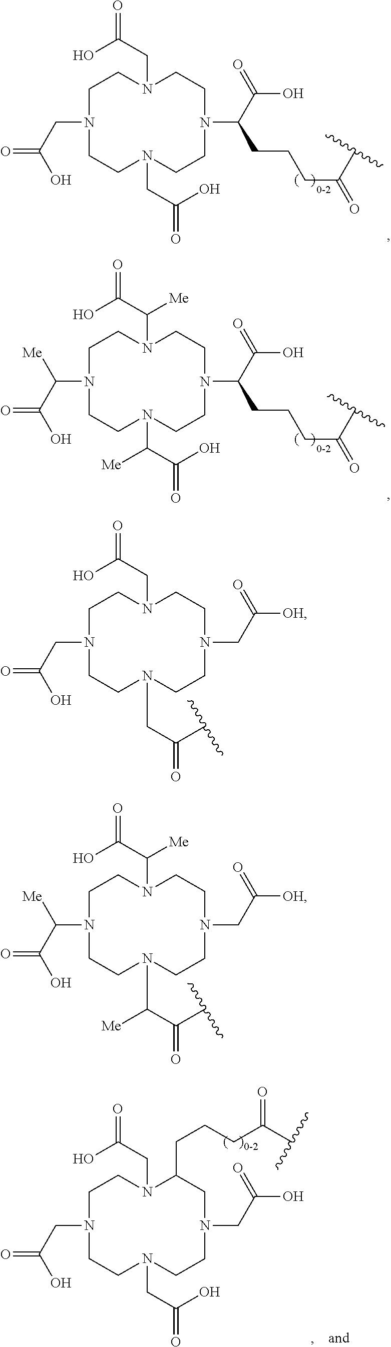

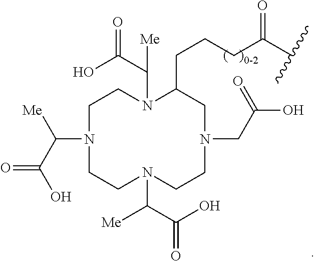

In some embodiments of Formula III, CP³ is a fibrin-binding peptide comprising a sequence having at least 80% sequence identity to the polypeptide of SEQ ID NO:17:

(SEQ ID NO: 17)
-y*-X⁹-C-Hyp-Y(3-Cl)-X¹⁰-L-C-X¹¹-I-X¹²-

wherein each of X⁹, X¹⁰, X¹¹, and X¹² is independently any amino acid; and y* is L tyrosine or D-tyrosine. For example, CP³ is a fibrin-binding peptide comprising a sequence having at least 85%, at least 90%, at least 92% at least 95%, at least 98%, at least 99%, or 100% sequence identity to the polypeptide of SEQ ID NO:17. In some embodiments, CP³ is a fibrin-binding peptide of SEQ ID NO:17 (i.e., having 100% sequence identity to SEQ ID NO:17).

In some embodiments, y* is L-tyrosine. In some embodiments, y* is D-tyrosine.

In some embodiments, each of X⁹, X¹⁰, X¹¹, and X¹² is independently selected from naturally occurring amino acids. For example, each of X⁹, X¹⁰, X¹¹, and X¹² is independently selected from Ala, Cys, Asp, Glu, Phe, Gly, His, Ile, Lys, Leu, Met, Asn, Pro, Gln, Arg, Ser, Thr, Val, Trp, and Tyr. In some embodiments, each of X⁹, X¹⁰, X¹¹, and X¹² is independently selected from the D-configuration of the naturally occurring amino acids. For example, each of X⁹, X¹⁰, X¹¹, and X¹² is independently selected from D-Ala, D-Cys, D-Asp, D-Glu, D-Phe, D-His, D-Ile, D-Lys, D-Leu, D-Met, D-Asn, D-Pro, D-Gln, D-Arg, D-Ser, D-Thr, D-Val, D-Trp, and D-Tyr. In some embodiments, X⁹ is Glu. In some embodiments, X⁹ is D-His. In some embodiments, X¹⁰ is Gly. In some embodiments, X¹⁰ is Asp. In some embodiments, X¹⁰ is D-Asp. In some embodiments, X¹¹ is His. In some embodiments, X¹¹ is Tyr. In some embodiments, X¹² is Gin. In some embodiments, X¹² is D-Gln. In some embodiments, X¹² is Leu. In some embodiments, X¹² is D-Leu.

In some embodiments, each of X⁹, X¹⁰, X¹¹, and X¹² is independently selected from non-naturally occurring amino acids. For example, each of X⁹, X¹⁰, X¹¹, and X¹² is independently selected from Hyp, D-Hyp, Tyr-3-Cl, and D-Tyr-3-Cl.

In some embodiments of Formula III, Cp³ is a fibrin-binding peptide comprising a polypeptide having at least 80% (e.g., at least 85%, at least 90%, at least 92%, at least 95%, at least 98%, at least 99%, or 100%) sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments of Formula III, Cp³ is a fibrin-binding peptide having at least 80% (e.g., at least 85%, at least 90%, at least 92%, at least 95% at least 98%, at least 99%, or 100%) sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments of Formula III, each R³ is independently selected from the group consisting of —N[(CH₂)_{b}OR^{b}]₂, —NH(CH₂)_{b}OR^{b}, —NH(CH₂)_{b}OH, —NH(CH₂)CH₃, —N[(CH₂)_{b}CH₃], —NH(CF₂)_{b}CF₃,

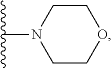 [SMILES: *N1CCOCC1]

### OR^{b}, and

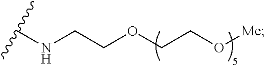

wherein each R^{b} is independently (C₁-C₆)alkyl; and
b is an integer selected from 0 to 5.

In some embodiments, u is 1. In some embodiments, u is 2. In some embodiments, v is 1. In some embodiments, v is 2. In some embodiments, w is 1. In some embodiments, w is 2. In some embodiments, each of u, v, and w is 1. In some embodiments, each of u, v, and wis 2.

Also provided are compounds of Formula IV:

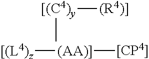

or a pharmaceutically acceptable salt thereof,
   wherein R⁴ is a radioisotope;
   C⁴ is a chelating moiety selected from the group consisting of:

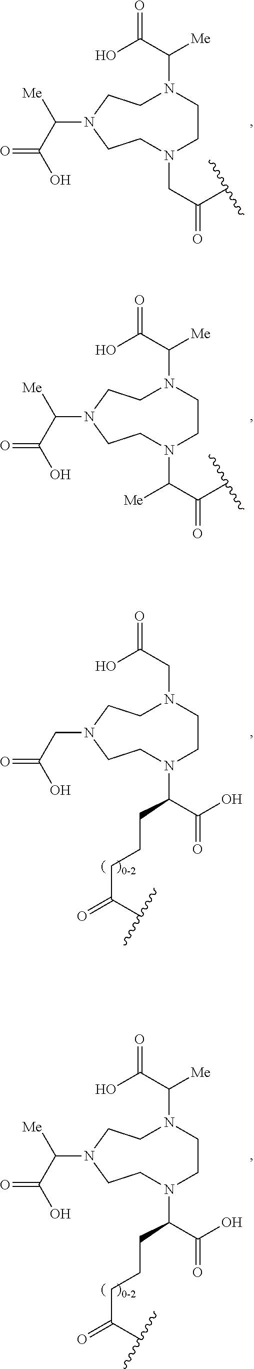

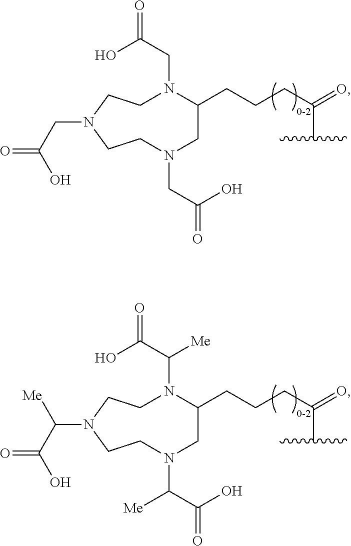

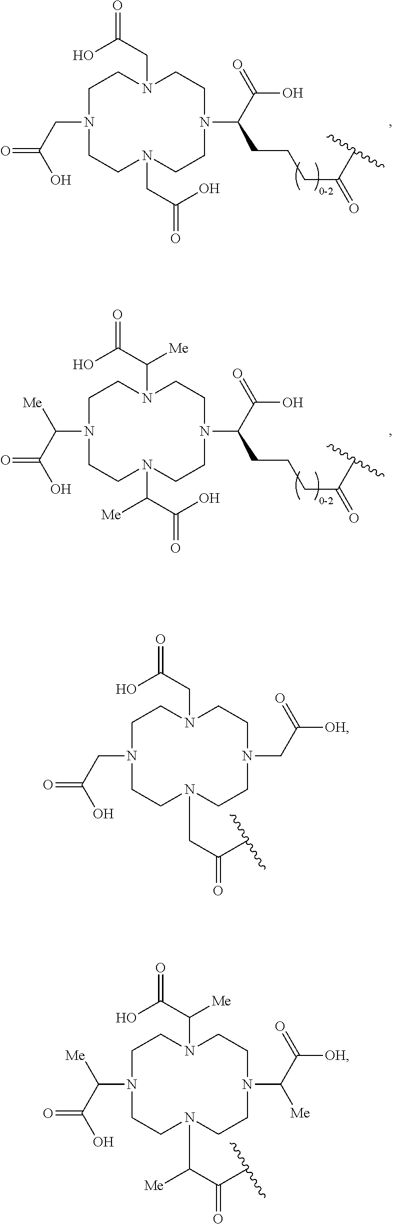

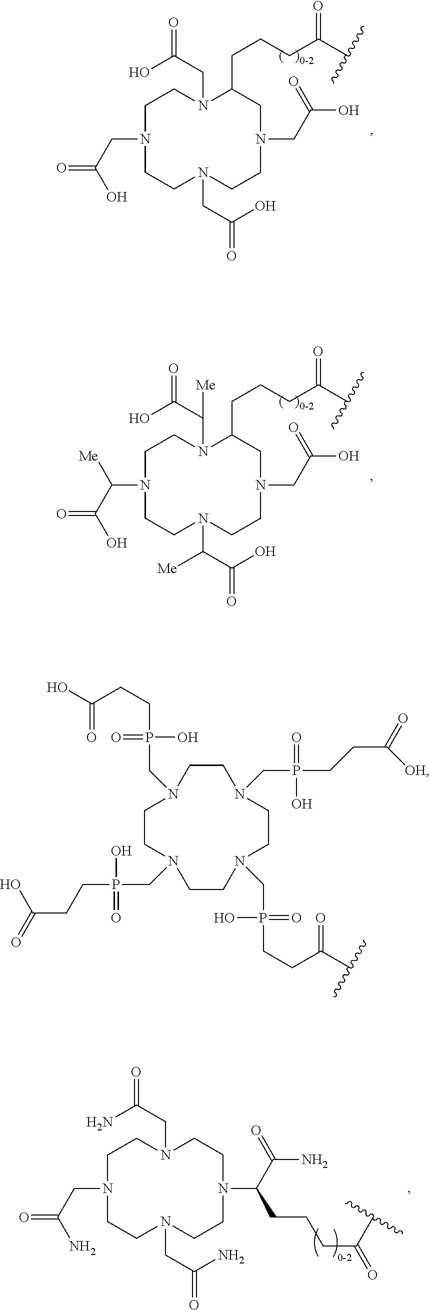

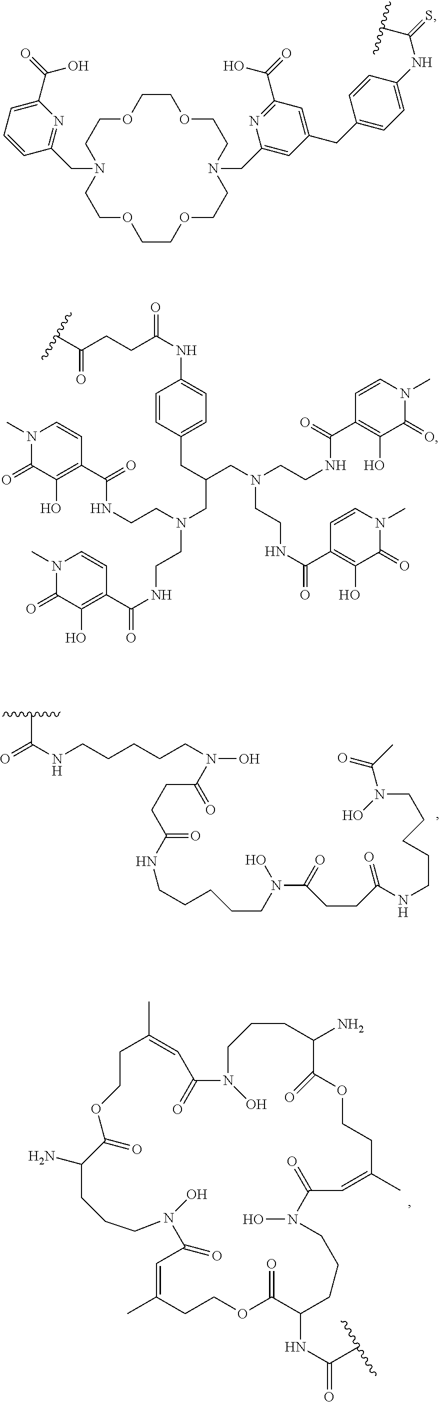 [SMILES: *C(=S)Nc1ccc(Cc2cc(CN3CCOCCOCCN(Cc4cccc(C(=O)O)n4)CCOCCOCC3)nc(C(=O)O)c2)cc1 ; *C(=O)CCC(=O)Nc1ccc(CC(CN(CCNC(=O)c2ccn(C)c(=O)c2O)CCNC(=O)c2ccn(C)c(=O)c2O)CN(CCNC(=O)c2ccn(C)c(=O)c2O)CCNC(=O)c2ccn(C)c(=O)c2O)cc1 ; *C(=O)NCCCCCN(O)C(=O)CCC(=O)NCCCCCN(O)C(=O)CCC(=O)NCCCCCN(O)C(C)=O ; *C(=O)NC1CCCN(O)C(=O)/C=C(/C)CCOC(=O)C(N)CCCN(O)C(=O)/C=C(/C)CCOC(=O)C(N)CCCN(O)C(=O)/C=C(/C)CCOC1=O]

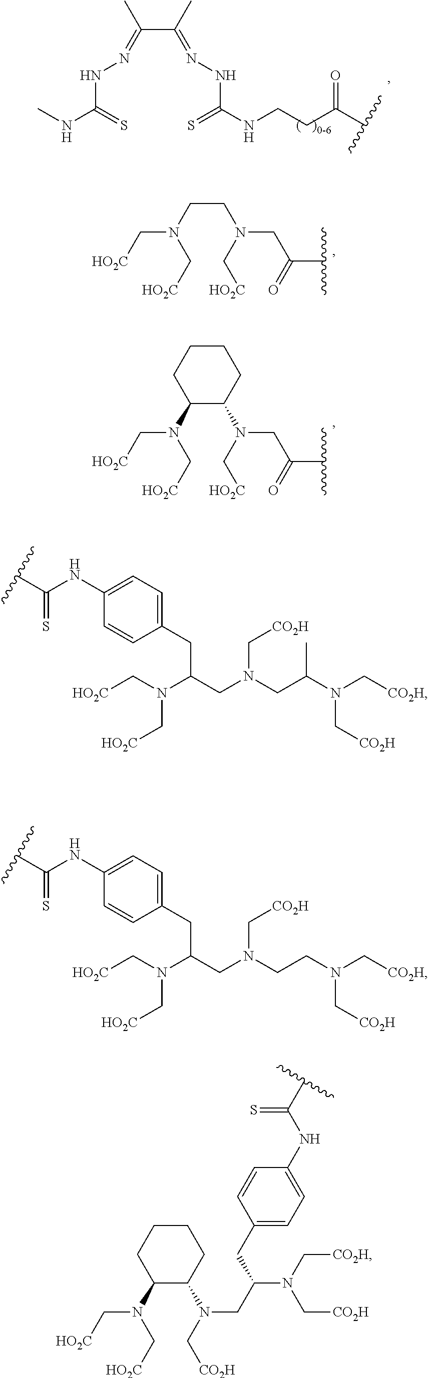

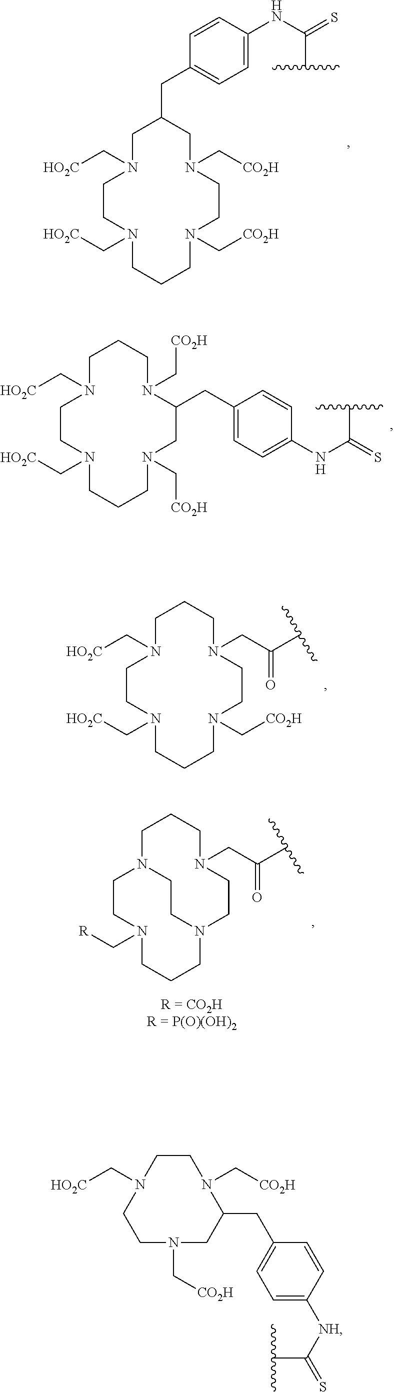

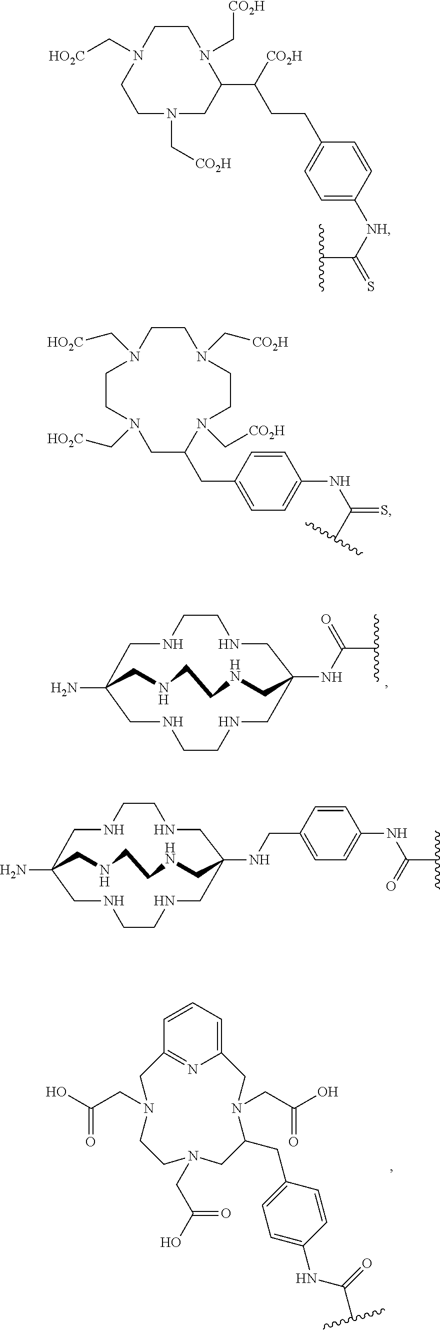

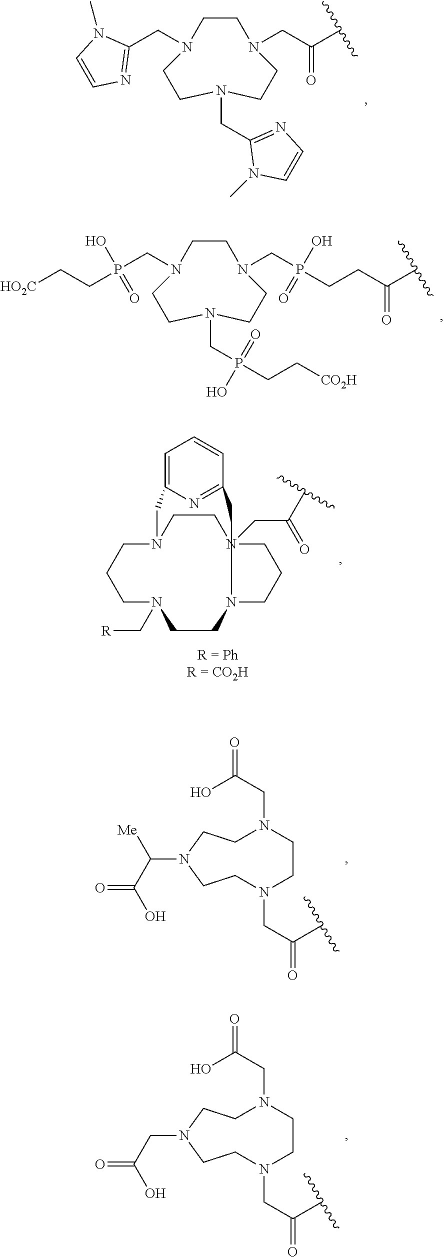

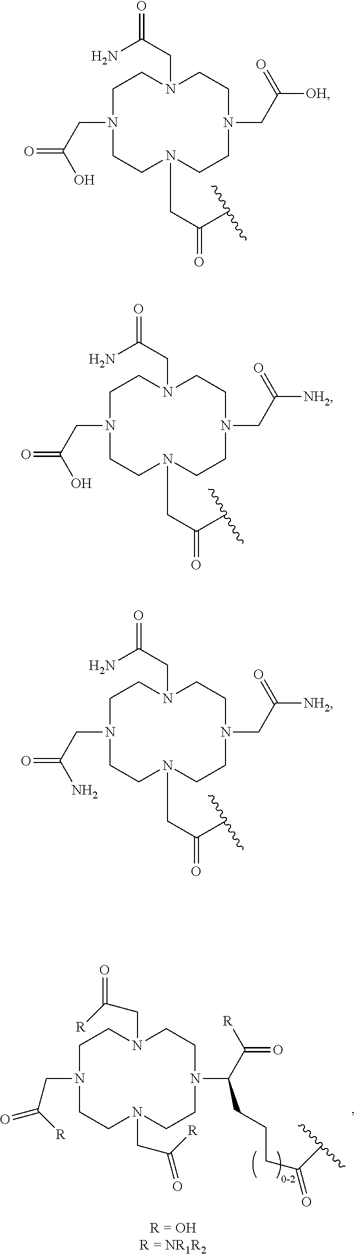

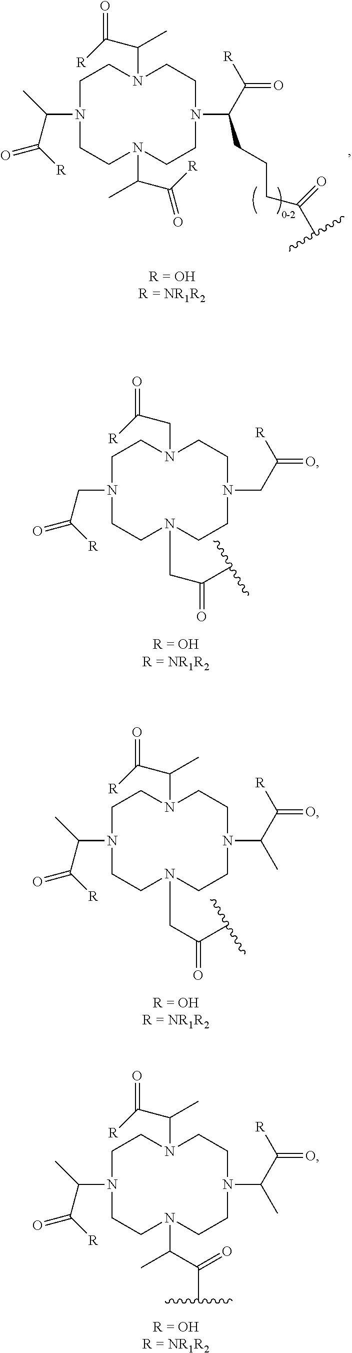

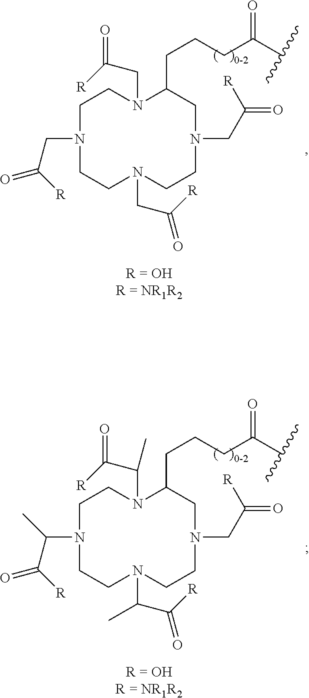

Cp⁴ is a fibrin-binding peptide;
AA is the N-terminal amino acid of the fibrin-binding peptide;
L⁴ is a linker;
y is an integer selected from 0 and 1; and
z is an integer selected from 0 and 1.

In some embodiments of Formula IV, R⁴ is a radioisotope selected from a therapeutic radioisotope and a radioisotope capable of detection using a nuclear imaging technique. In some embodiments, R⁴ is selected from the group consisting of fluorine-18, aluminum fluoride (Al¹⁸F), scandium-43, scandium-44, scandium-47, manganese-51, manganese-52, copper-60, copper-61, copper-62, copper-64, copper-67, gallium-67, gallium-68, yttrium-86, zirconium-89, technetium-99m, yttrium-90, indium-111, iodine-123, iodine-124, iodine-125, iodine-131, terbium-149, terbium-152, samarium-153, terbium-155, terbium-161, holmium-166, lutetium-177, rhenium-188, lead-203, astatine-211, lead-212, bismuth-213, radium-223, actinium-225, and thorium-227.

In some embodiments of Formula IV, R⁴ is a radioisotope that is a therapeutic radioisotope. In some embodiments, the therapeutic radioisotope is selected from the group consisting of scandium-47, copper-67, yttrium-90, iodine-131, samarium-153, terbium-161, holmium-166, lutetium-177, rhenium-188, astatine-211, lead-212, bismuth-213, radium-223, actinium-225, and thorium-227. In some embodiments, the therapeutic isotope is selected from the group consisting of yttrium-90, lutetium-177, and actinium-225. In some embodiments, the therapeutic isotope is yttrium-90. In some embodiments, the therapeutic radioisotope is lutetium-177. In some embodiments, the therapeutic radioisotope is actinium-225.

In some embodiments of Formula IV, R⁴ is a radioisotope capable of detection using a nuclear imaging technique. In some embodiments, the radioisotope is a positron emitting isotope. In some embodiments, the positron emitting isotope is selected from the group consisting of fluorine-18, aluminum fluoride (Al¹⁸F), scandium-43, scandium-44, manganese-51, manganese-52, copper-60, copper-61, copper-62, copper-64, gallium-68, yttrium-86, zirconium-89, iodine-124, terbium-149, and terbium-152. In some embodiments, the positron emitting isotope is selected from the group consisting of fluorine-18, copper-64, and gallium-68. In some embodiments, the positron emitting isotope is fluorine-18. In some embodiments, the positron emitting isotope is copper-64. In some embodiments, the positron emitting isotope is gallium-68. In some embodiments, the positron emitting isotope is copper-64. In some embodiments, the positron emitting isotope is gallium-68. In some embodiments, the radioisotope is a radioisotope suitable for SPECT imaging. In some embodiments, the radioisotope suitable for SPECT imaging is selected from the group consisting of gallium-67, technetium-99m, indium-111, iodine-123, iodine-125, terbium-155, and lead-203.

In some embodiments of Formula IV, C⁴ is selected from the group consisting of:

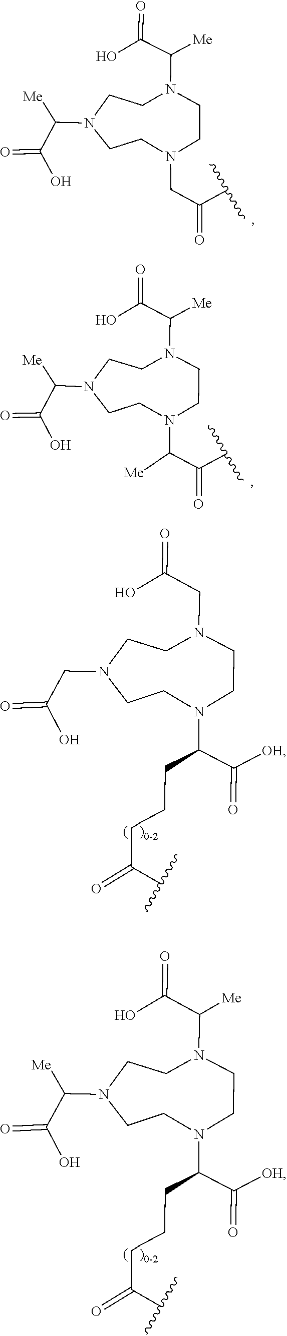

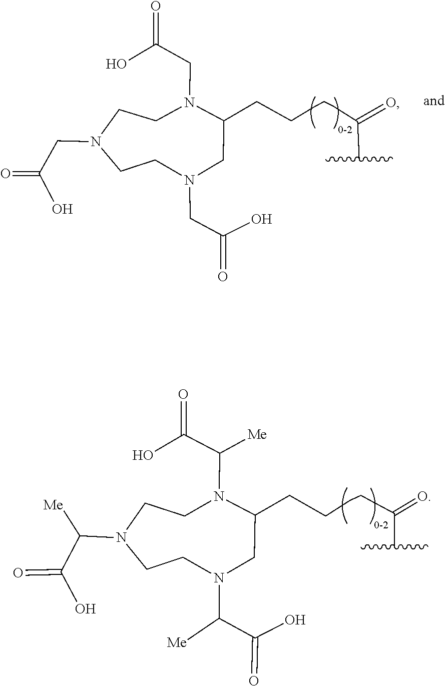

In some embodiments of Formula IV, C⁴ is NODAGA:

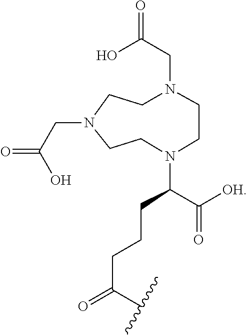 [SMILES: *C(=O)CCC[C@H](C(=O)O)N1CCN(CC(=O)O)CCN(CC(=O)O)CC1]

In some embodiments of Formula IV, C⁴ is selected from the group consisting of:

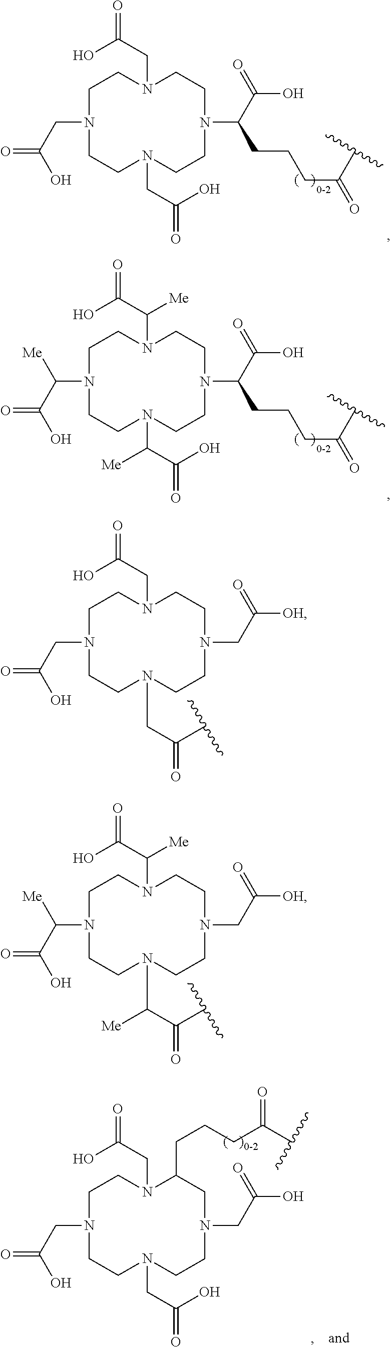

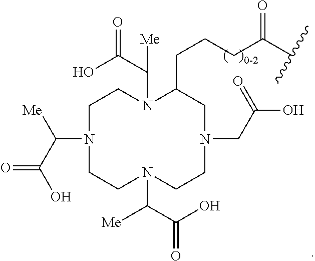

In some embodiments, C⁴ is selected from the group consisting of:

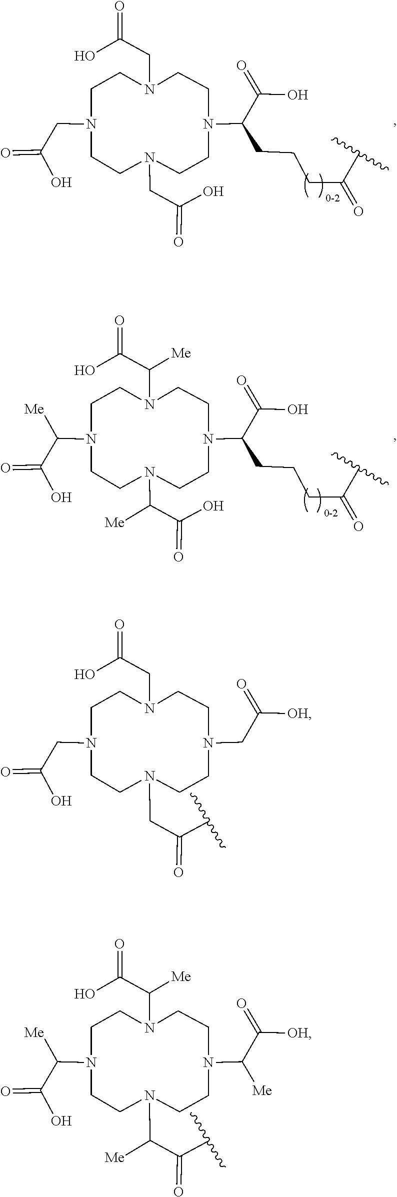

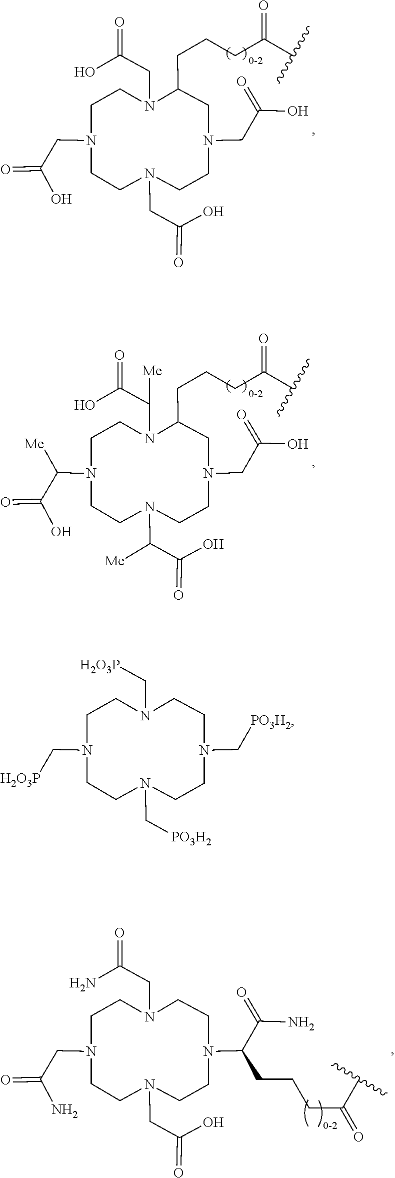

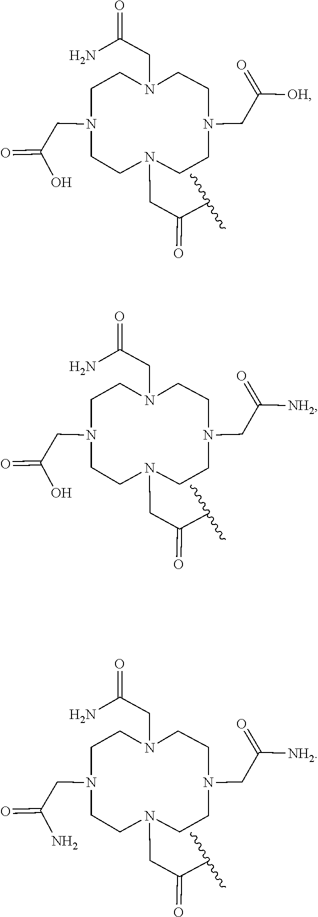 [SMILES: *C(=O)CN1CCN(CC(=O)O)CCN(CC(N)=O)CCN(CC(=O)O)CC1 ; *C(=O)CN1CCN(CC(N)=O)CCN(CC(N)=O)CCN(CC(=O)O)CC1 ; *C(=O)CN1CCN(CC(N)=O)CCN(CC(N)=O)CCN(CC(N)=O)CC1]

In some embodiments of Formula IV, AA-CP⁴ is a fibrin-binding peptide comprising a sequence having at least 80% sequence identity to the polypeptide of SEQ ID NO:1:

(SEQ ID NO: 1)
-y*-X¹-C-Hyp-Y(3-Cl)-X²-L-C-X³-I-X⁴-

wherein each of X¹, X², X³, and X⁴ is independently any amino acid; y* is L-tyrosine or D-tyrosine; and AA represents the N-terminal amino acid. For example, AA-CP⁴ is a fibrin-binding peptide comprising a sequence having at least 85%, at least 90%, at least 92%, at least 95% at least 98%, at least 99% or 100% sequence identity to the polypeptide of SEQ ID NO:1. In some embodiments, AA-CP⁴ is the polypeptide of SEQ ID NO:1 (i.e., it has 100% sequence identity).

In some embodiments, y* is L-tyrosine. In some embodiments, y* is D-tyrosine.

In some embodiments, each of X¹, X², X³, and X⁴ is independently selected from naturally occurring amino acids. For example, each of X¹, X², X³, and X⁴ is independently selected from Ala, Cys, Asp, Glu, Phe, Gly, His, Ile, Lys, Leu, Met, Asn, Pro, Gln, Arg, Ser, Thr, Val, Trp, and Tyr. In some embodiments, each of X¹, X², X³, and X⁴ is independently selected from the D-configuration of the naturally occurring amino acids. For example, each of X¹, X², X³, and X⁴ is independently selected from D-Ala, D-Cys, D-Asp, D-Glu, D-Phe, D-His, D-Ile, D-Lys, D-Leu, D-Met, D-Asn, D-Pro, D-Gln, D-Arg, D-Ser, D-Thr, D-Val, D-Trp, and D-Tyr. In some embodiments, X¹ is Glu. In some embodiments, X¹ is D-His. In some embodiments, X² is Gly. In some embodiments, X² is Asp. In some embodiments, X² is D-Asp. In some embodiments, X³ is His. In some embodiments, X³ is Tyr. In some embodiments, X⁴ is Gln. In some embodiments, X⁴ is D-Gln. In some embodiments, X⁴ is Leu. In some embodiments, X⁴ is D-Leu.

In some embodiments, each of X¹, X², X³, and X⁴ is independently selected from non-naturally occurring amino acids. For example, each of X¹, X², X³, and X⁴ is independently selected from Hyp, D-Hyp, Tyr-3-Cl, and D-Tyr-3-Cl.

In some embodiments of Formula IV, AA-CP⁴ is a fibrin-binding peptide, wherein AA represents the N-terminal amino acid, comprising a polypeptide having at least 80% (e.g., at least 85%, at least 90%, at least 92%, at least 95%, at least 98%, at least 99%, or 100%) sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments of Formula IV, AA-CP⁴ is a fibrin-binding peptide, wherein AA represents the N-terminal amino acid, having at least 80% (e.g., at least 85%, at least 90%, at least 92%, at least 95%, at least 98%, at least 99%, or 100%) sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |
| 6 | -y-e-C-Hyp-Y(3-Cl)-G-L-C-Y-I-Q- |
| 7 | -Y-e-C-Hyp-Y(3-Cl)-G-L-C-Y-I-Q- |
| 8 | -Y-e-C-Hyp-Y(3-Cl)-G-L-C-H-I-l- |
| 9 | -y-e-C-Hyp-Y(3-Cl)-G-L-C-Y-I-l- |
| 10 | -Y-e-C-Hyp-Y(3-Cl)-G-L-C-Y-I-l- |
| 11 | -y-e-C-Hyp-Y(3-Cl)-G-L-C-Y-I-q- |
| 12 | -Y-e-C-Hyp-Y(3-Cl)-D-L-C-Y-I-Q- |
| 13 | -Y-e-C-Hyp-Y(3-Cl)-G-L-C-Y-I-q- |
| 14 | -Y-e-C-Hyp-Y(3-Cl)-d-L-C-Y-I-Q- |
| 15 | -Y-h-C-Hyp-Y(3-Cl)-G-L-C-Y-I-Q- |

In some embodiments of Formula IV, AA-CP⁴ is a fibrin-binding peptide, wherein AA represents the N-terminal amino acid, having at least 80% (e.g., at least 85%, at least 90%, at least 92%, at least 95%, at least 98%, at least 99%, or 100%) sequence identity to a polypeptide selected from the group consisting of:

| SEQ ID | |
|---|---|
| NO: | Sequence |
| 2 | -y-e-C-Hyp-Y(3-Cl)-G-L-C-H-I-Q- |
| 3 | -y-e-C-Hyp-Y(3-Cl)-G-L-C-H-I-l- |
| 4 | -Y-e-C-Hyp-Y(3-Cl)-G-L-C-H-I-Q- |
| 5 | -Y-e-C-Hyp-Y(3-Cl)-G-L-C-H-I-q- |

In some embodiments of Formula IV, AA-CP⁴ is a fibrin-binding peptide, wherein AA represents the N-terminal amino acid, having at least 80% (e.g., at least 85%, at least 90%, at least 92%, at least 95%, at least 98%, at least 99%, or 100%) sequence identity to a polypeptide selected from the group consisting of:

| SEQ ID | |
|---|---|
| NO: | Sequence |
| 5 | -Y-e-C-Hyp-Y(3-Cl)-G-L-C-H-I-q- |

In some embodiments of Formula IV, the [(L⁴)_{z}-(AA)] moiety represents the N-terminal amino acid of the fibrin-binding peptide CP⁴ optionally modified with a linker, where AA is the N-terminal amino acid of the fibrin-binding peptide and L⁴ is the optional linker. The linker L⁴ can be any compound comprising a functional group that can form a covalent bond with the N-terminus of an amino acid, such as the N-terminus of the fibrin-binding peptide CP⁴.

In some embodiments, L⁴ is selected from the group consisting of C₁-C₆ alkyl, 6-10 membered aryl, 5-10 membered heteroaryl, C₁-C₆ alkyl-C(O)—, 6-10 membered aryl-C(O)—, and 5-10 membered heteroaryl-C(O)—. In some embodiments, L⁴ is 5-6 membered heteroaryl. In some embodiments, L⁴ is a pyridinyl group. In some embodiments, L⁴ is 5-6 membered heteroaryl-C(O)—. In some embodiments, L⁴ is (pyridinyl)-C(O)—. In some embodiments, L⁴ is (pyridin-3-yl)-C(O)—.

In some embodiments of the compound of Formula IV, the [(C⁴)_{y}—(R⁴))] moiety binds to the [(L⁴)_{z}-(AA)] moiety through the L⁴ group, where the chelating moiety C⁴ binds to the L⁴ group. In some embodiments, the [(C⁴)_{y}—(R⁴))] moiety binds to the [(L⁴)_{z}-(AA)] moiety through the L⁴ group, where the radioisotope R⁴ binds to the L⁴ group. In some embodiments, the [(C⁴)_{y}—(R⁴))] moiety binds to the [(L⁴)_{z}-(AA)] moiety through the AA group, where the radioisotope R⁴ binds to the AA group. In some embodiments, the [(C⁴)_{y}—(R⁴))] moiety binds to the [(L⁴)_{z}-(AA)] moiety through the AA group, where the chelating moiety C⁴ binds to the AA group.

In some embodiments of Formula IV, y is 0. In some embodiments, y is 1.

In some embodiments of Formula IV, z is 0. In some embodiments, z is 1.

In some embodiments of Formula IV, y is 0 and z is 0. In some embodiments, y is 0 and z is 1. In some embodiments, 1 is y, and z is 0. In some embodiments, 1 is y and z is 1.

In some embodiments, the compound of Formula IV is a compound of Formula IVa:

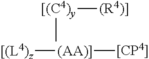

or a pharmaceutically acceptable salt thereof, wherein R⁴ is a radioisotope that is capable of being chelated by the chelating moiety C⁴.

In some embodiments, R⁴ is selected from the group consisting of aluminum-fluoride (Al¹⁸F), scandium-43, scandium-44, scandium-47, manganese-51, manganese-52, copper-60, copper-61, copper-62, copper-64, copper-67, gallium-67, gallium-68, yttrium-86, zirconium-89, technetium-99m, yttrium-90, indium-111, terbium-149, terbium-152, samarium-153, terbium-155, terbium-161, holmium-166, lutetium-177, rhenium-188, lead-203, lead-212, bismuth-213, radium-223, actinium-225, and thorium-227.

In some embodiments of the compound of Formula IVa, z is 0 and the [(C⁴)—(R⁴)] moiety binds to the [AA] moiety via the chelating moiety C⁴. In some embodiments, the chelating moiety C⁴ forms an amide bond with the AA group.

In some embodiments of the compound of Formula IVa, z is 1 and the [(C⁴)—(R⁴)] moiety binds to the [(L⁴)-(AA)] moiety via the L⁴ group, where the chelating moiety C⁴ binds to the L⁴ group.

In some embodiments, the compound of Formula IV is a compound of Formula IVb:

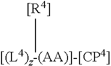

or a pharmaceutically acceptable salt thereof, wherein R⁴ is a radioisotope that is capable of covalently binding to the linker L⁴, the N-terminal amino acid of the fibrin-binding peptide AA, or both.

In some embodiments, R⁴ is selected from the group consisting of fluorine-18, iodine-123, iodine-124, iodine-125, iodine-131, and astatine-211.

In some embodiments of the compound of Formula IVb, z is 0 and the [R⁴] moiety binds directly to the [AA] moiety.

In some embodiments of the compound of Formula IVb, z is 1 and the [R⁴] moiety binds to the [(L⁴)-(AA)] moiety via the L⁴ group. In some embodiments, the R⁴ moiety forms a covalent bond with the L⁴ group. In some embodiments, R⁴ is fluorine-18.

In some embodiments, the compound of Formula IV is a compound of Formula IVc:

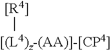

or a pharmaceutically acceptable salt thereof, where the [R⁴] moiety binds to the [(L⁴)-(AA)] moiety via a covalent bond with the L⁴ group, wherein R⁴ is a radioisotope that is capable of covalently binding to the linker L⁴, the N-terminal amino acid of the fibrin-binding peptide AA, or both.

In some embodiments, R⁴ is selected from the group consisting of fluorine-18, iodine-123, iodine-124, iodine-125, iodine-131, and astatine-211. In some embodiments, R⁴ is fluorine-18.

In some embodiments, the compound of Formula IV is a compound of Formula IVd:

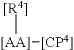

or a pharmaceutically acceptable salt thereof, where the [R⁴] moiety binds directly to the [AA] moiety, wherein R⁴ is a radioisotope that is capable of covalently binding to the linker L⁴, the N-terminal amino acid of the fibrin-binding peptide AA, or both.

In some embodiments, R⁴ is selected from the group consisting of fluorine-18, iodine-123, iodine-124, iodine-125, iodine-131, and astatine-211.

In some embodiments of Formula IV, the compound is selected from the group consisting of:

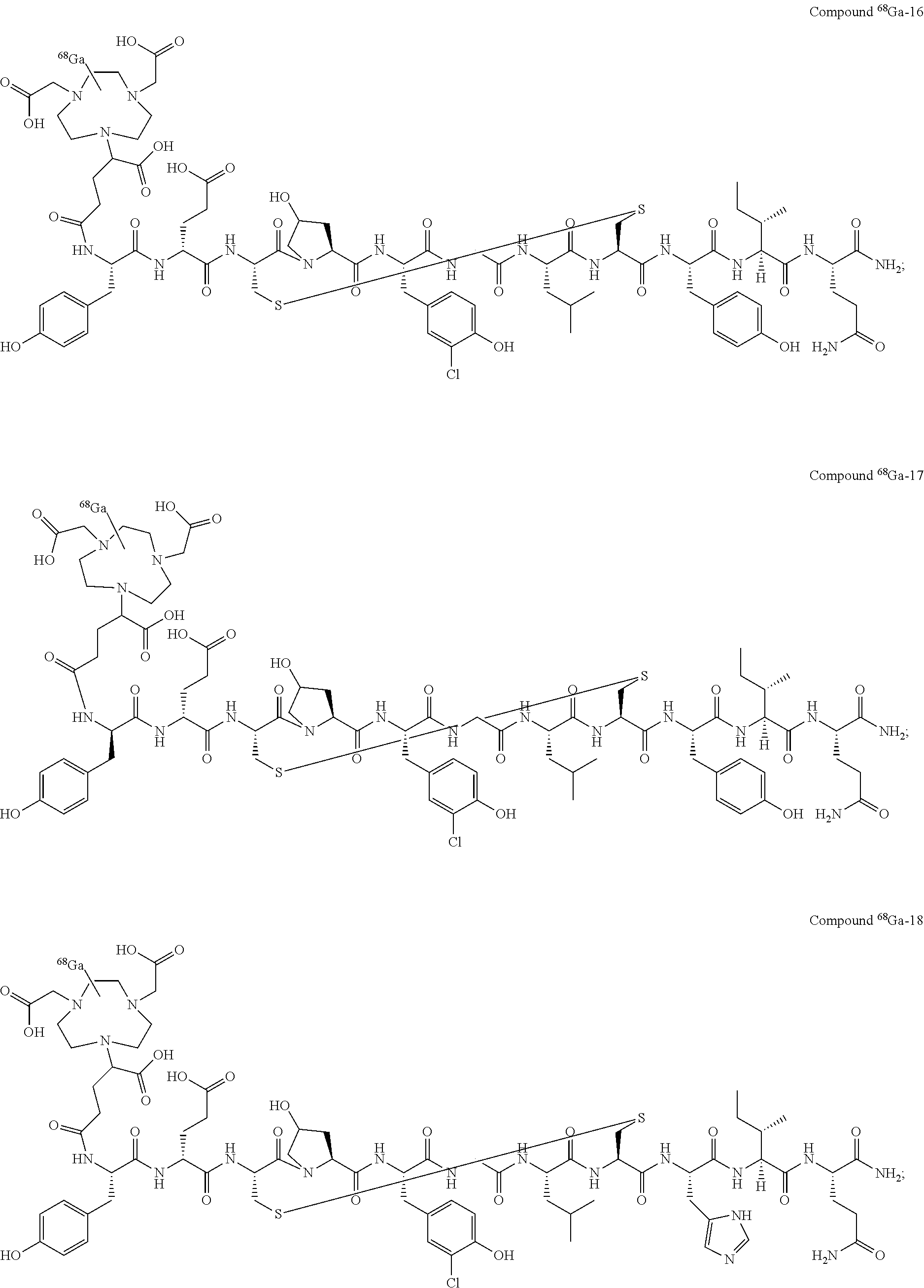

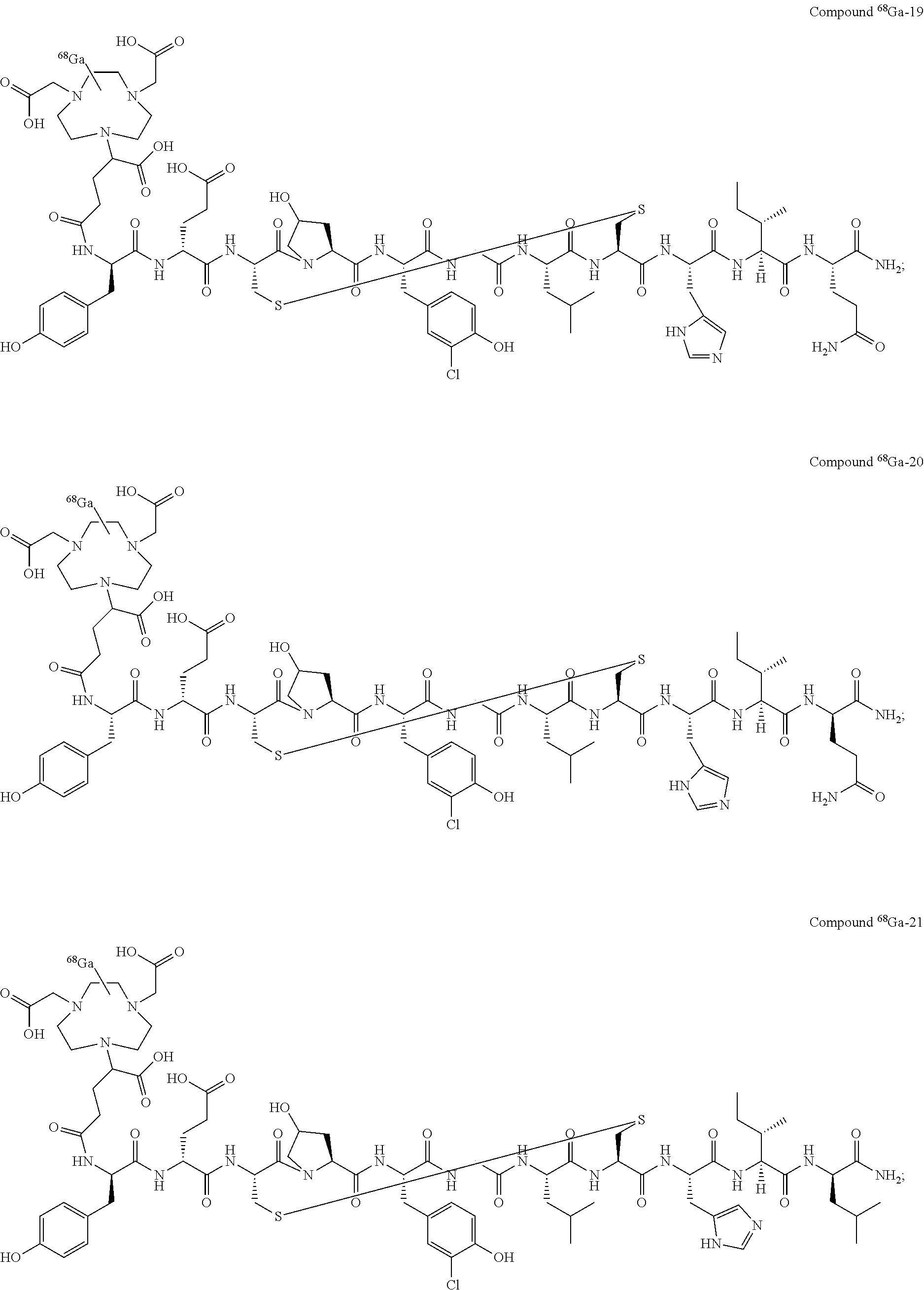

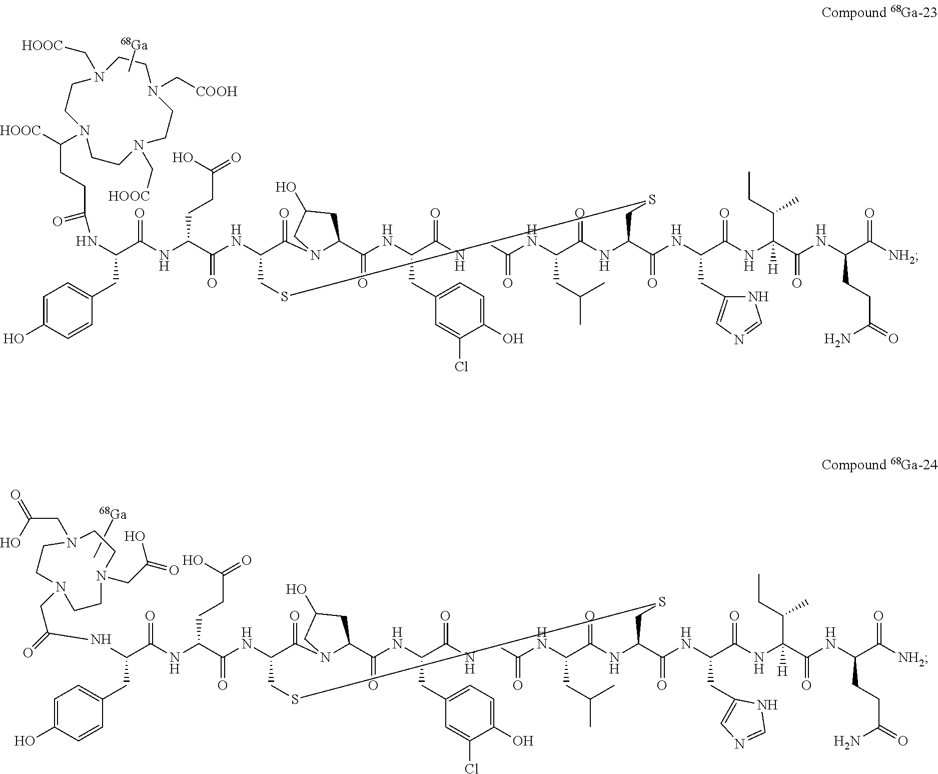

or a pharmaceutically acceptable salt thereof.

In some embodiments of Formula IV, the compound is selected from the group consisting of:

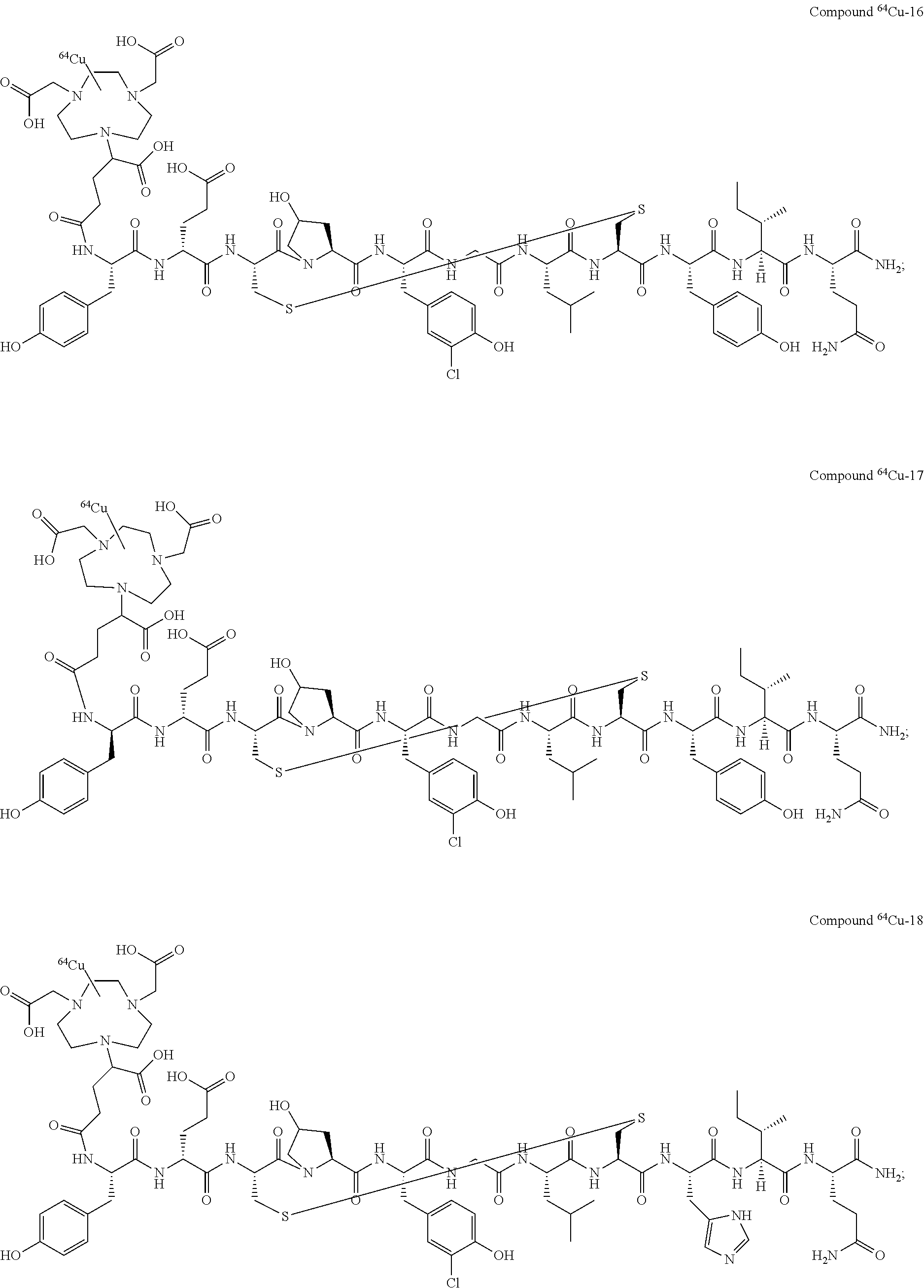

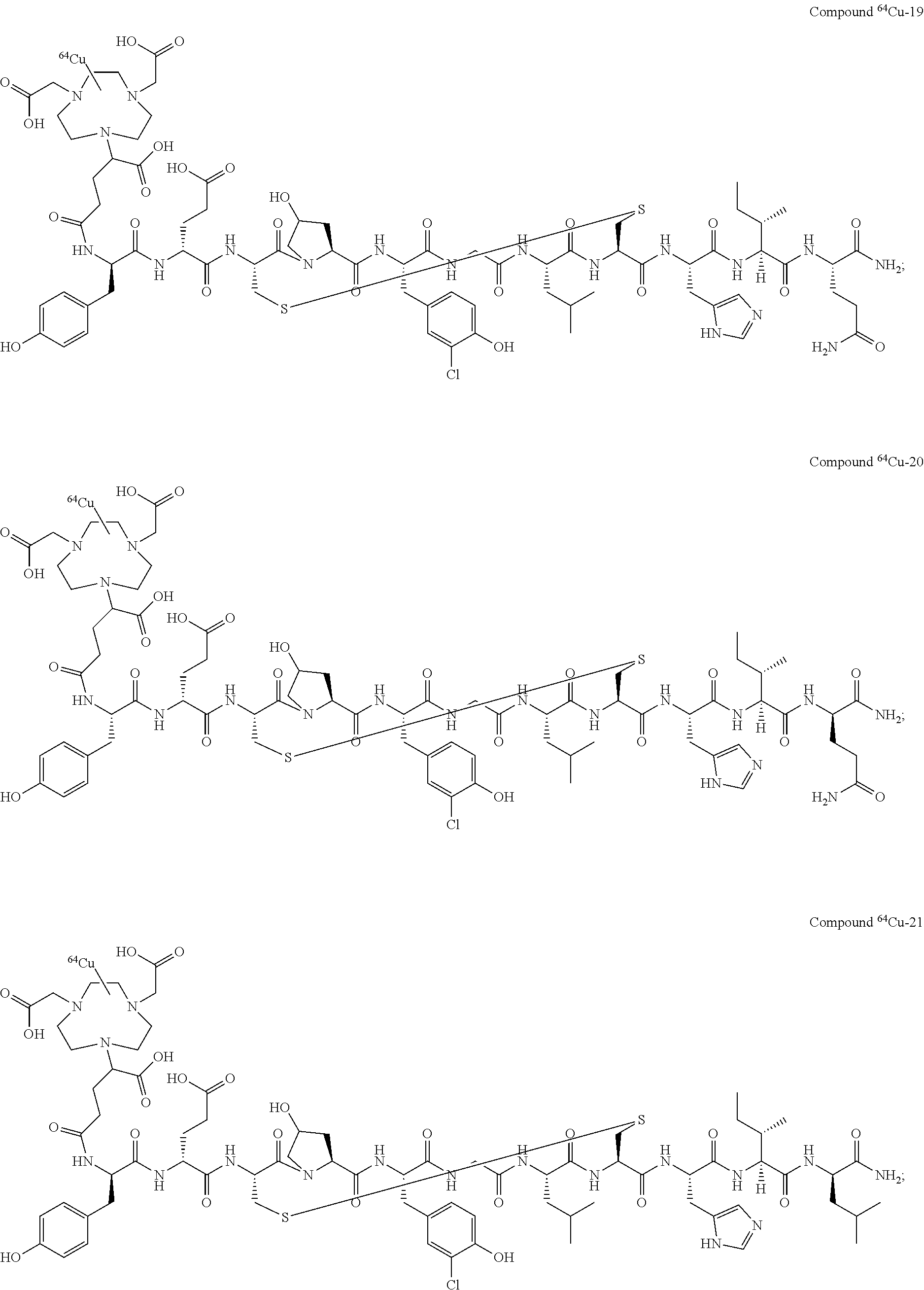

or a pharmaceutically acceptable salt thereof.

In some embodiments of Formula IV, the compound is:

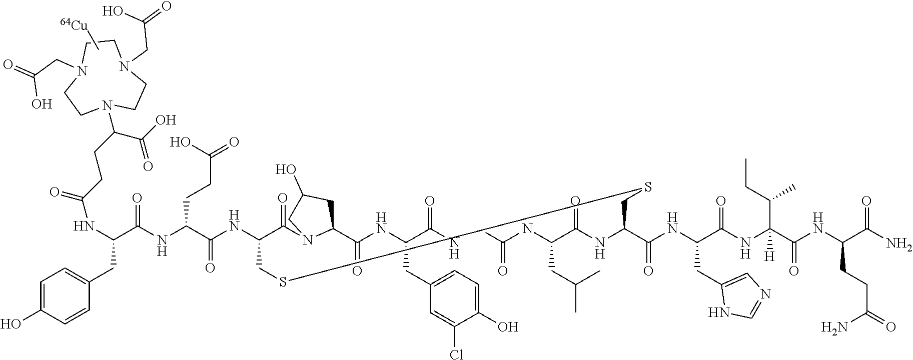

or a pharmaceutically acceptable salt thereof.

In some embodiments of Formula IV, the compound is:

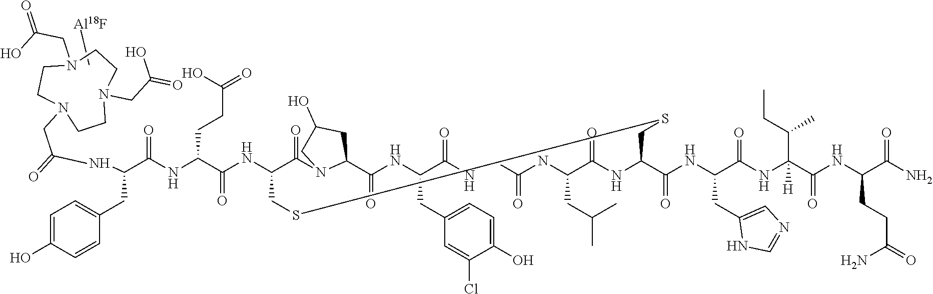

or a pharmaceutically acceptable salt thereof.

In some embodiments of Formula IV, the compound is selected from the group consisting of:

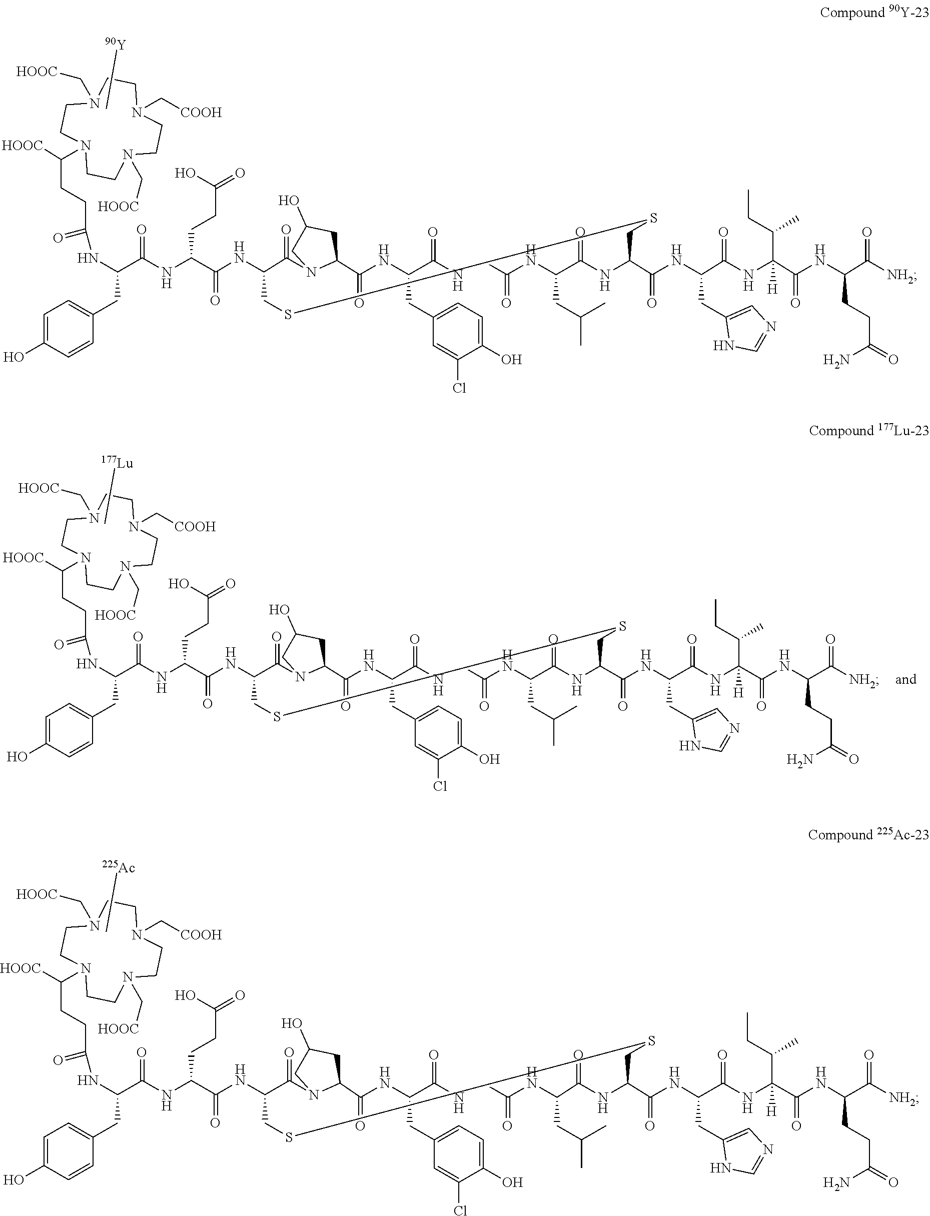

or a pharmaceutically acceptable salt thereof.

In some embodiments of Formula IV, the compound is selected from the group consisting of:

| Compound | N-terminus | Sequence |
|---|---|---|
| 25 | 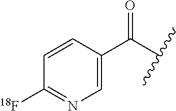 [SMILES: *C(=O)c1ccc([18F])nc1] | -y-e-C-Hyp-Y(3-Cl)-G-L-C-H-I-Q- |
| 26 | 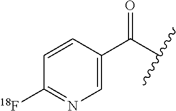 [SMILES: *C(=O)c1ccc([18F])nc1] | -y-e-C-Hyp-Y(3-Cl)-G-L-C-H-I-1- |
| 27 | 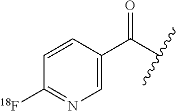 [SMILES: *C(=O)c1ccc([18F])nc1] | -Y-e-C-Hyp-Y(3-Cl)-G-L-C-H-I-Q- |
| 28 | 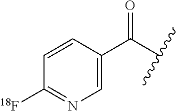 [SMILES: *C(=O)c1ccc([18F])nc1] | -Y-e-C-Hyp-Y(3-Cl)-G-L-C-H-I-q- |
| 29 | 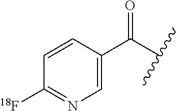 [SMILES: *C(=O)c1ccc([18F])nc1] | -y-e-C-Hyp-Y(3-Cl)-G-L-C-Y-I-Q- |
| 30 | 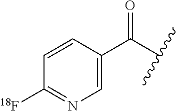 [SMILES: *C(=O)c1ccc([18F])nc1] | -Y-e-C-Hyp-Y(3-Cl)-G-L-C-Y-I-Q- |
| 31 | 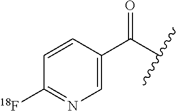 [SMILES: *C(=O)c1ccc([18F])nc1] | -Y-e-C-Hyp-Y(3-Cl)-G-L-C-H-I-1- |
| 32 | 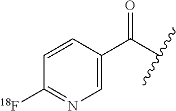 [SMILES: *C(=O)c1ccc([18F])nc1] | -y-e-C-Hyp-Y(3-Cl)-G-L-C-Y-I-1- |
| 33 | 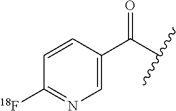 [SMILES: *C(=O)c1ccc([18F])nc1] | -Y-e-C-Hyp-Y(3-Cl)-G-L-C-Y-I-1- |
| 34 | 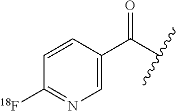 [SMILES: *C(=O)c1ccc([18F])nc1] | -y-e-C-Hyp-Y(3-Cl)-G-L-C-Y-I-q- |
| 35 | 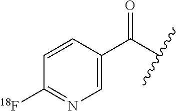 [SMILES: *C(=O)c1ccc([18F])nc1] | -Y-e-C-Hyp-Y(3-Cl)-D-L-C-Y-I-Q- |
| 36 | 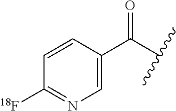 [SMILES: *C(=O)c1ccc([18F])nc1] | -Y-e-C-Hyp-Y(3-Cl)-G-L-C-Y-I-q- |
| 37 | 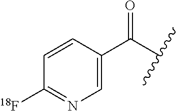 [SMILES: *C(=O)c1ccc([18F])nc1] | -Y-e-C-Hyp-Y(3-Cl)-d-L-C-Y-I-Q- |
| 38 | 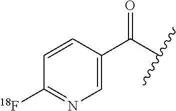 [SMILES: *C(=O)c1ccc([18F])nc1] | -Y-h-C-Hyp-Y(3-Cl)-G-L-C-Y-I-Q- |

Also provided in the present disclosure is a compound of Formula V:

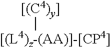

or a pharmaceutically acceptable salt thereof, wherein C⁴, L⁴, AA, CP⁴, y, and z are as described in the present disclosure for compounds of Formula IV.

In some embodiments, the compound of Formula V is a compound selected from the group consisting of:

| Compound | Sequence |
|---|---|
| 16 | NODAGA-Y-e-C-Hyp-Y(3-Cl)-G-L-C-Y-I-Q-NH₂ |
| 17 | NODAGA-y-e-C-Hyp-Y(3-Cl)-G-L-C-Y-I-Q-NH₂ |
| 18 | NODAGA-Y-e-C-Hyp-Y(3-Cl)-G-L-C-H-I-Q-NH₂ |
| 19 | NODAGA-y-e-C-Hyp-Y(3-Cl)-G-L-C-H-I-Q-NH₂ |
| 20 | NODAGA-Y-e-C-Hyp-Y(3-Cl)-G-L-C-H-I-q-NH₂ |
| 21 | NODAGA-y-e-C-Hyp-Y(3-Cl)-G-L-C-H-I-I-NH₂ |
| 22 | NODAGA-y-e-c-Hyp-Y(3-Cl)-G-L-C-H-I-q-NH₂ |
| 23 | DOTAGA-Y-e-C-Hyp-Y(3-Cl)-G-L-C-H-I-q-NH₂ |
| 24 | NOTA-Y-e-C-Hyp-Y(3-Cl)-G-L-C-H-I-q-NH₂ |

### Pharmaceutically Acceptable Derivatives and Compositions

In some embodiments, the compounds of the present disclosure can be formulated as a pharmaceutical composition. In some embodiments, a pharmaceutical composition comprises a compound of Formula I, or a pharmaceutically acceptable salt thereof, and a pharmaceutically acceptable excipient. In some embodiments, a pharmaceutical composition comprises a compound of Formula II, or a pharmaceutically acceptable salt thereof, and a pharmaceutically acceptable excipient. In some embodiments, a pharmaceutical composition comprises a compound of Formula III, or a pharmaceutically acceptable salt thereof, and a pharmaceutically acceptable excipient. In some embodiments, a pharmaceutical composition comprises a compound of Formula IV, or a pharmaceutically acceptable salt thereof, and a pharmaceutically acceptable excipient.

As used herein, the compounds can include pharmaceutically acceptable derivatives thereof. “Pharmaceutically acceptable” means that the compound or composition can be administered to an animal without unacceptable adverse effects. A “pharmaceutically acceptable derivative” means any pharmaceutically acceptable salt, ester, or salt of an ester of the compounds of the present disclosure that, upon administration to a recipient, is capable of providing (directly or indirectly) the compounds or an active metabolite or residue thereof.

Other derivatives are those that increase the bioavailability of the compounds when such compounds are administered to a mammal (e.g., by allowing an orally administered compound to be more readily absorbed into the blood), or which enhance delivery of the parent compound to a biological compartment (e.g., the brain or lymphatic system) thereby increasing the exposure relative to the parent species.

Pharmaceutically acceptable salts of the compounds of the present disclosure include counter ions derived from pharmaceutically acceptable inorganic and organic acids and bases known in the art. For example, alkali and alkaline earth metal cations; sodium; primary, secondary and tertiary amines such as ethanolamine, diethanolamine, morpholine, glucamine, N,N-dimethylglucamine, N-methylglucamine; and amino acids such as lysine, arginine and ornithine.

Conventional excipients, such as binding agents, fillers, acceptable wetting agents, tableting lubricants, and disintegrants can be used in tablets and capsules for oral administration. Liquid preparations for oral administration can be in the form of solutions, emulsions, aqueous or oily suspensions, and syrups. Alternatively, the oral preparations can be in the form of dry powder that can be reconstituted with water or another suitable liquid vehicle before use. Additional additives, such as suspending or emulsifying agents, non-aqueous vehicles (including edible oils), preservatives, and flavorings and colorants can be added to the liquid preparations. Parenteral dosage forms can be prepared by dissolving the compound of Formula I, II, III, or IV, or a pharmaceutically acceptable salt thereof in a suitable liquid vehicle and filter sterilizing the solution before filling and sealing an appropriate vial or ampoule. These are just a few examples of the many appropriate methods well known in the art for preparing dosage forms.

A compound of Formula I, II, III, or IV, or pharmaceutically acceptable salt thereof can be formulated into pharmaceutical compositions using techniques well known to those in the art. Suitable pharmaceutically acceptable carriers, outside those mentioned herein, are known in the art; for example, see Remington, The Science and Practice of Pharmacy, 20th Ed., 2000, Lippincott Williams & Wilkins, (Editors: Gennaro, A. R., et al.).

Pharmaceutical compositions described herein can be administered by any route, including both oral and parenteral administration. Parenteral administration includes, but is not limited to, subcutaneous, intravenous, intraarterial, interstitial, intrathecal, and intracavity administration. When administration is intravenous, pharmaceutical compositions may be given as a bolus, as two or more closes separated in time, or as a constant or non-linear flow infusion. Thus, compositions of the present disclosure can be formulated for any route of administration.

In some embodiments, pharmaceutical compositions for intravenous administration are solutions in sterile isotonic aqueous buffer. In some embodiments, the composition can also include one or more of a solubilizing agent, a stabilizing agent, and a local anesthetic (e.g., lidocaine) to ease pain at the site of the injection. In some embodiments, the composition for intravenous administration includes sucrose (e.g., 80 millimolar). In some embodiments, the ingredients will be supplied either separately, e.g. in a kit, or mixed together in a unit dosage form, for example, as a dry lyophilized powder or water free concentrate. The compositions can be stored in a hermetically sealed container such as an ampule or sachette indicating the quantity of active agent in activity units. Where the composition is administered by infusion, it can be dispensed with an infusion bottle containing sterile pharmaceutical grade “water for injection,” saline, or other suitable intravenous fluids. Where the composition is to be administered by injection, an ampule of sterile water for injection or saline can be provided as a component of the kit so that the ingredients may be mixed prior to administration.

The pharmaceutical compositions of the present disclosure containing a compound of Formula I, II, III, or IV, or pharmaceutically acceptable salt thereof, can further contain one or more other ingredients. Examples of such ingredients include, but are not limited to, pH adjusters, stabilizing agents, decontaminating agents, and isotonicity agents. In some embodiments, the pharmaceutical composition contains one or more of acetic acid, sodium acetate, sodium hydroxide, gentisic acid, ascorbic acid, diethylene triamine pentaacetic acid (DPTA), and sodium chloride. In some embodiments, the pharmaceutical composition contains water for injection.

In some embodiments, the pharmaceutical composition comprising a compound of Formula I, II, III, or IV, or a pharmaceutically acceptable salt thereof, and a pharmaceutically acceptable excipient, comprises a radical scavenger. The radical scavenger can be used to prevent radiolysis. Radiolysis is the process in which the ionization of oxygen or water molecules induced by the radionuclide leads the formation of other reactive species, such as superoxide, hydrogen peroxide, hydrogen radicals, ozone and hydroxyl radicals. These reactive species can further cause damage to DNA and other cellular structures. In some embodiments, the radical scavenger is an antioxidant selected from carnosic acid, green tea extract, apigenin, diosmine, rosmarinic acid, lipoic acid, beta carotene, L-ascorbic acid (vitamin C), N-acetyl cysteine (NAC), δ-tocopherol, rutin, amifostine, resveratrol, gentisic acid, and gallic acid. In some embodiments, the radical scavenger is an antioxidant selected from gallic acid, L-ascorbic acid and N-acetyl cysteine (NAC).

In some embodiments, the compositions of the present disclosure are administered to the patient in the form of an injectable composition. In some embodiments, the method of administering a compound is parenterally, meaning intravenously, intra-arterially, intrathecally, interstitially or intracavitarilly. Pharmaceutical compositions of this invention can be administered to animals including humans in a manner similar to other diagnostic or therapeutic agents. The dosage to be administered, and the mode of administration will depend on a variety of factors including age, weight, sex, condition of the patient and genetic factors, and will ultimately be decided by medical personnel subsequent to experimental determinations of varying dosage followed by imaging as described herein.

### Methods of Imaging Fibrin

The compounds and compositions of the present disclosure can be used to image fibrin. In some embodiments, the fibrin is present in a tumor. In some embodiments, the tumor is cancerous. In some embodiments, the fibrin is present in a thrombus. In some embodiments, the fibrin is associated with neuroinflammation. In some embodiments, the neuroinflammation is associated with Alzheimer's disease, multiple sclerosis, or traumatic brain injury.

In some embodiments, a method for imaging fibrin in a mammal comprises administering to the mammal an effective amount of a compound of Formula I, Formula II, Formula III, or Formula IV, or a pharmaceutically acceptable salt thereof; acquiring an image of the fibrin of the mammal using a nuclear imaging technique and acquiring an anatomical image of the mammal using magnetic resonance imaging (MRI); and overlaying said images to localize the fibrin within the anatomical image of the mammal.

In some embodiments, a method for imaging fibrin in a mammal comprises administering to the mammal an effective amount of a pharmaceutical composition comprising an compound of Formula I, Formula II, Formula III, or Formula IV, or a pharmaceutically acceptable salt thereof, and a pharmaceutically acceptable excipient; acquiring an image of the fibrin of the mammal using a nuclear imaging technique and acquiring an anatomical image of the mammal using magnetic resonance imaging; and overlaying said images to localize the fibrin within the anatomical image of the mammal.

In some embodiments of the method for imaging fibrin, the image of the fibrin of the mammal using a nuclear imaging technique and the image of the mammal using magnetic resonance imaging are acquired simultaneously. In some embodiments of the method for imaging fibrin, the image of the fibrin of the mammal using a nuclear imaging technique is acquired first, and the image of the mammal using magnetic resonance imaging is acquired second. In some embodiments of the method for imaging fibrin, the image of the mammal using magnetic resonance imaging is acquired first, and the image of the fibrin of the mammal using a nuclear imaging technique is acquired second.

In some embodiments of the method for imaging fibrin, the nuclear imaging technique is single photon emission computed tomography (SPECT).

In some embodiments of the method for imaging fibrin, the nuclear imaging technique is positron emission tomography (PET). In some embodiments of the method for imaging fibrin, the nuclear imaging technique is positron emission tomography in combination with computed tomography (PET-CT).

In some embodiments of the method for imaging fibrin, the mammal is a human. In some embodiments of the method for imaging fibrin, the mammal is a rat. In some embodiments of the method for imaging fibrin, the mammal is a dog.

In some embodiments of the method for imaging fibrin, the method further comprises administering to the mammal an effective amount of a second compound or composition. In some embodiments, the second compound or composition does not target fibrin.

In some embodiments, the second compound or composition containing a second compound is a second imaging agent. In some embodiments, the second imaging agent comprises an MRI imaging agent. In some embodiments, the second imaging agent is an MRI imaging agent. For example, gadoteridol, gadopentetate, gadobenate, gadoxetic acid, gadodiamide, gadoversetamide, and gadofosveset; or a CT imaging agent selected from the group consisting of iopamidol, iohexol, ioxilan, iopromide, iodixanol, ioxaglate, metrizoate, and diatrizoate.

In some embodiments, the second compound or composition containing a second compound comprises a therapeutic radioisotope. In some embodiments, the therapeutic radioisotope is selected from the group consisting of scandium-47, copper-67, yttrium-90, iodine-131, samarium-153, terbium-161, holmium-166, lutetium-177, rhenium-188, astatine-211, lead-212, bismuth-213, radium-223, actinium-225, and thorium-227. In some embodiments, the therapeutic isotope is selected from the group consisting of yttrium-90, lutetium-177, and actinium-225. In some embodiments, the therapeutic isotope is yttrium-90. In some embodiments, the therapeutic isotope is lutetium-177. In some embodiments, the therapeutic isotope is actinium-225.

### Image Overlay

Overlaying of images can be done by various means known in the art. See, for example, U.S. Pat. Nos. 7,412,279; 7,110,616; 6,898,331; 6,549,798; and 5,672,877; Rudd, J. H F. et al., J. Nucl. Med. 2008 49(6): 871-878; Slomka, P. J. et al., J. Nucl. Med. 2009 50: 1621-1630; and Jupp, B. and O'Brien, T. J., Epilepsia 2007 49: 82-89. In some embodiments, the first and second image data sets can be overlaid to determine the presence of the fibrin within the mammal. For example, the first and second image data sets can be combined to produce a third data set that includes an image of the fibrin target and an image of anatomical region where the fibrin is located. The third data set is capable of indicating the location of the fibrin, if present, within the mammal. If desired, the third data set may be displayed on a display device in order to indicate the location of the stationary target within the vascular system. The third data set may also indicate the size of the stationary target within the mammal.

### Methods of Treatment

Also provided in the present disclosure are methods for treating a disease or condition associated with the presence of fibrin. In some embodiments, the disease or condition associated with the presence of fibrin is a cardiovascular disease. In some embodiments, the disease or condition associated with the presence of fibrin is cancer.

In some embodiments, the method for treating a disease or condition associated with the presence of fibrin in a mammal comprises administering to the mammal an effective amount of a compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, wherein the compound or pharmaceutical composition contains a therapeutic radioisotope. In some embodiments, the method for treating a disease or condition associated with the presence of fibrin in a mammal comprises administering to the mammal a pharmaceutical composition comprising a compound of Formula I, or a pharmaceutically acceptable salt thereof, wherein the compound contains a therapeutic radioisotope. In some embodiments, the method for treating a disease or condition associated with the presence of fibrin in a mammal comprises administering to the mammal a pharmaceutical composition comprising a compound of Formula II, or a pharmaceutically acceptable salt thereof, wherein the compound contains a therapeutic radioisotope. In some embodiments, the method for treating a disease or condition associated with the presence of fibrin in a mammal comprises administering to the mammal a pharmaceutical composition comprising a compound of Formula III, or a pharmaceutically acceptable salt thereof, wherein the compound contains a therapeutic radioisotope. In some embodiments, the method for treating a disease or condition associated with the presence of fibrin in a mammal comprises administering to the mammal a pharmaceutical composition comprising a compound of Formula IV, or a pharmaceutically acceptable salt thereof, wherein the compound contains a therapeutic radioisotope. In some embodiments, the disease or condition associated with the presence of fibrin is a cardiovascular disease or condition. In some embodiments, the disease or condition associated with the presence of fibrin is a cancer. In some embodiments, the disease or condition associated with the presence of fibrin is a cerebrovascular disease (e.g., brain aneurysm, carotid stenosis, vertebral stenosis and stroke). In some embodiments, the mammal is a rat, a mouse, a dog or a pig. In some embodiments, the mammal is a human.

Also provided in the present disclosure is a method of treating cancer in a mammal. In some embodiments, the method comprises administering to the mammal an effective amount of a compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, wherein the compound or pharmaceutical composition contains a therapeutic radioisotope. In some embodiments, the method for treating cancer in a mammal comprises administering to the mammal a pharmaceutical composition comprising a compound of Formula I, or a pharmaceutically acceptable salt thereof, wherein the compound contains a therapeutic radioisotope. In some embodiments, the method for treating cancer in a mammal comprises administering to the mammal a pharmaceutical composition comprising a compound of Formula II, or a pharmaceutically acceptable salt thereof, wherein the compound contains a therapeutic radioisotope. In some embodiments, the method for treating cancer in a mammal comprises administering to the mammal a pharmaceutical composition comprising a compound of Formula III, or a pharmaceutically acceptable salt thereof, wherein the compound contains a therapeutic radioisotope. In some embodiments, the method for treating cancer in a mammal comprises administering to the mammal a pharmaceutical composition comprising a compound of Formula IV, or a pharmaceutically acceptable salt thereof, wherein the compound contains a therapeutic radioisotope.

Examples of cancers that are treatable using the compounds of the present disclosure include, but are not limited to, sarcomas, bone cancer, breast cancer, lung cancer (e.g., non-small cell lung cancer and small cell lung cancer), genitourinary tract cancers, pancreatic cancer, liver cancers, skin cancers, melanoma (e.g., metastatic malignant melanoma, BRAF and HSP90 inhibition-resistant melanoma), cancer of the head or neck, cutaneous or intraocular malignant melanoma, uterine cancer, ovarian cancer, rectal cancer, cancer of the anal region, stomach cancer, renal cancer (e.g., clear cell carcinoma), prostate cancer (e.g., hormone refractory prostate adenocarcinoma), testicular cancer, colon cancer, gynecological cancers, uterine cancer, carcinoma of the fallopian tubes, urothelial cancer (e.g., bladder), carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, nervous system cancers, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including follicular lymphoma, including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

Exemplary hematological cancers include lymphomas and leukemias such as acute lymphoblastic leukemia (ALL), acute myelogenous leukemia (AMIL), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors.

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

In some embodiments, the methods of the present disclosure are used to detect, treat, or detect and treat, cancers exhibiting high fibrin levels.

In some embodiments of the methods of treatment of the present disclosure, the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, wherein the compound or pharmaceutical composition contains a therapeutic radioisotope, the method further comprises administering to the patient an amino acid solution. In some embodiments, the amino acid solution is used to prevent nephrotoxicity associated with radionuclide therapy. In some embodiments, the amino acid solution comprises L-lysine and L-arginine, and pharmaceutically acceptable salts and combinations thereof. In some embodiments, the amino acid solution comprises L-lysine and pharmaceutically acceptable salts thereof. In some embodiments, the amino acid solution comprises L-arginine and pharmaceutically acceptable salts thereof. In some embodiments, the amino acid solution comprises about 10 g/L to about 40 g/L of a mixture of L-lysine HCl and L-arginine HCl, such as about 12 g/L to about 35 g/L, about 16 g/L to about 32 g/L, about 20 g/L to about 30 g/L, or about 22 g/L to about 28 g/L. In some embodiments, the amino acid solution comprises about 16 g/L to about 32 g/L of a mixture of L-lysine HCl and L-arginine HCl. In some embodiments, the amino acid solution comprises between about 8 g/L and about 16 g/L of L-lysine HCl and between about 8 g/L and about 16 g/L of L-arginine HCl. In some embodiments, the amino acid solution comprises about 8 g/L, about 9 g/L, about 10 g/L, about 11 g/L, about 12 g/L, about 13 g/L, about 14 g/L, about 15 g/L, or about 16 g/L of L-lysine HCl. In some embodiments, the amino acid solution comprises about 8 g/L, about 9 g/L, about 10 g/L, about 11 g/L, about 12 g/L, about 13 g/L, about 14 g/L, about 15 g/L, or about 16 g/L of L-arginine HCl.

In some embodiments, the amino acid solution is administered intravenously. In some embodiments, the amino acid solution is administered prior to, concomitantly, after, or combinations thereof, administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, wherein the compound or pharmaceutical composition contains a therapeutic radioisotope.

In some embodiments, the amino acid solution is administered prior to administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof. In some embodiments, the amino acid solution is administered about 5 minutes to about 60 minutes prior to administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, such as about 10 minutes, about 15 minutes, about 20 minutes, about 25 minutes, about 30 minutes, about 35 minutes, about 40 minutes, about 45 minutes, about 50 minutes, about 55 minutes, or about 60 minutes prior. In some embodiments, the amino acid solution is administered about 30 minutes prior to administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof.

In some embodiments, the amino acid solution is administered concomitantly with the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof. In some embodiments, the amino acid solution is co-infused with the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof.

In some embodiments, the amino acid solution is administered after administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof. In some embodiments, the amino acid solution is administered immediately after or about 5 minutes to about 60 minutes after administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, such as about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes after. In some embodiments, the amino acid solution is administered about 30 minutes after administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof.

In some embodiments, the amino acid solution is administered prior to, concomitantly with, and after administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, wherein the compound or pharmaceutical composition contains a therapeutic radioisotope.

In some embodiments, the amino acid solution is administered prior to, and concomitantly with administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, wherein the compound or pharmaceutical composition contains a therapeutic radioisotope.

In some embodiments, the amino acid solution is administered prior to and after administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, wherein the compound or pharmaceutical composition contains a therapeutic radioisotope.

In some embodiments, the amino acid solution is administered concomitantly with and after administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, wherein the compound or pharmaceutical composition contains a therapeutic radioisotope.

In some embodiments of the methods of treatment of the present disclosure, the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, wherein the compound or pharmaceutical composition contains a therapeutic radioisotope, the method further comprises administering to the patient an antiemetic agent. An antiemetic agent can be used to reduce nausea and vomiting associated with radiotherapy. In some embodiments, the antiemetic agent is selected from the group consisting of 5-HT₃ receptor antagonists, corticosteroids, neurokinin-1 (NK-1) receptor inhibitors, prochlorperazine, metoclorpramide and cannabinoids. In some embodiments, the antiemetic agent is a 5-HT₃ receptor antagonist. In some embodiments, the antiemetic agent is a NK-1 receptor inhibitor.

In some embodiments, the antiemetic agent is administered prior to, concomitantly, after, or combinations thereof, administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, wherein the compound or pharmaceutical composition contains a therapeutic radioisotope. In some embodiments, the antiemetic agent is administered prior to administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof. In some embodiments, the antiemetic agent is administered concomitantly with the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof. In some embodiments, the antiemetic agent is administered after administering the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof.

In some embodiments, the antiemetic agent is administered prior to, concomitantly, after, or combinations thereof, administering an amino acid solution, such as the amino acid solutions described in the present disclosure. In some embodiments, the antiemetic agent is administered prior to administering the amino acid solution. In some embodiments, the antiemetic agent is administered concomitantly with the amino acid solution. In some embodiments, the antiemetic agent is administered after administering the amino acid solution. Also provided in the present disclosure are methods of detecting and treating a disease or condition associated with the presence of fibrin in a mammal. In some embodiments, the method comprises detecting the disease or condition associated with the presence of fibrin in a mammal using the methods described in the present disclosure that use the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, wherein the compound or pharmaceutical composition contains a radioisotope capable of detection using a nuclear imaging technique. In some embodiments, the method further comprises treating the disease or condition associated with the presence of fibrin in a mammal using the methods described in the present disclosure that use the compound of Formula I, Formula II, Formula III, or Formula IV, or a pharmaceutically acceptable salt thereof, or a pharmaceutical composition containing an effective amount of a compound of Formula I, Formula II, Formula III, or Formula IV, or a pharmaceutically acceptable salt thereof, wherein the compound or pharmaceutical composition contains a therapeutic radioisotope. In some embodiments, the disease or condition associated with the presence of fibrin is cancer.

### EXAMPLES

The invention is further described in the following examples, which do not limit the scope of the invention described in the claims.

### Example 1: Compound Synthesis and Characterization

### Step A—Synthesis of Unmodified Peptides

Peptides were synthesized on an automated peptide synthesizer “Liberty Blue” (CEM Inc.) using 1 to 12 batch reactors loaded with 0.1 mmol of commercially available Rink amide resin (~0.38 mmol/g). Standard Fmoc chemistry was used to elongate the peptide on the resin. The Fmoc was removed with a solution of 20% piperidine and 0.1 M HOBt in DMF. Each amino acid was dissolved in DMF to give a 0.2 M solution and was coupled to the peptide using a 0.5 M solution of diisopropylcarbodiimide in DMF, and 1.0 M Oxyma (or HOBt).

After the synthesis of the peptide on the resin was complete, the peptide was filtered and subsequently cleaved from the resin (TFA/TIS/MSA/2,2-(ethylenedioxy)ethane dithiol/H₂O 95:2.5:2.5:2.5:2.5). The solution of fully deprotected peptide was precipitated with diethyl ether (40 mL). The peptide solid was isolated after centrifugation and then cyclized in a 1:1 mixture of DMSO/40 mM NH₄OAc, pH 5. The cyclization was monitored by LC-MS (24h). The cyclic peptide was purified by reversed phase preparative HPLC on a C-5 column using a gradient of 5% mobile phase A (0.1% TFA in water) to 60% mobile phase B (0.1% TFA in acetonitrile) over 23 minutes. The fractions of pure peptide were pooled and lyophilized to give the final peptide moiety.

### Step B—Synthesis of N-Terminal Modified Peptides

The N-terminus of each peptide was then modified to:

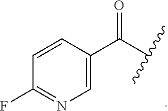 [SMILES: *C(=O)c1ccc(F)nc1]

See Tables 1 and 1a for the compounds (e.g., N-terminal modified peptides) prepared, where F is fluorine (Table 1) or fluorine-18 (Table 1a). All compounds possessed a C-terminal amide unless otherwise noted.

TABLE 1

| | | | SEQ | |
|---|---|---|---|---|
| Compound | N-terminus | Sequence | ID NO. | MW |
| 2 | 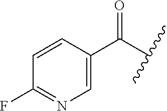 [SMILES: *C(=O)c1ccc(F)nc1] | -y-e-C-Hyp-Y(3-Cl)-G-L-C-H-I-Q- | 2 | 1496.05 |
| 3 | 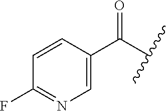 [SMILES: *C(=O)c1ccc(F)nc1] | -y-e-C-Hyp-Y(3-Cl)-G-L-C-H-I-1- | 3 | 1481.08 |
| 4 | 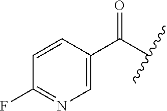 [SMILES: *C(=O)c1ccc(F)nc1] | -Y-e-C-Hyp-Y(3-Cl)-G-L-C-H-I-Q- | 4 | 1496.05 |
| 5 | 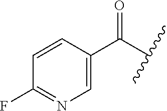 [SMILES: *C(=O)c1ccc(F)nc1] | -Y-e-C-Hyp-Y(3-Cl)-G-L-C-H-I-q- | 5 | 1496.05 |
| 6 | 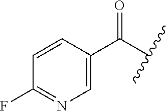 [SMILES: *C(=O)c1ccc(F)nc1] | -y-e-C-Hyp-Y(3-Cl)-G-L-C-Y-I-Q- | 6 | 1522.08 |
| 7 | 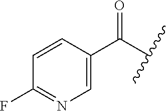 [SMILES: *C(=O)c1ccc(F)nc1] | -Y-e-C-Hyp-Y(3-Cl)-G-L-C-Y-I-Q- | 7 | 1522.08 |
| 8 | 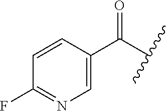 [SMILES: *C(=O)c1ccc(F)nc1] | -Y-e-C-Hyp-Y(3-Cl)-G-L-C-H-I-1- | 8 | 1481.08 |
| 9 | 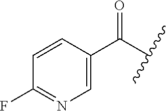 [SMILES: *C(=O)c1ccc(F)nc1] | -y-e-C-Hyp-Y(3-Cl)-G-L-C-Y-I-1- | 9 | 1507.11 |
| 10 | 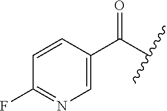 [SMILES: *C(=O)c1ccc(F)nc1] | -Y-e-C-Hyp-Y(3-Cl)-G-L-C-Y-I-1- | 10 | 1507.11 |
| 11 | 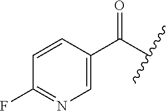 [SMILES: *C(=O)c1ccc(F)nc1] | -y-e-C-Hyp-Y(3-Cl)-G-L-C-Y-I-q- | 11 | 1522.08 |
| 12 | 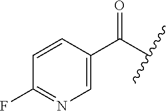 [SMILES: *C(=O)c1ccc(F)nc1] | -Y-e-C-Hyp-Y(3-Cl)-D-L-C-Y-I-Q- | 12 | 1580.12 |
| 13 | 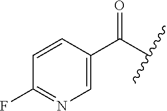 [SMILES: *C(=O)c1ccc(F)nc1] | -Y-e-C-Hyp-Y(3-Cl)-G-L-C-Y-I-q- | 13 | 1522.08 |
| 14 | 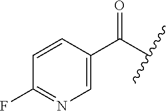 [SMILES: *C(=O)c1ccc(F)nc1] | -Y-e-C-Hyp-Y(3-Cl)-d-L-C-Y-I-Q- | 14 | 1580.12 |
| 15 | 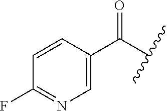 [SMILES: *C(=O)c1ccc(F)nc1] | -Y-h-C-Hyp-Y(3-Cl)-G-L-C-Y-I-Q- | 15 | 1530.11 |

TABLE 1a

| | | | SEQ | MW |
|---|---|---|---|---|
| Compound | N-terminus | Sequence | ID NO. | |
| 25 | 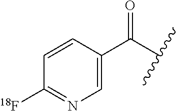 [SMILES: *C(=O)c1ccc([18F])nc1] | -y-e-C-Hyp-Y(3-Cl)-G-L-C-H-I-Q- | 27 | 1496.05 |
| 26 | 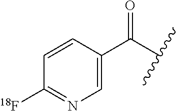 [SMILES: *C(=O)c1ccc([18F])nc1] | -y-e-C-Hyp-Y(3-Cl)-G-L-C-H-I-1- | 28 | 1481.08 |
| 27 | 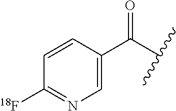 [SMILES: *C(=O)c1ccc([18F])nc1] | -Y-e-C-Hyp-Y(3-Cl)-G-L-C-H-I-Q- | 29 | 1496.05 |
| 28 | 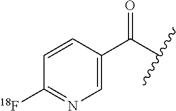 [SMILES: *C(=O)c1ccc([18F])nc1] | -Y-e-C-Hyp-Y(3-Cl)-G-L-C-H-I-q- | 30 | 1496.05 |
| 29 | 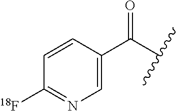 [SMILES: *C(=O)c1ccc([18F])nc1] | -y-e-C-Hyp-Y(3-Cl)-G-L-C-Y-I-Q- | 31 | 1522.08 |
| 30 | 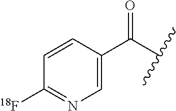 [SMILES: *C(=O)c1ccc([18F])nc1] | -Y-e-C-Hyp-Y(3-Cl)-G-L-C-Y-I-Q- | 32 | 1522.08 |
| 31 | 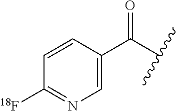 [SMILES: *C(=O)c1ccc([18F])nc1] | -Y-e-C-Hyp-Y(3-Cl)-G-L-C-H-I-1- | 33 | 1481.08 |
| 32 | 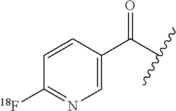 [SMILES: *C(=O)c1ccc([18F])nc1] | -y-e-C-Hyp-Y(3-Cl)-G-L-C-Y-I-1- | 34 | 1507.11 |
| 33 | 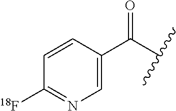 [SMILES: *C(=O)c1ccc([18F])nc1] | -Y-e-C-Hyp-Y(3-Cl)-G-L-C-Y-I-1- | 35 | 1507.11 |
| 34 | 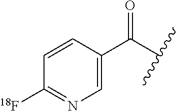 [SMILES: *C(=O)c1ccc([18F])nc1] | -y-e-C-Hyp-Y(3-Cl)-G-L-C-Y-I-q- | 36 | 1522.08 |
| 35 | 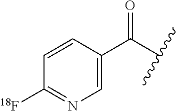 [SMILES: *C(=O)c1ccc([18F])nc1] | -Y-e-C-Hyp-Y(3-Cl)-D-L-C-Y-I-Q- | 37 | 1580.12 |
| 36 | 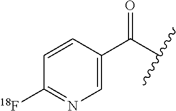 [SMILES: *C(=O)c1ccc([18F])nc1] | -Y-e-C-Hyp-Y(3-Cl)-G-L-C-Y-I-q- | 38 | 1522.08 |
| 37 | 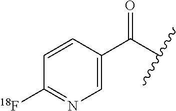 [SMILES: *C(=O)c1ccc([18F])nc1] | -Y-e-C-Hyp-Y(3-Cl)-d-L-C-Y-I-Q- | 39 | 1580.12 |
| 38 | 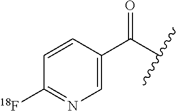 [SMILES: *C(=O)c1ccc([18F])nc1] | -Y-h-C-Hyp-Y(3-Cl)-G-L-C-Y-I-Q- | 40 | 1530.11 |

### Example 2: Synthesis of PFP-Peptides

Unmodified peptides were prepared according to the procedure in Example 1, step A. 6-Fluoronicotinic acid-PFP (0.10 mmol) was coupled to the N-terminus of the peptides (0.02 mmol) in DMF (0.50 mL) at 40° C. overnight. The reaction was monitored by LCMS. The obtained crude compound was purified by reversed phase preparative HPLC C-5 column (22.5×250 mm) using a gradient of 500 mobile phase A (0.1% TFA in water) to 60% mobile phase B (0.1% TFA in acetonitrile) over 23 min and pooled fractions were lyophilized to obtain the pure compound.

### Example 3: Determination of Binding to Immobilized Fibrin and Soluble Fibrin Fragment DD(E) Using a Fluorescence Polarization (FP) Assay

DD(E) was prepared using a published procedure which involved partial plasmin digestion of purified fibrin gel followed by size-exclusion chromatographic purification, and purity was assessed by SDS-PAGE. Peptides modified at the N-terminus with tetramethyl-rhodamine (TRITC) dye bind to DD(E). Direct fluorescent peptide binding to DD(E) was measured by a fluorescence polarization (FP) assay. The anisotropy (r_{obs}) of the fluorescently labeled peptides binding to DD(E) (0-20 μM) was measured in Tris buffered saline (50 mM Tris, 100 mM NaCl and 2 mM CaCl₂) at fixed compound concentrations, and the data were fit to a single site model (Eq. 1) to obtain the dissociation constant (K_{d}) for the DD(E)·(fluorescent peptide) complex.

$$\sqrt{\begin{matrix}{r}_{obs} = {r}_{fr} + \frac{{r}_{bd} - {r}_{fr}}{{[ Fl ]}_{T}} \times \frac{b - \sqrt{{b}^{2} + {{4 [ Fl ]}_{t} [ DD ( E ) ]}_{t}}}{2} ; \\ b = ( {[ DD ( E ) ]}_{t} + {[ Fl ]}_{t} + {K}_{d} )\end{matrix}} \qquad Eq . 1$$

Displacement of the fluorescent peptide by non-fluorescent compound was measured to determine inhibition constants (Kᵢ). In the presence of an inhibitor, an apparent dissociation constant for the fluorescent compound (K_{d} app) is determined using Eq. 2. The inhibition constant Kᵢ is related to K_{d} app by Eq. 2, wherein K_{d} is the true dissociation constant of the fluorescent compound measured in the absence of inhibitor.

$${K}_{d}^{app} = {K}_{d} \{ 1 + \frac{{[ Inhibitor ]}_{free}}{Ki} \} \qquad Eq . 2$$

Non-labeled peptide binding to DD(E) was measured by a compound displacement assay, where a DD(E)/Fl·pep complex formed at fixed concentrations of DD(E) and Fl·pep was titrated with increasing concentrations of unlabeled compound. The compound was assayed by mixing DD(E) (4.0 μM, 10 μL) and TRITC (0.1 μM, 10 μL) with increasing concentrations of competing peptide (0.4-100 μM, 20 μL) in TBS·Ca with 2% DMSO in a final volume of 40 μL. Fluorescence polarization of the samples (10 μL, triplicate wells) in ascending concentrations was measured in a 384-well microplate (Corning flat black plate), using a Tecan Infinite 200 Pro fluorescence microplate reader equipped with fluorescence polarization filters, a 535 nm excitation filter and a 590 nm fluorescence emission filter. Average polarization (mP units) readings for each dilution of the compound of interest was converted to anisotropy (r) using Eq. 3.

$$\sqrt{{r}_{obs} = \frac{( 0.002 \times mP )}{( 3 - 0. \times mP )}} \qquad Eq . 3$$

The data defined a displacement curve (r_{obs} vs. [Peptide]), and the inhibitory binding constant, Kᵢ, was determined by least squares fitting of the data (estimated uncertainty in Kᵢ±10%). See Table 2 for the Kᵢ values for the Compounds from Example 1.

TABLE 2

| Compound | Kᵢ (μM) |
|---|---|
| 2 | 0.07 |
| 3 | 0.14 |
| 4 | 0.18 |
| 5 | 0.23 |
| 6 | 0.34 |
| 7 | 0.58 |
| 8 | 0.64 |
| 9 | 0.73 |
| 10 | 0.95 |
| 11 | 1.01 |
| 12 | 1.2 |
| 13 | 1.25 |
| 14 | 2.76 |
| 15 | No |
| | displacement |

### Results

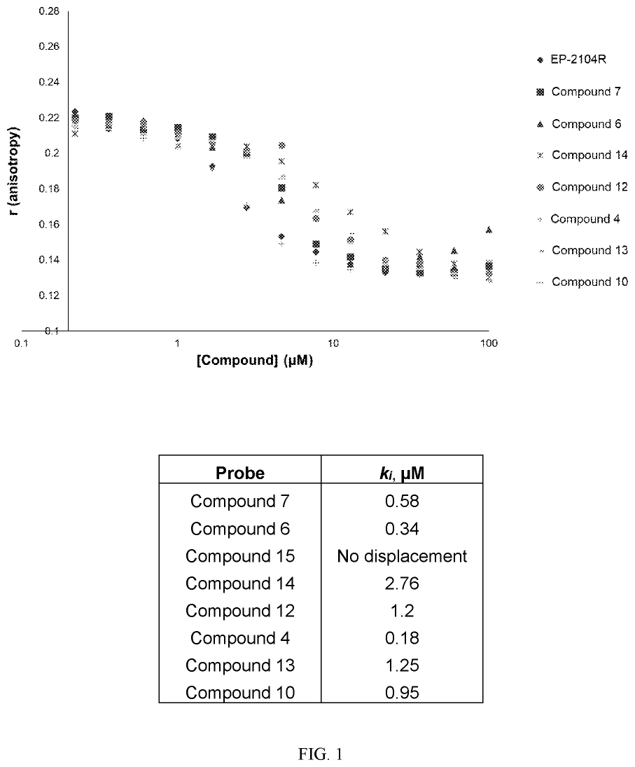

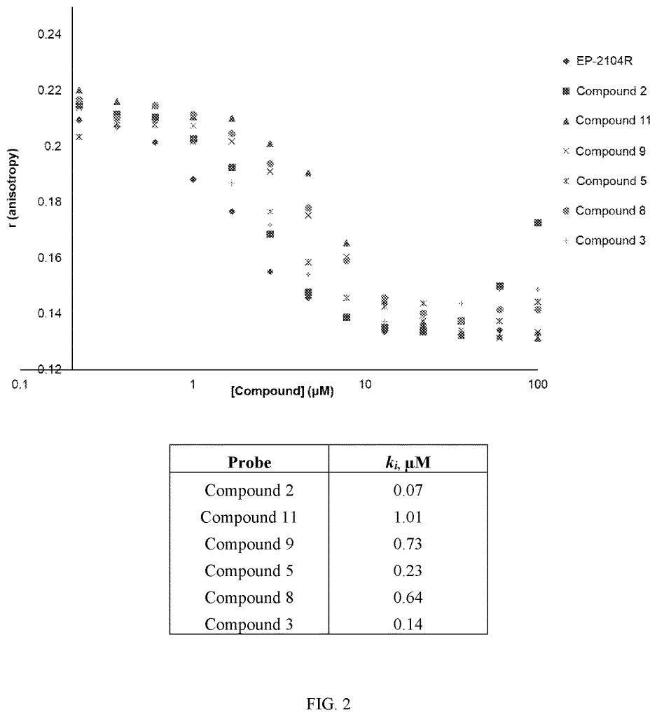

Affinity to soluble fibrin fragment DD(E) was screened for all the compounds from series A and C using fluorescent polarization. Compounds were compared to EP-2104R, a reference compound with validated fibrin affinity (FIG. 1 and FIG. 2). Among the 14 compounds tested, six (Compounds 7, 6, 4, 2, 5, and 3) were identified as having sub-micromolar affinity (K_{d}<0.6 μM) to fibrin as assessed by DD(E) assay.

### Example 4: Determination of Metabolic Stability Using a Rat Serum Assay

The six compounds identified in Example 3 (Compounds 2, 3, 4, 5, 6, and 7) were assayed for stability in rat plasma at 37° C. for up to 4 h. Compounds were dissolved in DMSO to a make a stock solution (0.6 mM). Plasma (995 μL) was spiked with peptide stock solution (5 μL). Incubations were carried at a test compound concentration of 3 μM with a final DMSO concentration of 2.5%. The spiked plasma samples were incubated at 37° C. for 4 h. Aliquots (300 μL) were removed at 0, 0.5, 1, 2 and 4 h and reactions were terminated by adding 600 μL of cold acetonitrile with 0.1% formic acid containing carbutamide (25 μg/mL) as an internal standard. Simultaneously, a plasma sample (995 μL) containing a mixture of benfluorex, propranolol and nortriptyline (40 μM in DMSO) control compounds was also incubated alongside each batch of test compounds and terminated similarly. In addition, matrix blanks were prepared by adding acetonitrile-containing internal standard to plasma samples without any of the analytes or control compounds. Aliquots were centrifuged at 5000×g for 5 min for protein precipitation. Following centrifugation, the concentration of the compound of interest in the supernatant was quantified by LCMS. The presence of peptides was confirmed by detection of the corresponding [(M+2)/2]⁺. The peak area for each detected compound was measured to determine the % remaining of each compound and the result was compared with standards of pure compound and compound immediately isolated from plasma (t=0 sample). All assay samples were analyzed in triplicate. LCMS measurements were performed with a gradient elution system (5-95%) composed of water and acetonitrile with 0.1% TFA at a flow rate of 1 mL/min using a Phenomenex-C18 chromatographic column (3 μm particle size, 4.6 mm×100 mm).

### Results

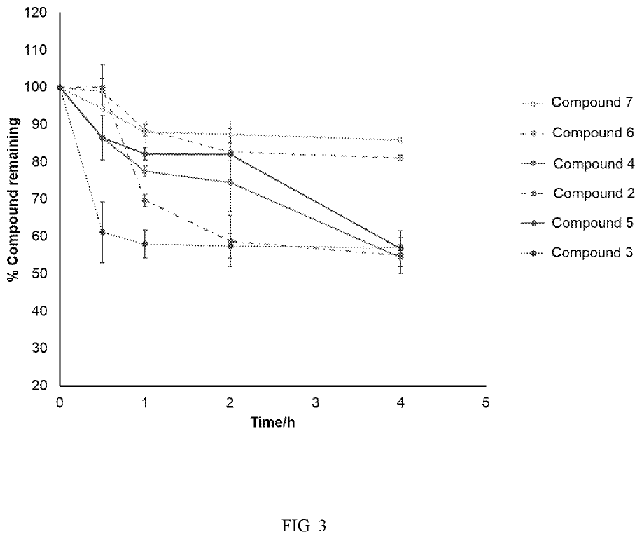

Three compounds were more than 80% intact at the 2 h time point, with Compounds 7 and 6 demonstrating the greatest stability after 4 h (FIG. 3).

### Example 5: Development of F-18 Radiolabeling Conditions and Analytical Methods for the Optimized Compounds

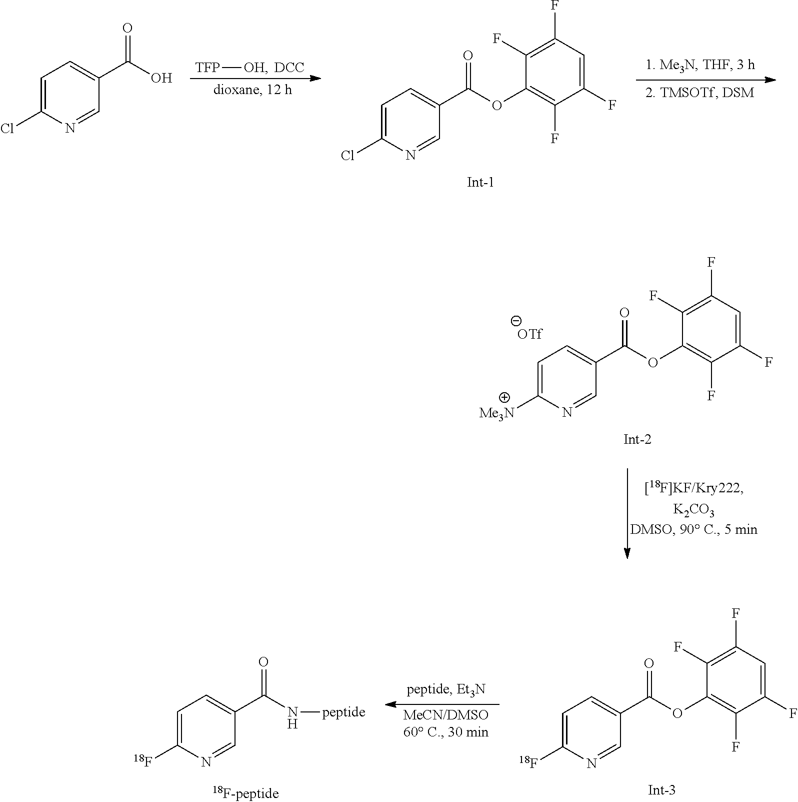

### Synthesis of 6-chloronicotinic Acid 2,3,5,6-Tetrafluorophenyl ester (Int-1)

6-Cl-Py-PFP (Int-1) ester was obtained by the reaction of 6-chloronicotinic acid (1.0 g, 7.1 mmol), tetrafluorophenol (TFP, 1.18 g, 7.1 mmol), in the presence of N,N′-dicyclohexylcarbodiimide (DCC) (1.40 g, 6.81 mmol) in dioxane (35 mL) at room temperature for 12 h. Dicyclohexylurea (DCU) was filtered off and the solvents were removed to obtain the crude product which was crystalized from hot hexane (1.61 g, 85%). Compound identity was confirmed by ¹H NMR analysis.

### Synthesis of N,N,N-trimethyl-5-((2,3,5,6-tetrafluorophenoxy)-carbonyl)pyridin-2-aminium trifluoromethanesulfonate (Int-2)

A steady stream of trimethylamine gas was passed through a mixture of Int-1 (1.0 g, 3.3 mmol) in dry THF (15 mL) at room temperature for 3 h. The obtained N,N,N-trimethyl-5-((2,3,5,6-tetrafluoro-phenoxy)carbonyl)pyridin-2-aminium chloride was filtered then washed with diethyl ether (100 mL) and cold dichloromethane (50 mL). The solid (0.53 g, 1.5 mmol) was suspended in DCM (50 mL) under an argon atmosphere. The solution was filtered, and volatile components were removed under reduced pressure. The residue was washed with diethyl ether (3×50 mL) and dried under vacuum to afford Int-2 (0.65 g, 41.6%). Compound identity was confirmed by ¹H NMR analysis.

Radiochemical Synthesis of ¹⁸F-Py-TFP and Compound ¹⁸F-7

[¹⁸F] Fluoride was trapped in Chromafix PS-HCO₃⁻ cartridge pretreated with 1 ml of 1 M potassium carbonate solution and 20 mL of water by passing a [¹⁸O] water containing [¹⁸F]fluoride through the cartridge. [¹⁸F]Fluoride was eluted from the cartridge using a 0.4 ml of potassium carbonate and KRYPTOFIX® 222 (Kry222) in acetonitrile:water 1:1 solution. [¹⁸F]Fluoride was dried by azeotropic evaporation at 105° C. in a stream of nitrogen by adding acetonitrile during evaporation (0.5 mL×3 times). After it was dried completely, a DMSO solution (0.3 mL) containing Int-2 (0.6 mg, 12.54 μmol) was added to the residue and heated at 90° C. for 5 min. The reaction solution was diluted with acetonitrile:0.1% TFA solution=1:1 co-solvent and injected into Alltima C₁₈ Semi-Preparative Column (250 mm×10 mm, 5 μm) eluted with 0.1% TFA in acetonitrile:0.1% TFA in water=64:36 at a flow rate of 4 mL/min. ¹⁸F-PFP ester was eluted between 13.0-13.5 min and it was collected in a C₁₈ Sep-Pak Plus Cartridge. ¹⁸F-PFP ester was released from the Sep-Pak using 0.9 ml of acetonitrile. DMSO (0.9 ml) containing TEA and peptide (0.5 mg, 0.36 μmol) was added to the reaction vessel. The mixture was heated at 60° C. for 30 min. The reaction was terminated by diluting with 0.9 mL of water. The whole aliquot was injected into Gemini-NX C₁₈ Semi-Preparative Column (250 mm×10 mm, 5 μm) eluted with 0.1% TFA in acetonitrile:0.1% TFA in water=33:67 at a flow rate of 4 mL/min. Compound ¹⁸F-7 was eluted between 12.0-12.5 min and it was collected in C₁₈ Sep-Pak Plus Cartridge. The product was obtained using 1 mL of ethanol.

### Results:

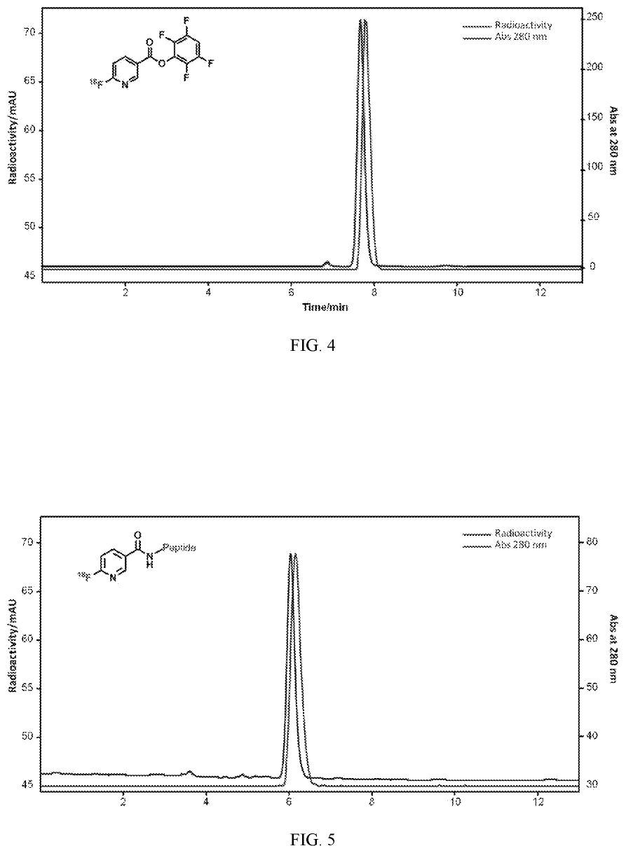

F-18 radiolabeling conditions were developed along with and analytical methods for the optimized compounds. FIG. 4 and FIG. 5 depict the radio-HPLC traces (red) of the 2-step radiochemical synthesis, namely preparation of ¹⁸F-Py-TFP (FIG. 4) and Compound ¹⁸F-7 (FIG. 5). The blue trace represents the UV detection traces of non-radioactive pure compounds. The radioactivity detector is positioned after the UV detector and so there is an offset in retention times between the UV trace and the radioactivity trace.

### Example 6: Evaluation of Specificity by Competitive Binding with Fibrinogen and Plasma Proteins

A plate-based assay was developed to directly measure compound affinity to fibrin. The fibrin is immobilized and competition from soluble proteins is measured. Compounds was assayed for fibrin binding in the presence or absence of human plasma.

### Fibrin Plate Preparation

Human fibrinogen (1 g) was dissolved in 30 mL of TBS buffer (50 mM Tris, 150 mM NaCl, 5 mM sodium citrate, pH 7.4) and dialyzed in a Slyde-a-Lyzer (20 000 MWCO, Cassette G2) at room temperature. After two changes of buffer, the fibrinogen was centrifuged (10 min, 2000×g) to remove undissolved material. Fibrinogen concentration was determined by measuring the absorbance at 280 nm. Stock fibrinogen solution concentration was 32.1 mg/mL. Fibrin plates with alternating rows of clotted fibrin and empty wells were prepared by polymerizing fibrinogen (100 μL; 2.5 mg/mL) with thrombin (1 U/mL) in TBS·citrate supplemented with 7 mM CaCl₂ in the wells of a 96 well polystyrene microtiter plate (Immulon-II®). The uncovered plates were dried overnight at 37° C. to afford a thin film which was adsorbed to the plate, sealed with tape, and stored at −20° C. until use. Clottable protein in individual fibrinogen batches was determined by measuring 280 nm absorbance of the soluble fraction of the solution before and after thrombin treatment, and was generally >96% (Fibrin concentration, 7.56 μM).

### F-18 Radioactivity Based Assay Protocol for Fibrin Affinity and Specificity

An aliquot (100 μL) from a known activity (52 μCi in 10 mL TBS buffer) of Compound ¹⁸F-7 was added to a series of 12 known concentrations (0.01-50 μM) of cold Compound 7 (550 μL) in TBS buffer and mixed well to prepare 12 stock solutions. Each dilution (100 μL) was added to both a clotted fibrin well and an empty well in duplicate. The same protocol was followed using human plasma instead of TBS buffer. Then the prepared plates were covered and incubated at room temperature for 2 h under constant agitation. An aliquot (90 μL) was carefully pipetted into a tared test tube, from the supernatant of each well. All aliquots from supernatants as well as an aliquot (90 μL) from each stock solution were weighed and activity of each was measured using a Cobra 5002 gamma counter. All data were decay corrected.

### Data Analysis

Using the data obtained from empty wells, corresponding fibrin clotted wells and the stock solutions, the [Compound 7]ₜₒₜₐₗ and [Compound 7]_{free} determined. [Compound 7]_{bound} for each sample was calculated as follows:

[Compound 7]_{bound}=[Compound 7]ₜₒₜₐₗ−[Compound 7]_{free}

Using [Compound 7]_{bound} and known [Fibrin]ₜₒₜₐₗ, [Compound 7]_{bound}/[Fibrin]ₜₒₜₐₗ was calculated. Active binding sites (N) and dissociation constant (K_{d}) were determined by plotting [Compound 7]_{bound}/[Fibrin]ₜₒₜₐₗ vs [Compound 7]_{free}. By the theoretical equation,

$$\frac{{[ Peptide ]}_{bound}}{{[ Fibrin ]}_{total}} = \frac{N \cdot {[ Peptide ]}_{free}}{( {[ Peptide ]}_{free} + {K}_{d} )}$$

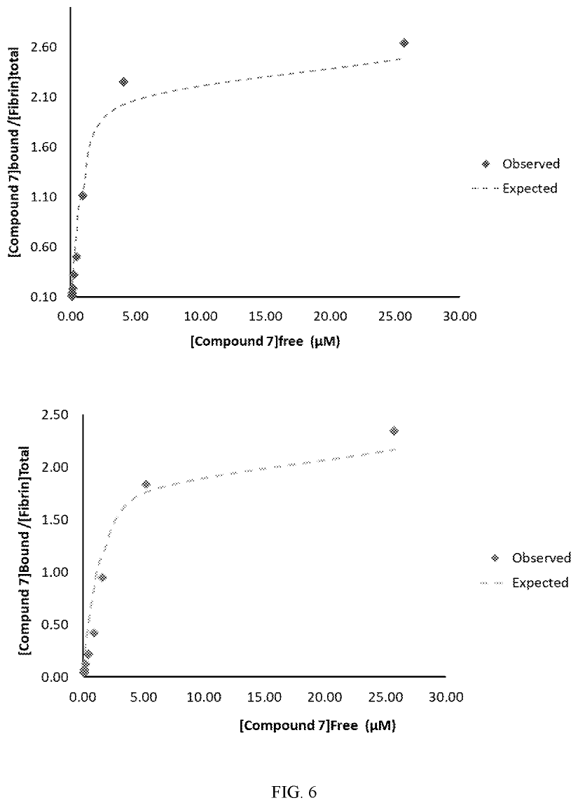

N and K_{d} variables were adjusted by solver to minimize absolute error between observed and theoretical values (FIG. 6).

### Compound ¹⁸F-7 Binding to Human Fibrin in TBS Buffer and in the Presence of Human Plasma

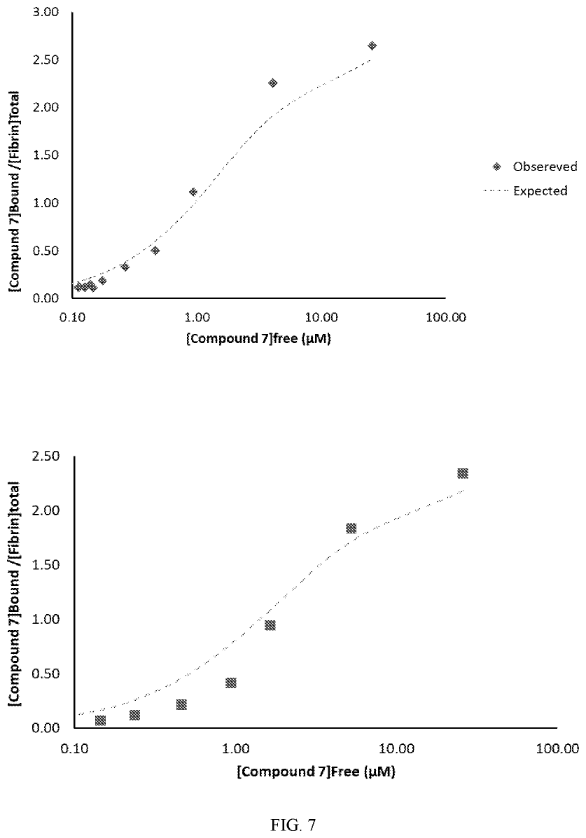

The similarities in the results obtained in the presence and absence of plasma proteins indicate that Compound ¹⁸F-7 has a high degree of binding specificity for fibrin over fibrinogen (FIG. 7).

### Example 7: Compounds with ⁶⁸Ga, ⁶⁴Cu, or Al¹⁸F Chelators

### NODAGA Chelators

Synthesis of (^{t}Bu)₃NODAGA-NHS ester: 4-(4,7-bis(2-(tert-butoxy)-2-oxoethyl)-1,4,7-triazacyclononan-1-yl)-5-(tert-butoxy)-5-oxopentanoic acid ((^{t}Bu)₃NODAGA-COOH, 100 mg, 0.19 mmol, 1.0 equiv.), N,N,N′,N′-tetramethyl-O-(1H-benzotriazol-1-yl)uronium hexafluorophosphate, O-(benzotriazol-1-yl)-N,N,N′,N′-tetramethyluronium hexa-fluorophosphate (HBTU, 122 mg, 0.3 mmol, 1.2 equiv.) and N-hydroxysuccinimide (NHS, 37.4 mg, 0.3 mmol, 1.2 equiv.) were dissolved in acetonitrile (10 mL) and stirred at room temperature for 24 h. After removal of the solvent under reduced pressure, the resulting residue was re-dissolved in dichloromethane (10 mL) and then promptly washed with water (3×4 mL). The organic layer was dried over magnesium sulfate, filtered and evaporated to afford the title product as a white foam (141 mg, 0.22 mmol, 85%).

Syntheses of (^{t}Bu)₃NODAGA-peptides: (^{t}Bu)₃NODAGA-NHS (1.5 equiv.) was added to a solution of each peptide (1 equiv.) in dimethylformamide (1 mL). The pH of each solution was adjusted to 6.5 using diisopropylethylamine (DIPEA) and the mixtures were stirred at room temperature for 24 hours. The reaction was monitored by LCMS. Upon completion of the reaction, the (^{t}Bu)₃NODAGA-peptide was purified by reversed phase preparative HPLC on a C-5 column (Luna, 10μ, 250×21.2 mm) using a gradient of 5% mobile phase A (0.1% TFA in water) to 60% mobile phase B (0.1% TFA in acetonitrile) over 45 minutes. Purified compound was dried under lyophilization to afford the title product.

Synthesis of Compound 16, Compound 17, Compound 18, Compound 19, Compound 20, and Compound 21: In a reaction vessels, about 10 mg of the (^{t}Bu)₃NODAGA-peptide were deprotected in a 1 mL solution of TFA, methanesulfonic acid, 1-dodecanethiol and H₂O (92:3:3:2). Each reaction mixture was stirred for 2 h and analyzed by LCMS. Upon completion of the reaction, cold diethyl ether (15 mL) was added to precipitate out the solids. The mixture was centrifuged, and the supernatant was removed. The solids were washed with diethyl ether and dried to give the product as white solids.
Compound 16: C₇₇H₁₀₇ClN₁₆O₂₅S₂, MS(ESI) calc: 878.3 [(M+2H)/2]²⁺; found: 878.4.
Compound 17: C₇₇H₁₀₇ClN₁₆O₂₅S₂, MS(ESI) calc: 878.3 [(M+2H)/2]²⁺; found: 878.5.
Compound 18: C₇₄H₁₀₅ClN₁₈O₂₄S₂, MS(ESI) calc: 866.2 [(M+2H)/2]²⁺; found: 866.1.
Compound 19: C₇₄H₁₀₅ClN₁₈O₂₄S₂, MS(ESI) calc: 866.1 [(M+2H)/2]²⁺; found: 866.3.
Compound 20: C₇₄H₁₀₅ClN₁₈O₂₄S₂, MS(ESI) calc: 866.1 [(M+2H)/2]²⁺; found: 866.1.
Compound 21: C₇₅H₁₀₈ClN₁₇O₂₃S₂, MS(ESI) calc: 857.9 [(M+2H)/2]²⁺; found: 858.7
Compound 22: C₇₄H₁₀₅ClN₁₈O₂₄S₂, MS(ESI) calc: 866.3 [(M+2H)/2]²⁺; found: 866.1.

TABLE 3
NODAGA-modified peptides

| | | SEQ ID |
|---|---|---|
| Compound | Sequence | NO. |
| 16 | NODAGA-Y-e-C-Hyp-Y(3-Cl)-G-L-C-Y-I-Q- | 18 |
| | NH₂ | |
| 17 | NODAGA-y-e-C-Hyp-Y(3-Cl)-G-L-C-Y-I-Q- | 19 |
| | NH₂ | |
| 18 | NODAGA-Y-e-C-Hyp-Y(3-Cl)-G-L-C-H-I-Q- | 20 |
| | NH₂ | |
| 19 | NODAGA-y-e-C-Hyp-Y(3-Cl)-G-L-C-H-I-Q- | 21 |
| | NH₂ | |
| 20 | NODAGA-Y-e-C-Hyp-Y(3-Cl)-G-L-C-H-I-q- | 22 |
| | NH₂ | |
| 21 | NODAGA-y-e-C-Hyp-Y(3-Cl)-G-L-C-H-I-I-NH₂ | 23 |
| 22 | NODAGA-y-e-c-Hyp-Y(3-Cl)-G-L-C-H-I-q-NH₂ | 24 |

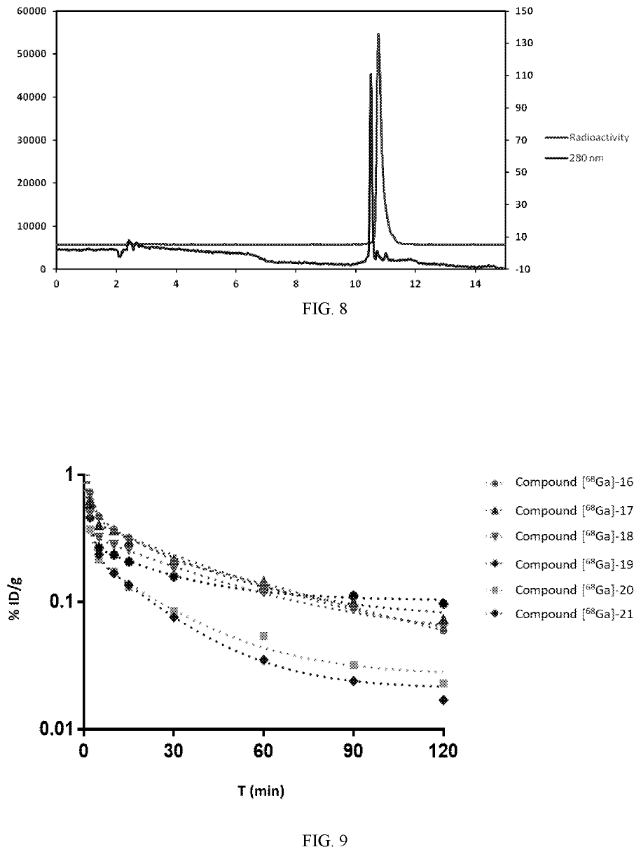

Radiochemical synthesis of Compounds ⁶⁸Ga-16, ⁶⁸Ga-17, ⁶⁸Ga-18, ⁶⁸Ga-19, ⁶⁸Ga-20, and ⁶⁸Ga-21, and ⁶⁸Ga-22: ⁶⁸GaCl₃ (1 mCi, in 0.5 mL HCl (0.6 M)) was diluted with of pH 5, 3M sodium acetate (200 μL) to reach pH 4.1. ⁶⁸GaCl₃ solution (200 μL) was combined with each NODAGA-peptide (0.1 mM 10 μL) solution in sodium acetate (10 mM, pH 4.1) and the reaction mixture was heated at 60° C. for 10 min and purified by Sep-Pak light C₁₈ cartridge (Waters) to remove any radiometal impurities (germanium-68 breakthrough). The radiochemical purity of the final solutions of Compound ⁶⁸Ga-16, Compound ⁶⁸Ga-17, Compound ⁶⁸Ga-18, Compound ⁶⁸Ga-19, Compound ⁶⁸Ga-20 (FIG. 8), Compound ⁶⁸Ga-21 and Compound ⁶⁸Ga-22 was >95% as determined by radio-HPLC analysis.

Radiochemical synthesis of Compound 64Cu-20: ⁶⁴CuCl₂ (1 mCi), in 0.5 mL HCl (0.6 M) was diluted with 3M sodium acetate (200 μL) to reach pH 4.5. A solution of NODAGA-Peptide 22 (0.1 mM 10 μL) in sodium acetate (10 mM, pH 4.5) was added heated at 60° C. for 10 min. The radiochemical purity of Compound 64Cu-20 was ≥95% as determined by radio-HPLC analyses.

### DOTAGA Chelators

Synthesis of (^{t}Bu)₄DOTAGA-PFP: (^{t}Bu)₄DOTAGA (200 mg, 0.29 mmol) and pentafluorophenol (88 mg, 0.48 mmol) were dissolved in dichloromethane (1 mL) and PS-DCC (286 mg, 0.48 mmol, 1.67 mmol/g) was added to the mixture. The reaction was stirred at room temperature and monitored by HPLC. Upon completion of the reaction, the resin was removed by filtration. The filtrates were evaporated, and the residue was dried under reduced pressure. The obtained crude (^{t}Bu)₄DOTAGA-PFP was used in next step without further purification. (Theoretical MW for [M+H]⁺=868.0, observed 867.7).

Synthesis of (^{t}Bu)₄DOTAGA-peptides: (^{t}Bu)₄DOTAGA-PFP (50 mg, 0.06 mmol) and the peptide (79.2 mg, 0.06 mmol) were dissolved in DMF (1 mL) and pH was maintained around 6.5 by adding DIPEA. The reaction was monitored by HPLC. Upon completion of the reaction, solids were precipitated with brine, washed with distilled water and dried under reduced pressure. The crude product was used in the next step without further purification. (Theoretical MW for [M+H]⁺=2056.9, observed 2056.7).

Synthesis of Compound 23: The t-butyl esters were cleaved by stirring the (^{t}Bu)₄DOTAGA-peptide in a mixture of TFA, triisopropylsilane, dodecanethiol, MSA and water (92:2:2:2:2, 1 mL) at room temperature overnight. The reaction was monitored by HPLC. The reaction mixture was precipitated in cold diethylether. The solid was filtered, washed with cold diethylether and dried under reduced pressure. The crude product was dissolved in water (6 mL) and purified by reversed phase preparative HPLC on a C-18 column (Luna, 10μ, 250×21.2 mm) using a gradient of 5% mobile phase A (0.1% TFA in water) to 95% mobile phase B (0.1% TFA in acetonitrile) over 30 minutes. The fractions of pure Compound 23 were combined and lyophilized to obtain a white powder.

Compound 23: C₇₈H₁₁₂ClN₁₉O₂₆S₂, MS(ESI) calc: 1832.43 [M+H]⁺, 916.7 [(M+2H)/2]²⁺; found: 1831.7 [M+H]⁺, 916.2 [(M+2H)/2]²⁺.

TABLE 5
DOTAGA-modified peptide

| | | SEQ ID |
|---|---|---|
| Compound | Sequence | NO. |
| 23 | DOTAGA-Y-e-C-Hyp-Y(3-Cl)-G-L-C- | 25 |
| | H-I-q-NH₂ | |

Radiochemical synthesis of Compound ⁶⁸Ga-23: A SCX cartridge (100 mg, particle size m (Agilent, cat. no. 12102013)) was preconditioned first by washing with 5.5 M HCl (1 mL) and then by washing with water (10 mL). Ga-68 (3.0 mCi) was eluted with 4 mL of 0.05 M HCl and loaded on a preconditioned SCX cartridge. The cartridge was purged with air and ⁶⁸GaCl₃ was eluted with 0.3 mL of 3M NaCl solution (containing 0.1 M HCl). 0.15 mL of ⁶⁸GaCl₃ was added to the solution of Compound 23 (25 μL, 1.0 mM) mixed with 0.15 mL of 1.5M NaOAc buffer. The reaction mixture was heated at 90° C. for 10 min and analyzed by radio-HPLC. The radiochemical purity was ≥99% as determined by radio-HPLC analysis.

Radiochemical synthesis of Compound ⁹⁰Y-23: A 100 μL solution containing 0.5 mCi of ⁹⁰YCl₃ is added to the solution of Compound 23 (25 μL, 1.0 mM) and is mixed with 0.15 mL of pH 5 sodium acetate buffer. The reaction mixture is heated at 40° C. for 60 min and analyzed by radio-HPLC. with gamma counting of HPLC fractions.

Radiochemical synthesis of Compound ¹⁷⁷Lu-23: A 100 μL solution containing 0.5 mCi of ¹⁷⁷LuCl₃ is added to the solution of Compound 23 (25 μL, 1.0 mM) and is mixed with 0.15 mL of pH 5 sodium acetate buffer. The reaction mixture is heated at 40° C. for 60 min and analyzed by radio-HPLC.

Radiochemical synthesis of Compound ²²⁵Ac-23: A 100 μL solution containing 0.1 mCi of ²²⁵Ac(NO₃)₃ and 20% L-ascorbic acid is added to the solution of Compound 23 (25 μL, 1.0 mM) and is mixed with 0.15 mL of pH 6 Tris buffer. The reaction mixture is heated at 60° C. for 60 min and analyzed by radio-HPLC with gamma counting of HPLC fractions.

### NOTA Chelators

Syntheses of (^{t}Bu)₂NOTA-peptide: NOTA(^{t}Bu)₂ (50 mg, 0.12 mmol) and HATU (68.6 mg, 0.18 mmol) were dissolved in DMF (1 mL) and the mixture was stirred at room temperature for 30 min. The peptide (181 mg, 0.13 mmol) was dissolved in DMF (1 mL) and was added and the pH was maintained at 6.5 with DIEA. The reaction was monitored by LC-MS. Upon completion of the reaction, the mixture was diluted with water (4 mL) and purified by reversed phase preparative HPLC on a C-18 column (Luna, 10μ, 250×21.2 mm) using a gradient of 5% mobile phase A (0.1% TFA in water) to 95% mobile phase B (0.1% TFA in acetonitrile) over 30 minutes. The fractions of pure compound were pooled and lyophilized to obtain the final product. (Theoretical MW for [M+H]⁺=1771.5, observed 1771.7).

Synthesis of Compound 24: In a reaction vessel, about 35 mg of the (^{t}Bu)₂NOTA-peptide was deprotected in a 1 mL solution of TFA, methanesulfonic acid, 1-dodecanethiol, triisopropylsilane and H₂O (92:2:2:2:2). The reaction mixture was stirred for 2 h at room temperature, and analyzed by LCMS. Upon completion of the reaction, cold diethyl ether (15 mL) was added to precipitate out the solids. The mixture was centrifuged, and the supernatant was removed. The solids were washed with diethyl ether and dried to give the product as a white solid. The crude product was dissolved in water (5 mL) and purified by reversed phase preparative HPLC on a C-18 column (Luna,10μ, 250×21.2 mm) using a gradient of 5% mobile phase A (0.1% TFA in water) to 95% mobile phase B (0.1% TFA in acetonitrile) over 30 minutes. The fractions of pure Compound 24 were pooled and lyophilized to obtain a white powder..

Compound 24: C₇₁H₁₀₁ClN₁₈O₂₂S₂, MS(ESI) calc: 1659.3 [M+H]⁺, 830.1 [(M+2H)/2]²⁺; found: 1659.7 [M+H]⁺, 829.7 [(M+2H)/2]²⁺.

TABLE 4
NOTA-modified peptide

| | | SEQ ID |
|---|---|---|
| Compound | Sequence | NO. |
| 24 | NOTA-Y-e-C-Hyp-Y(3-Cl)-G-L-C- | 26 |
| | H-I-q-NH₂ | |

Radiochemical synthesis of Compound ⁶⁸Ga-24: A SCX cartridge (100 mg, particle size m (Agilent, cat. no. 12102013)) was preconditioned first by washing with 5.5 M HCl (1 mL) and then by washing with water (10 mL). Ga-68 (3.2 mCi) was eluted with 4 mL of 0.05 M HCl and loaded onto the preconditioned SCX cartridge. The cartridge was purged with air and ⁶⁸GaCl₃ was eluted with 0.3 mL of 3 M NaCl solution (containing 0.1 M HCl). ⁶⁸GaCl₃ (0.15 mL) was added to a solution of Compound 24 (25 μL, 0.6 mM) in NaOAc buffer (1.5 M, 0.15 mL, pH 4.5). The reaction mixture was heated at 90° C. for 10 min and analyzed by radio-HPLC. The radiochemical purity of the final product was >98%.

Radiochemical synthesis of Compound Al¹⁸F-24: A sep-Pak Light Accell Plus QMA cartridge (Waters) was pre-conditioned by passing 10 mL of 0.4 M KHCO₃ followed by 10 mL of DI water. ¹⁸F (2.0 mCi, 200 μL) was loaded onto the cartridge and washed with DI water (5 mL) and was eluted from the column with KHCO₃ (400 μL) and acidified to pH 4.0 with acetic acid. An AlCl₃ stock solution (2 mM, in pH 4, in 0.1 M sodium acetate buffer) was prepared and 300 μL were mixed with the ¹⁸F solution. A solution of Compound 24 (2 mM, 300 μL) in 0.1 M NaOAc was added and heated at 115° C. for 15 min and analyzed by radio-HPLC (Ultra AQ, C₁₈, 5 μm, 250×4.6 mm) using a gradient of 5% mobile phase A (50 mM ammonium acetate in water) to 95% mobile phase B (10% 50 mM ammonium acetate in water and 90% acetonitrile). The radiochemical purity of the product was 80%. After purification by HPLC, the radiochemical purity was >95%.

### Example 8: Evaluation of Fibrin-Specific Compounds in Rat Model of Carotid Endothelial Injury

Rat carotid endothelial injury model in vivo studies were conducted, wherein the common carotid artery is isolated and crushed briefly with a hemostat, resulting in mural thrombus formation at the site of the crush. The vessel wall fibrin is then imaged with our PET compounds. All six compounds were evaluated in this model.

Adult male Wistar rats were anesthetized by isoflurane (1-2% in medical air). Body temperature was maintained at 37° C. using a thermo-regulated heating pad. The right femoral vein and artery were cannulated for compound injection and blood sampling, respectively. Endothelial injury was induced by crush injury to the right common carotid artery (1-2 cm proximal to the carotid bifurcation) by clamping the vessel with a hemostat for 5 minutes. Immediately following the surgical procedures, rats were placed in a micro-PET/CT scanner. Compound (200-600 μL) was injected, PET-CT images were acquired, and blood samples were drawn from the femoral artery prior to compound injection and at 2, 5, 10, 15, 30, 60, 90, 120 min post injection. Tissues were harvested at the end for ex vivo analyses.

### Example 9: Blood Analysis to Determine Blood Clearance of Compounds in Rats

Blood samples were drawn from prior to probe injection and at 2, 5, 10, 15, 30, 60, 90, 120 min post injection. Each blood sample was weighed and counted using a gamma-counter. The activity in each sample was calculated as percent injected dose (% ID) per gram of tissue. Compounds ⁶⁸Ga-19 and ⁶⁸Ga-20 demonstrated faster blood clearance and better metabolic stability relative to the other compounds in the study (FIG. 9).

### Example 10: Functional Compound Assay Analysis for Evaluation of the Fraction of Intact Compound Circulating at Each Time Point

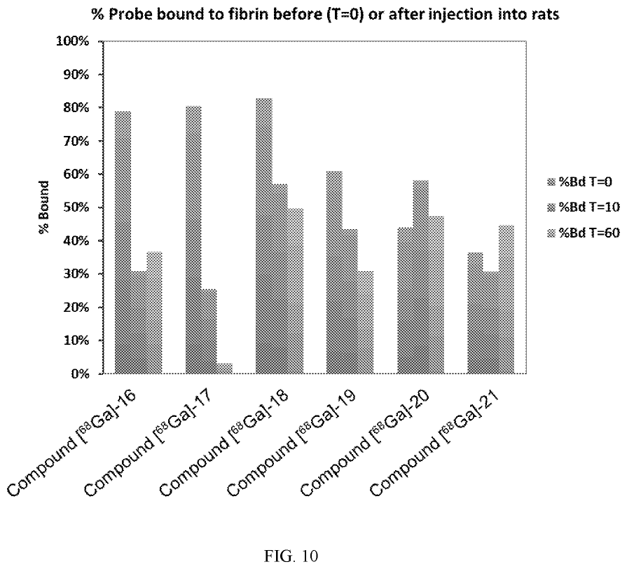

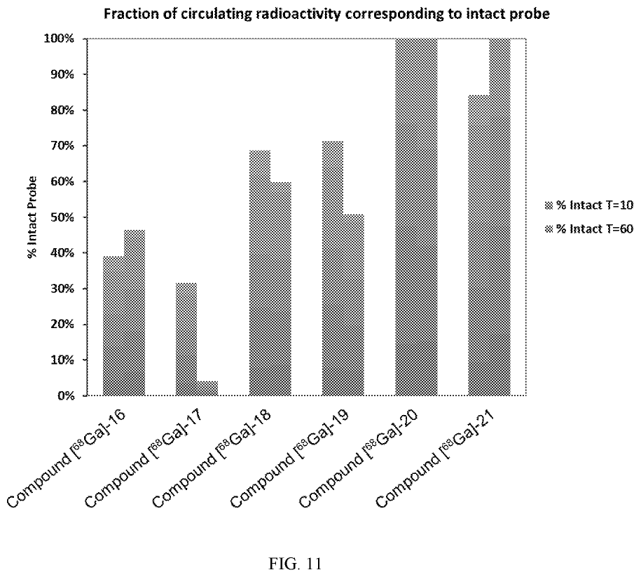

Plasma was separated (at t=10 and 60 min) and the plasma samples were incubated with fibrin immobilized in a well plate for 2 h at RT. After incubation, the counts in the supernatant in both the fibrin-containing and empty wells were measured on a gamma counter and divided by the weight of plasma to determine the concentration of unbound probe, [unbound], and total probe, [total], respectively. The amount of ⁶⁸Ga containing species bound to fibrin, [bound], was calculated from [bound]=[total]−[unbound]. As a positive control, an aliquot of the dose was spiked into blood plasma and used to estimate the total possible fibrin binding in the assay (% bound at t=0). The amount of functional probe in the blood at time t was determined by taking the ratio of the % bound to fibrin at time t compared to the % bound at t=0, and multiplying this ratio by the measured total ⁶⁸Ga % ID/g in the blood. By measuring the activity in a well plate containing fibrin and comparing it to the activity in a well plate without fibrin, the percentage of activity bound to fibrin was estimated. This % bound is compared to the % bound measured when pure compound is spiked into fresh plasma (FIG. 10 and FIG. 11).

### Example 11: Radio-HPLC Analysis of Blood for Identification of Metabolites

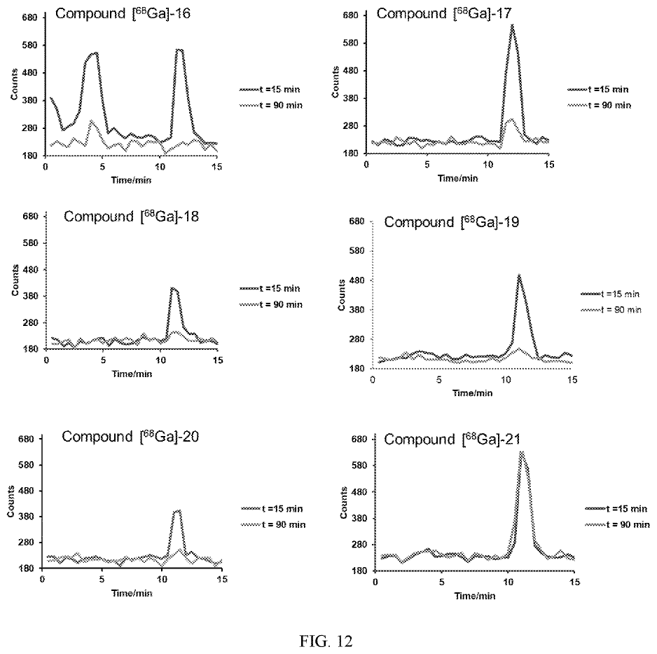

Aliquots of the 15 and 90 min plasma samples were injected into a HPLC and fractions were collected each 30 seconds. The fractions counted were compared to pure compound injected into the same column in order to identify the number of metabolites and the fraction of intact compound circulating at each time point. Unexpectedly, Compound ⁶⁸Ga-16 rapidly formed metabolites after injection while other compounds of similar structures stayed intact (FIG. 12).

### Example 12: Biodistribution

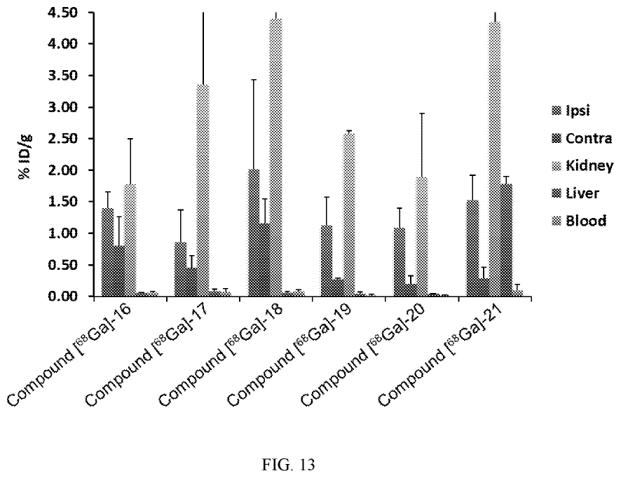

The injured carotid, the contralateral carotid, and all the organs were removed, weighed, and counted using a gamma-counter. The activity in the various organs was expressed as percent injected dose (% ID) per gram of tissue (FIG. 13).

### Example 13: Autoradiography

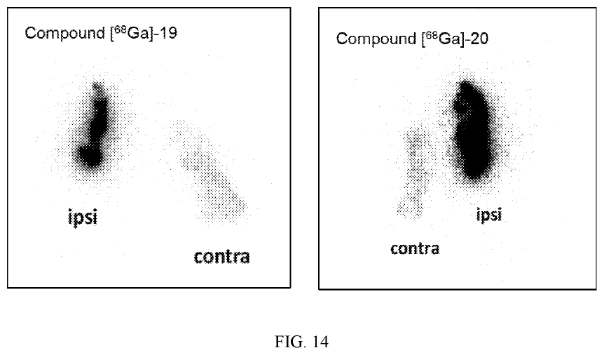

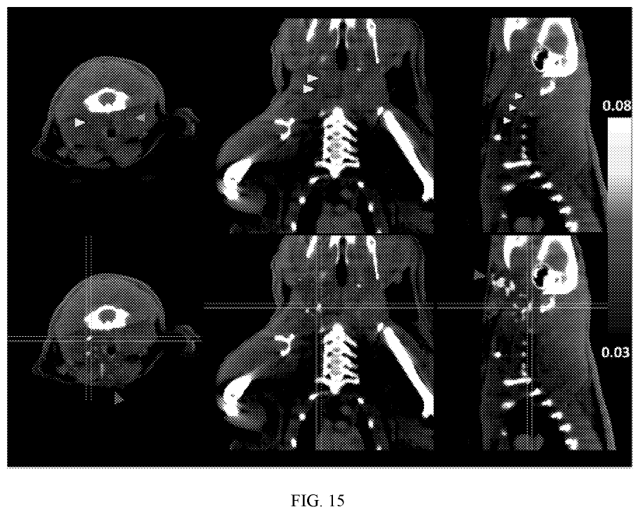

The injured ipsi- and the contra-lateral carotid arteries were placed on a multipurpose film and imaged using a Perkin-Elmer Cyclone Plus Storage Phosphor system. Additional in vivo studies were conducted using PET imaging with two Compounds ⁶⁸Ga-19 (n=6 rats) and ⁶⁸Ga-20 (n=9 rats) (FIG. 14). Of these, Compound ⁶⁸Ga-20 showed faster blood clearance, better metabolic stability and better ipsi: contra ratios.

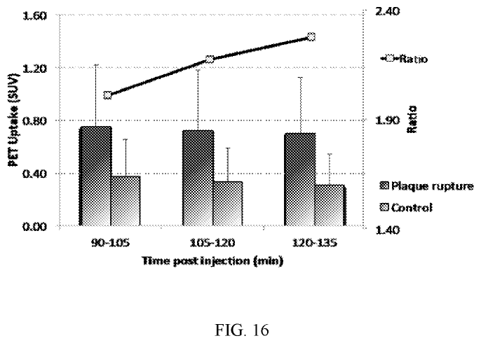

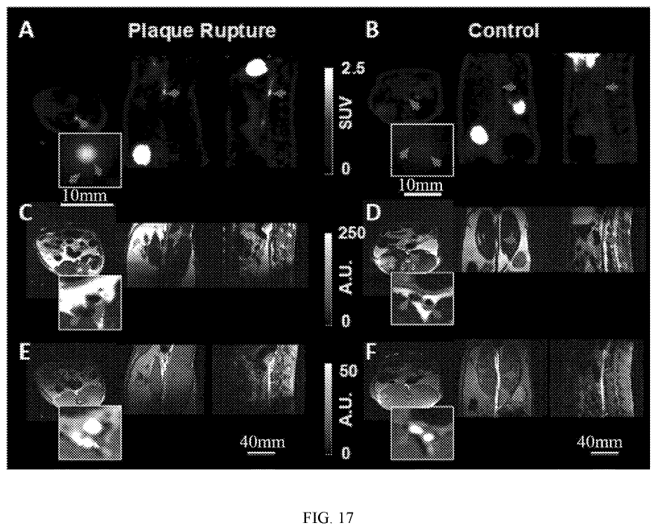

Compound ⁶⁸Ga-20 was also successfully validated in a rabbit model of high risk plaque (plaque-rupture model). Mean SUV values, TBRye and TBRm were compared across all rabbits after in vivo PET. FIG. 16 shows the average group PET uptake (SUV) across different time points as well as the ratios. As expected, higher plaque rupture-to-control SUV ratios were seen with longer post injection imaging time points. FIG. 17 shows representative PET-MR images from plaque rupture and control animals. Fibrin clots were seen as uptake spots along the aorta on the plaque rupture group, while the control aortas show a very uniform profile.

### Example 14: Ex Vivo Studies of Human Carotid Endarterectomy Specimens

Twelve discarded surgical specimens from asymptomatic patients who underwent elective carotid endarterectomy at Massachusetts General Hospital were obtained and used in ex vivo studies described herein. Samples were processed for histology, autoradiography, and probe binding assay as described above (Examples 6 and 13).

Discarded surgical specimens were embedded in optimal cutting temperature compound, snap-frozen, and stored at −80° C. before further analysis. Alternating consecutive cryosections were processed for histology and autoradiography; triplicate tissue samples with 50 μm gap in between were used for these experiments. The remaining tissue samples were processed for functional probe assay.

For histology: the specimen were fixed using 4% paraformaldehyde containing 30% sucrose. The rostral part of each segment was embedded in optimal cutting temperature compound for cryosectioning for histology (Carstairs' staining). The remaining aortic segments were cut open for gross pathology. Carstairs' staining was used to differentiate fibrin (bright red), collagen (bright blue), erythrocytes (yellow to orange), platelets (gray-blue to navy), and muscle (red). Sections were inspected using a microscope (Nikon Eclipse 50i, Kodak Scientific Imaging System), and digital images were acquired using a camera (SPOT 7.4 Slider RTKE, Diagnostic Instruments) connected to a computer.

For autoradiography, three 30 μm-thick sections were incubated with ⁶⁸Ga-20 or non-fibrin binding control probe ⁶⁸Ga-22 for 45 minutes on a lab shaker at room temperature. Autoradiography was performed after three washes of PBS. The exposure time was 2 minutes.

For functional probe assay, samples were thawed at room temperature, weighed (8-15 mg per sample), cut into duplicates, and placed into tubes containing 111-148 kBq (3-4 μCi) ⁶⁸Ga-20 or the non-fibrin binding control probe ⁶⁸Ga-22 in 1 mL PBS. The mixture was shaken for 45 minutes at room temperature. After centrifugation, supernatant (sup-1) was removed, kept for further analysis, and tissue samples were washed with PBS (1 mL); this procedure was repeated twice for each sample. Wash solutions (wash-1 and wash-2) were kept for further analysis. Activities in tissue samples and solutions sol-1, wash-1, and wash-2 were measured on a gamma counter. Percent uptake was calculated as follows:

% uptake=[(activity in tissue)/total activity (tissue+sup-1+wash-1+wash-2)]×100

### Results:

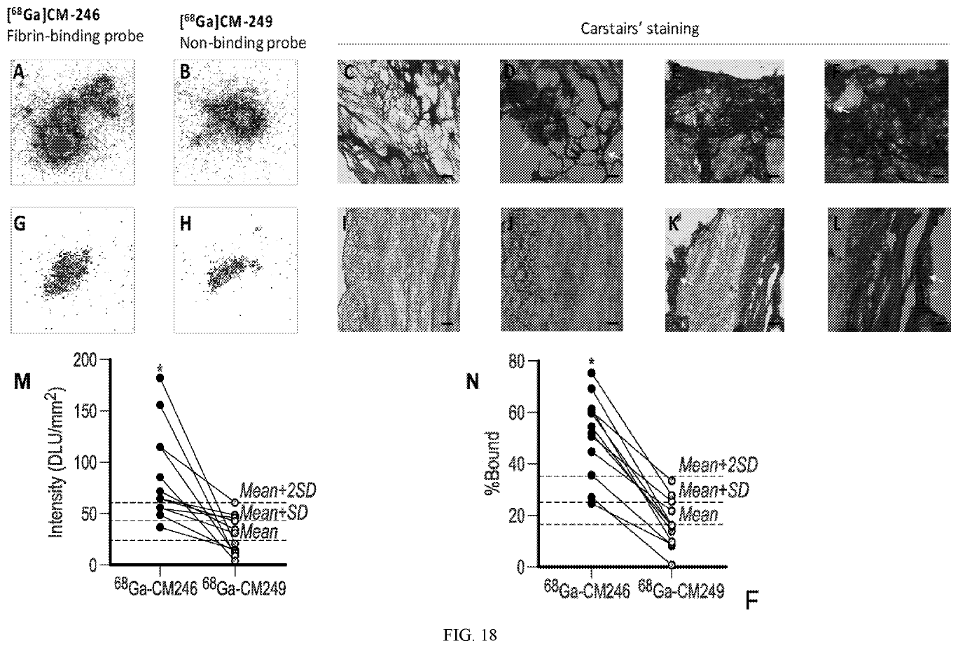

Both autoradiography (FIGS. 18A-18B, 18G-18H, and 18M) and functional probe assay (FIG. 18N) showed significantly higher uptake of ⁶⁸Ga-20 compared to non-binding probe ⁶⁸Ga-22 (P<0.05 for both). However, there was a substantial interpatient variability in ⁶⁸Ga-20 uptake in both experiments (FIGS. 18M-18N). Carstairs' staining results were congruent with autoradiography and probe assay. In some patient specimens, high ⁶⁸Ga-uptake and substantial fibrin presence were detected (FIGS. 18A-18F), whereas low-uptake specimens had comparatively less fibrin (FIGS. 18G-18L). On the other hand, the results obtained from most specimens were not this clear cut, and the overall amount of fibrin in the tissue did not correlate with autoradiography (P=0.45) or binding assay (P=0.28).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A compound of Formula: 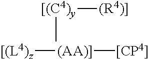
or a pharmaceutically acceptable salt thereof,
wherein
y is 1 and z is 0;
R⁴ is copper-64;
C⁴ is: 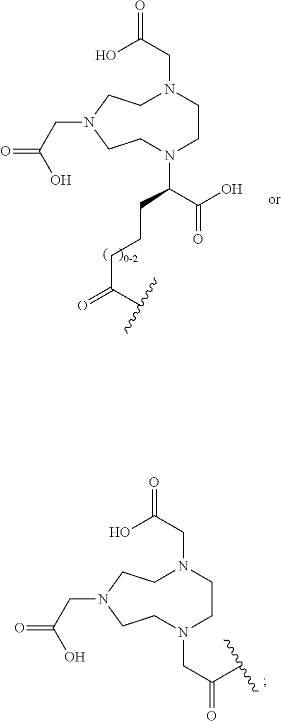
AA-CP⁴ is a polypeptide having a sequence —Y-e-C-Hyp-Y(3-Cl)-G-L-C—H—I-q- (SEQ ID NO: 5);
wherein C⁴ is bound to AA-CP⁴ at position 1 of SEQ ID NO: 5 and the C termini of the polypeptide is amidated.

2. The compound of claim 1, wherein C⁴ is

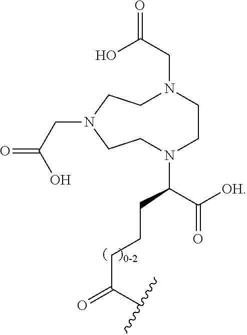

3. The compound of claim 1, wherein C⁴ is

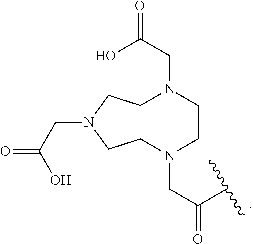 [SMILES: *C(=O)CN1CCN(CC(=O)O)CCN(CC(=O)O)CC1]

4. A compound of structure: 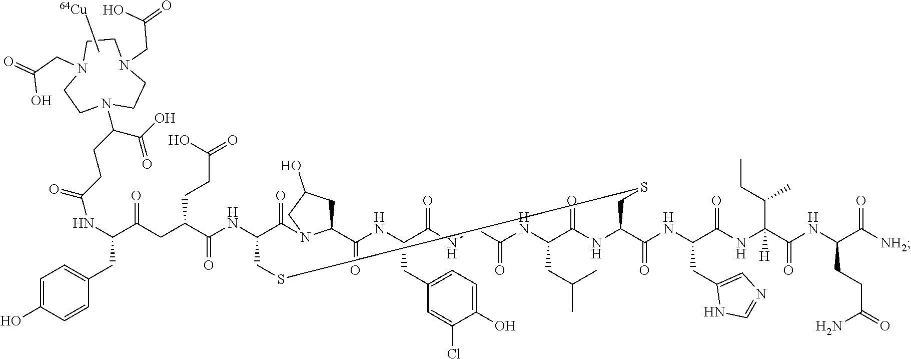
or a pharmaceutically acceptable salt thereof.

5. A pharmaceutical composition comprising a compound of claim 1, or a pharmaceutically acceptable salt thereof, and a pharmaceutically acceptable excipient.

6. The pharmaceutical composition of claim 5, wherein C⁴ is

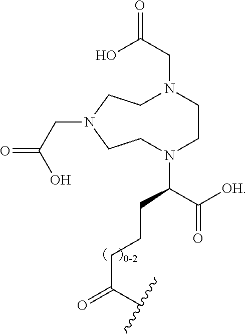

7. The pharmaceutical composition of claim 5, wherein C⁴ is

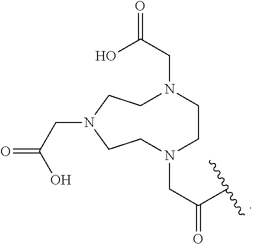 [SMILES: *C(=O)CN1CCN(CC(=O)O)CCN(CC(=O)O)CC1]

8. The pharmaceutical composition of claim 5, wherein the compound is of structure: 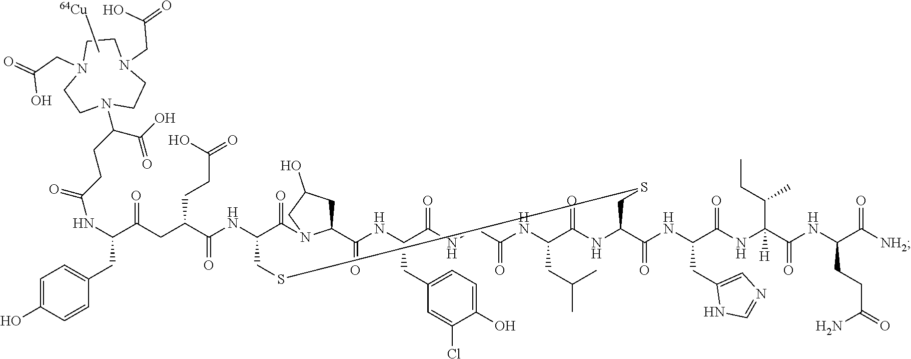
or a pharmaceutically acceptable salt thereof.
